(19) United States
(12) Patent Application Publication
(10) Pub. No.: US 2006/0135508 A1
(43) Pub. Date: Jun. 22, 2006

(54) Bicyclo-pyrazoles active as kinase inhibitors, process for their preparation and pharmaceutical compositions comprising them

(76) Inventors: Manuela Villa, Lurago d'Erba (IT); Francesca Abrate, Milan (IT); Daniele Fancelli, Milan (IT); Mario Varasi, Milan (IT); Anna Vulpetti, Milan (IT)

Correspondence Address: Peter I Bernstein;Scully Scott Murphy & Presser, 400 Garden City Plaza, Suite 300, Garden City, NY, 11530, US

(21) Appl. No.: 10/522,253

(22) PCT Filed: Jul. 16, 2003
(86) PCT No.: PCT/EP03/07851

§ 371 Date: Jan. 30, 2006

Related U.S. Application Data
(60) Provisional application No. 60/398,120, filed on Jul. 25, 2002.

(51) Int. Cl. A61K 31/55; A61K 31/4745; C07D 487/02; C07D 471/02

(52) U.S. Cl. 514/215; 546/117; 540/577; 514/303

(57) ABSTRACT

The present invention provides a method for treating diseases caused by and/or associated with an altered protein kinase activity which comprises administering to a mammal in need thereof an effective amount of a pyrrolo-pyrazole or pyrazolo-azepine. The invention also provides specific pyrrolo-pyrazoles and pyrazolo-azepines, useful intermediates, a library comprising at least two of them, a process for their preparation and the pharmaceutical compositions containing them, which are useful in the treatment of diseases caused by and/or associated with an altered protein kinase activity such as cancer, cell proliferative disorders, viral infections, autoimmune diseases and neurodegenerative disorders.

### BACKGROUND OF THE INVENTION

1. Field of the Invention

The present invention relates to bicyclo-pyrazole derivatives active as kinase inhibitors and, more in particular, it relates to pyrrolo-pyrazole and pyrazolo-azepine derivatives, to a process for their preparation, to pharmaceutical compositions comprising them and to their use as therapeutic agents, particularly in the treatment of diseases linked to deregulated protein kinases.

2. Discussion of the Background

The malfunctioning of protein kinases (PKs) is the hallmark of numerous diseases.

A large share of the oncogenes and proto-oncogenes involved in human cancers code for PKs. The enhanced activities of PKs are also implicated in many non-malignant diseases such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

PKs are also implicated in inflammatory conditions and in the multiplication of viruses and parasites. PKs may also play a major role in the pathogenesis and development of neurodegenerative disorders.

For a general reference to PKs malfunctioning or deregulation see, for instance, Current Opinion in Chemical Biology 1999, 3, 459465.

Some pyrrolo-pyrazole or pyrazolo-azepine derivative are known in the art. Few pyrazolo-azepine derivatives were studied (CAS 55:27362i, Yamamoto, H. et al, Bull. Chem. Soc. Jap., 44(1), 153-8, 1971 and Moriya, T. et al; Bull. Chem. Soc. Jap., 41(1), 230-1, 1968). Some pyrrolo-pyrazole derivatives were disclosed in Elguero, J. et al; Bull. Soc. Chim. Fr.(4), 1497-9 1971 and the antibacterial activity of some other pyrrolo-pyrazole derivatives was shown in WO01/042242 and JP06073056.

### SUMMARY OF THE INVENTION

The present inventors have now discovered that some pyrrolo-pyrazoles and pyrazolo-azepines are endowed with multiple protein kinase inhibiting activity and are thus useful in therapy in the treatment of diseases caused by and/or associated with deregulated protein kinases.

As such, it is an object of the invention to provide compounds, which are useful as therapeutic agents against a host of diseases caused by a deregulated protein kinase activity.

It is another object to provide compounds endowed with multiple protein kinase inhibiting activity.

More specifically, the pyrrolo-pyrazoles and pyrazolo-azepines of this invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratocanthoma, thyroid follicular cancer and Kaposi's sarcoma.

Due to the key role of PKs in the regulation of cellular proliferation, these pyrrolo-pyrazoles and pyrazolo-azepines are also useful in the treatment of a variety of cell proliferative disorders such as, for instance, benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

The compounds of the invention can be useful in the treatment of Alzheimer's disease, as suggested by the fact that cdk5 is involved in the phosphorylation of tau protein (J. Biochem., 117, 741-749, 1995).

The compounds of this invention, as modulators of apoptosis, may also be useful in the treatment of cancer, viral infections, prevention of AIDS development in HIV-infected individuals, autoimmune diseases and neurodegenerative disorders.

The compounds of this invention may be useful in inhibiting tumor angiogenesis and metastasis.

The compounds of the invention are useful as cyclin dependent kinase (cdk) inhibitors and also as inhibitors of other protein kinases such as, for instance, protein kinase C in different isoforms, Met, PAK-4, PAK-5, ZC-1, STLK-2, DDR-2, Aurora 1, Aurora 2, Bub-1, PLK, Chk1, Chk2, HER2, raf1, MEK1; MAPK, EGF-R, PDGF-R, FGF-R, IGF-R, VEGF-R, P13K, weel kinase, Src, Abl, Akt, ILK, MK-2, IKK-2, Cdc7, Nek, and thus be effective in the treatment of diseases associated with other protein kinases.

Accordingly, the present invention provides a method for treating diseases caused by and/or associated with an altered protein kinase activity which comprises administering to a mammal in need thereof an effective amount of a pyrrolo-pyrazole or pyrazolo-azepine derivative represented by formula (I): 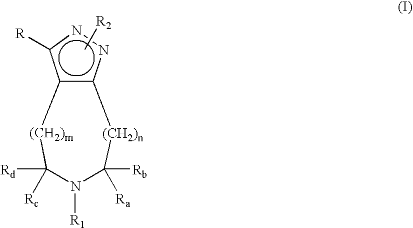
wherein R represents hydrogen or halogen atom, or an optionally substituted group selected from aryl C₂-C₆ alkenyl, (heterocyclyl) C₂-C₆ alkenyl, aryl C₂-C₆ alkynyl, or (heterocyclyl) C₂-C₆ alkynyl group, —R′, —COR′, —COOR′, —CN, —CONR′R″, —OR′, —S(O)_{q}R′, —SO₂NR′R″, —B(OR′″)₂, —SnR″″, wherein R′ and R″, the same or different, independently represent hydrogen atom or an optionally further substituted straight or branched C₁-C₆ alkyl, C₂-C₆ alkenyl, C₂-C₆ alkynyl, saturated or unsaturated C₃-C₆ cycloalkyl, aryl, heterocyclyl, aryl C₁-C₆ alkyl or (heterocyclyl)C₁-C₆ alkyl; R′″ represents hydrogen,

C₁-C₆ alkyl, or R′″, together with the two oxygen and the boron atoms, forms a saturated or unsaturated C₅-C₈ (hetero)cycloalkyl, optionally benzocondensed or substituted, and R″″ represents C₁-C₆ alkyl;

R₁ represents hydrogen atom or an optionally substituted group selected from —R′, —CH₂R′, —COR′, —COOR′, —CONR′R″, —C(═NH)NHR′, —S(O)_{q}R′, or —SO₂NR′R″, wherein R′ and R″ are as defined above;

R₂ represents hydrogen atom, —COR′, —COOR′, —CONR′R″, —S(O)_{q}R′, —SO₂NR′R″, C₁-C₆ alkyl or (heterocyclyl)C₁-C₆ alkyl group, wherein R′ and R″ are as defined above; Rₐ, R_{b}, R_{c} and R_{d}, being the same or different, independently represent hydrogen atom, an optionally further substituted straight or branched C₁-₆ alkyl, aryl, heterocyclyl, aryl C₁-C₆ alkyl, (heterocyclyl)C₁-C₆ alkyl or —CH₂OR′ group, wherein R′ is as above defined, or Rₐ and R_{b} and/or R_{c} and R_{d}, taken together with the carbon atom to which they are bonded, form an optionally substituted, saturated or unsaturated, C₃-C₆ cycloalkyl group; q is 0, 1 or 2; m and n, each independently, represents 0, 1 or 2, provided that m+n is 0 or equal to 2; or a pharmaceutically acceptable salt thereof.

In a preferred embodiment of the method described above, the disease caused by and/or associated with an altered protein kinase activity is selected from the group consisting of cancer, cell proliferative disorders, Alzheimer's disease, viral infections, auto-immune diseases and neurodegenerative disorders.

Specific types of cancer that may be treated according to the invention include carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderoma pigmentosum, keratoxanthoma, thyroid follicular cancer and Kaposi's sarcoma.

In another preferred embodiment of the method described above, the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis glomerulonephritis and post-surgical stenosis and restenosis. In addition, the method object of the present invention, provides tumor angiogenesis and metastasis inhibition. The present invention also provides a pyrrolo-pyrazole or pyrazolo-azepine derivative represented by formula (I):

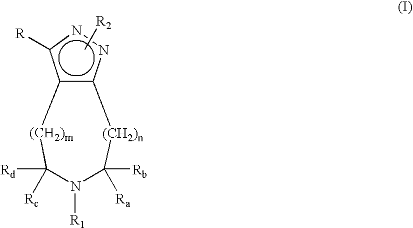

wherein R represents hydrogen or halogen atom, or an optionally substituted group selected from aryl C₂-C₆ alkenyl, (heterocyclyl) C₂-C₆ alkenyl, aryl C₂-C₆ alkynyl, or (heterocyclyl) C₂-C₆ alkynyl group, —R′, —COR′, —COOR′, —CN, —CONR′R″, —OR′, —S(O)_{q}R′, —SO₂NR′R″, —B(OR′″)₂, —SnR″″, wherein R′ and R″, the same or different, independently represent hydrogen atom or an optionally further substituted straight or branched C₁-C₆ alkyl, C₂-C₆ alkenyl, C₂-C₆ alkynyl, saturated or unsaturated C₃-C₆ cycloalkyl, aryl, heterocyclyl, aryl C₁-C₆ alkyl or (heterocyclyl)C₁-C₆ alkyl; R′″ represents hydrogen, C₁-C₆ alkyl, or R′″, together with the two oxygen and the boron atoms, forms a saturated or unsaturated C₅-C₈ (hetero)cycloalkyl, optionally benzocondensed or substituted, and R″″ represents C₁-C₆ alkyl;

R₁ represents hydrogen atom or an optionally substituted group selected from —R′, —CH₂R′, —COR′, —COOR′, —CONR′R″, C(═NH)NHR′, —S(O)_{q}R′, or —SO₂NR′R″, wherein R′ and R″ are as defined above;

R₂ represents hydrogen atom, —COR′, —COOR′, —CONR′R″, —S(O)_{q}R′, —SO₂NR′R″, C₁-C₆ alkyl or (heterocyclyl)C₁-C₆ alkyl group, wherein R′ and R″ are as defined above;

Rₐ, R_{b}, R_{c} and R_{d}, being the same or different, independently represent hydrogen atom, an optionally further substituted straight or branched C₁-C₆ alkyl, aryl, heterocyclyl, aryl C₁-C₆ alkyl, (heterocyclyl)C₁-C₆ alkyl or —CH₂OR′ group, wherein R′ is as above defined, or Rₐ and R_{b} and/or R_{c} and R_{d}, taken together with the carbon atom to which they are bonded, form an optionally substituted, saturated or unsaturated, C₃-C₆ cycloalkyl group; q is 0, 1 or 2; m and n, each independently, represents 0, 1 or 2, provided that m+n is 0 or equal to 2; with the following further provisos:
when m and n are both 1, R is hydrogen atom or hydroxy group and Rₐ, R_{b}, R_{c} and R_{d} are all hydrogen atoms, then R₁ is not hydrogen atom, acetyl, benzyl or ethoxycarbonyl group;
when m is 2 and n is 0, R, Rₐ, R_{b}, R_{c} and R_{d} are all hydrogen atoms, then R₁ is not hydrogen atom or ethoxycarbonyl group;
when m and n are both 0, R, Rₐ, R_{b}, R_{c} and R_{d} are all hydrogen atoms, then R₁ is not hydrogen atom, phenyl-oxazolidinone, quinoline, pyridobenzoxazine or naphthyridine group;

when m and n are both 0, R is propyl, Rₐ, R_{b}, R_{c} and R_{d} are all hydrogen atoms, then R₁ is not phenyl-oxazolidinone group and
when m and n are both 0, R is hydroxy, methyl or ethyl group and Rₐ, R_{b}, R_{c} and R_{d} are all hydrogen atoms, then R₁ is not a methoxycarbonyl group;

or a pharmaceutically acceptable salt thereof.

The pyrrolo-pyrazole and pyrazolo-azepine derivatives of formula (I), object of the invention, are obtainable through a synthetic process comprising well known reactions carried out according to conventional techniques, as well as through an extremely versatile solid-phase and/or combinatorial process, being all comprised within the scope of the invention.

The present invention also provides a pharmaceutical composition comprising the pyrrolo-pyrazole or pyrazolo-azepine derivatives of formula (I) and at least one pharmaceutically acceptable excipient, carrier or diluent.

A more complete appreciation of the invention and many of the attendant advantages thereof will be readily obtained as the same becomes better understood by reference to the following detailed description.

### DETAILED DESCRIPTION OF THE INVENTION

The compounds of formula (I), object of the present invention, may have asymmetric carbon atoms and may therefore exist either as racemic admixtures or as individual optical isomers. Accordingly, all the possible isomers and their admixtures and of both the metabolites and the pharmaceutically acceptable bio-precursors (otherwise referred to as pro-drugs) of the compounds of formula (I), as well as any therapeutic method of treatment comprising them, are also within the scope of the present invention.

As it will be readily appreciated, depending on the values of m and n, the ring condensed to the pyrazole may consist of 5 or 7 atoms; as to the pyrazole ring, two isomers are possible and therefore the R₂ substituent may be on one of the two nitrogens. Accordingly, in the present invention and unless otherwise indicated, the general formula I comprises the compounds of formula IA, IB, IC, ID, IE and IF:

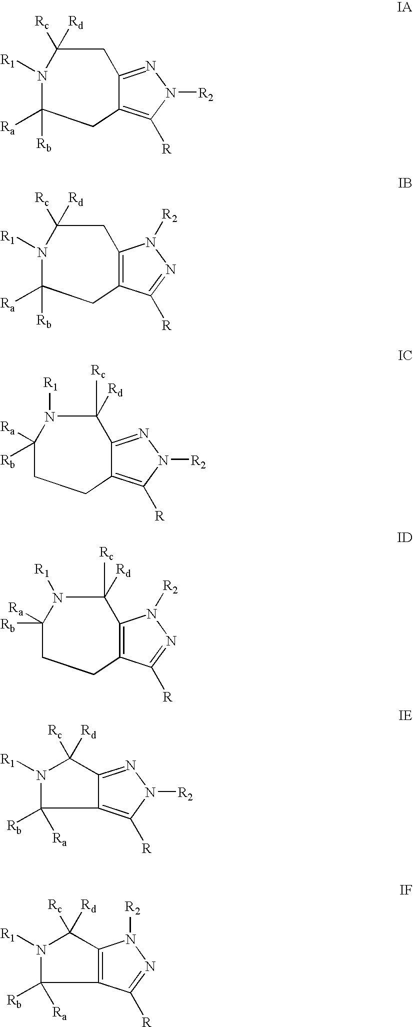

wherein R, R₁, R₂, Rₐ, R_{b}, R_{c} and R_{d} are as defined above.

As used herein, unless otherwise specified, with the term straight or branched C₁-C₆ alkyl , we intend a group such as, for instance, methyl, ethyl, n-propyl, isopropyl, n-butyl, isobutyl, sec-butyl, tert-butyl, n-pentyl, neopentyl, n-hexyl, isohexyl, and the like.

With the term aryl we intend an aromatic carbocycle such as, for instance, phenyl, biphenyl, 1-naphthyl, 2-naphthyl, and the like. Clearly, aryl groups may also refer to aromatic carbocyclic further fused or linked to non aromatic heterocyclic rings, typically 5 to 7 membered heterocycles.

With the term heterocyclyl, hence encompassing aromatic heterocycles, we further intend a saturated or partially unsaturated 5 to 7 membered carbocycle wherein one or more carbon atoms are replaced by heteroatoms such as nitrogen, oxygen and sulphur, for instance, 1,3-dioxolane, pyran, thiophene, furan, pyrrole, imidazole, pyrazole, thiazole, isothiazole, oxazole, isoxazole, pyridine, pyrazine, pyrimidine, pyridazine, pyrrolidine, pyrroline, imidazolidine, imidazoline, piperidine, piperazine, morpholine, tetrahydrofurane, tetrahydropyran, tetrahydrothiopyran, imidazolidine, pyrazolidine, pyrazoline, piperidine, azabicyclononane and the like.

Also the heterocycles may be optionally fused and, unless otherwise indicated, we intend any of the above defined heterocycles further condensed, through any one of the available bonds, with 5- or 6-membered, saturated or unsaturated heterocyclyl ring, or to a C₃-C₆ cycloalkyl ring, or to a benzene or naphthalene ring such as, for instance, quinoline, isoquinoline, chroman, chromene, thionaphthalene, indoline, and the like.

With the term C₂-C₆ alkenyl, we intend a straight or branched alkenyl group such as vinyl, allyl, crotyl, 2-methyl-1-propenyl, 1-methyl-1-propenyl, butenyl, pentenyl. The C₂-C₆ alkynyl group is a straight or branched alkynyl group such as ethynyl, propargyl, 1-propynyl, 1-butynyl, 2-butynyl.

With the term saturated or unsaturated C₃-C₆ cycloalkyl group we intend, for instance, cyclopropyl, cyclobutyl, cyclopentyl, cyclohexyl, cyclopentenyl cyclohexenyl, and the like. Unless otherwise specified, saturated or unsaturated cycloalkyl groups can be further condensed with 1 or 2 benzene rings are, for instance, 1,2,3,4tetrahydro-naphthalene-2-yl, fluorene-9-yl, and the like.

The term “C₅-C₈ (hetero)cycloalkyl” as used herein refers to a 5- to 8-membered, substituted or unsubstituted, saturated or unsaturated heterocyclyl ring, containing at least one boro and two oxygen atoms, any ring carbon may be oxidized as a carbonyl, and wherein said ring may be optionally fused to a second 5- or 6-membered, saturated or unsaturated heterocyclyl ring, or to a C₃-C₇ cycloalkyl ring, or to a benzene or naphthalene ring.

The term “aryl C₁-C₆ alkyl” refer to a straight or branched chain alkyl moiety having from 1 to 6 carbon atoms substituted with at least one aryl group as defined above, such as, for instance, benzyl, phenylethyl, benzhydryl, benzyloxy and the like. The “aryl C₂-C₆ alkenyl group” is an alkenyl group of 2 to 6 carbon atoms linked to a monocyclic or bicyclic aromatic hydrocarbon group of 6 to 10 carbon atoms. Examples of aryl alkenyl groups are styryl, 2-phenyl-1-propenyl, 3-phenyl-2-butenyl, 2-naphthylethenyl.

The “aryl C₂-C₆ alkynyl group” is an alkynyl group of 2 to 6 carbon atoms linked to a monocyclic or bicyclic aromatic hydrocarbon group of 6 to 10 carbon atoms. Examples of aryl alkynyl groups are 2-phenylethynyl, 2-naphthylethynyl.

The (heterocyclyl) C₁-C₆ alkyl group is an alkyl group of 1 to 6 carbon atoms linked to a heterocyclyl group. The (heterocyclyl) C₂-C₆ alkenyl group is an alkenyl group of 2 to 6 carbon atoms linked to a heterocyclic group. The (heterocyclyl) C₂-C₆ alkynyl group is an alkynyl group of 2 to 6 carbon atoms linked to a heterocyclic group.

From all of the above, it is clear to the skilled man that any of the groups or substituents being defined, for instance, as arylalkyl, alkoxy, cycloalkoxy, aryloxy, arylalkyloxy and the like, have to be construed from the names of the groups from which they originate.

As an example, unless specifically noted otherwise, any arylalkyloxy group has to be intended as an alkyloxy wherein the alkyl moiety is substituted by at least one aryl, both aryl and alkyl being as above defined.

With the term halogen atom, we intend fluoro, bromo, chloro or iodo atom.

The term “optionally substituted” means that the group may be substituted or unsubstituted; the substituents which may be present in the alkyl, cycloalkyl, aryl, arylalkyl, arylalkenyl, arylalkyl, alkoxy, aryloxy, cycloalkoxy, alkenyl, alkynyl or heterocyclyl groups in any of the above definitions include the following:

halo (i.e., fluoro, bromo, chloro or iodo);

hydroxy,

oxo (i.e.,═O);

nitro;

azido;

mercapto (i.e., —SH), and acetyl or phenylacetyl esters thereof (i.e., —SCOCH₃ and —SCOCH₂C₆H₅);

amino (i.e., —NH2 or —NHR^{I} or —NR^{I}R^{II}, wherein R^{I} and R^{II}, which are the same or different, are straight or branched C₁-C₆ alkyl, phenyl, biphenyl (i.e., —C₆H₄—G₆H₅), or benzyl groups, optionally substituted by hydroxy, methoxy, methyl, amino, methylamino, dimethylamino, chloro or fluoro; or R^{I} and R^{II} taken together with the nitrogen atom to which they are attached form a heterocyclic ring such as morpholino, pyrrolidino, piperidino, pyperazino or N-methylpyperazino;

guanidino, i.e., —NHC(═NH)NH₂;

formyl (i.e. —CHO);

cyano;

carboxy (i.e. —COOH), or esters thereof (i.e., —COOR^{I}), or amides thereof (i.e., —CONH₂, —CONHR^{I} or —CONHR^{I}R^{II}), wherein R^{I} and R^{II} are as defined above, and including morpholino-amides, pyrrolidino-amides, and carboxymethylamides —CONHCH₂COOH;

sulfo (i.e., —SO₃H);

acyl, i.e., —C(O)R^{I}, wherein R^{I} is as defined above, including monofluoroacetyl, difluoroacetyl, trifluoroacetyl;

carbamoyloxy (i.e., —OCONH₂) and N-methylcarbamoyloxy,

acyloxy, i.e., —OC(O)R^{I} wherein R^{I} is as defined above, or formyloxy,

acylamino, i.e., —NHC(O)R^{I}, or —NHC(O)OR^{I}, wherein R^{I} is as defined above or is a group —(CH₂)ₜCOOH where t is 1, 2 or3;

ureido, i.e., —NH(CO)NH₂, —NH(CO)NHR^{I}, —NH(CO)NR^{I}R^{II}, wherein R^{I} and R^{II} are as defined above, including —NH(CO)-(4morpholino), —NH(CO)—(1-pyrrolidino), —NH(CO)—(1-piperazino), —NH(CO)-(4-methyl-1-piperazino);

sulfonamido, i.e., —NHSO₂R^{I} wherein R^{I} is as defined above;

a group —(CH₂)ₜCOOH, and esters and amides thereof, i.e., —(CH₂)ₜCOOR^{I} and —(CH₂)ₜCONH₂, —(CH₂)ₜCONHR^{I}, —CH₂)ₜCONR^{I} R^{II}, wherein t, R^{I} and R^{II} are as defined above;

a group —NH(SO₂)NH₂, —NH(SO₂)NHR^{I}, —NH(SO₂)NR^{I}R^{II}, wherein R^{I} and R^{II} are as defined above, including —NH(SO₂)-(4morpholino), —NH(SO₂)(1-pyrrolidino), —NH(SO₂)(1-piperazino), —NH(SO₂)-(4methyl-1-piperazino);

a group —OC(O)OR^{I}, wherein R^{I} is as defined above;

a group —OR^{I}, wherein R^{I} is as defined above, including —OCH₂COOH;

a group —O—CH₂—O—, methylendioxy or —O—CH₂— CH₂—O—, ethylendioxy,

a group —SR^{I}, wherein R^{I} is as defined above, including —SCH₂COOH;

a group —S(O)R^{I}, wherein R^{I} is as defined above;

a group —S(O₂)R^{I}, wherein R^{I} is as defined above;

a group —SO₂NH₂, —SO₂NHR^{I}, or —SO₂NR^{I}R^{II}, wherein R^{I} and R^{II} are as defined above;

C₁-C₆ alkyl or C₂-C₆ alkenyl;

C₃-C₇ cycloalkyl;

substituted methyl selected from chloromethyl, fluoromethyl, difluoromethyl, trifluoromethyl, aminomethyl, N,N-dimethylaminoethyl, azidomethyl, cyanomethyl, carboxymethyl, sulfomethyl, carbamoylmethyl, carbamoyloxymethyl, hydroxymethyl, methoxycarbonylmethyl, ethoxycarbonylmethyl, tert-butoxycarbonylmethyl and guanidinomethyl.

When present, carboxy, hydroxy, mercapto and amino groups may be either free or in a protected form. Protected forms of said groups are any of those generally known in the art.

Preferably, carboxy groups are protected as esters thereof, in particular methyl, ethyl, tert-butyl, benzyl, and 4nitrobenzyl esters. Preferably, hydroxy groups are protected as silyl-ethers, ethers or esters thereof, in particular trimethyl silyl, tert-butyldiphenyl silyl, triethyl silyl, triisopropyl silyl or tert-butyldimethylsilyl ethers, methoxymethyl ethers, tetrahydropyranyl ethers, benzyl ethers, acetates or benzoates. Preferably, mercapto groups are protected as thioethers or thioesters, in particular tert-butyl thioethers, thioacetates or thiobenzoates. Preferably, amino groups are protected as carbamates, e.g. tert-butoxycarbonyl derivatives, or as amides, e.g. acetamides and benzamides.

Furthermore, hydrates, solvates of compounds of formula (I), and physiologically hydrolyzable derivatives (i.e., prodrugs) of compounds of formula (I) are included within the scope of the present invention.

Pharmaceutically acceptable salts of the compounds of formula (I) are the acid addition salts with inorganic or organic, e.g. nitric, hydrochloric, hydrobromic, sulphuric, perchloric, phosphoric, acetic, trifluoroacetic, propionic, glycolic, lactic, oxalic, malonic, malic, maleic, tartaric, citric, benzoic, cinnamic, mandelic, methanesulphonic, isethionic and salicylic acid, as well as the salts with inorganic or organic bases, e.g. alkali or alkaline-earth metals, especially sodium, potassium, calcium or magnesium hydroxides, carbonates or bicarbonates, acyclic or cyclic amines, preferably methylamine, ethylamine, diethylamine, triethylamine or piperidine.

Preferred compounds of formula (I) are the compounds wherein R is H, I, Br, Cl, F, aryl, C₂-C₆ alkenyl, C₂-C₆ alkynyl, —B(OR′″)₂, —COR′, —CONR′R″, —CN, SO₂R′, OR′, SR′, and R₁ is H, C₁-C₆ alkyl, aryl, —COR′, —CONR′R″, —COOR′, —SO₂R′, or —SO₂NR′R″, and R₂ is H, —COOR′, —COR′, —CONR′R″, C₁-C₆ alkyl, —SO₂R′, or —SO₂NR′R″, (heterocyclyl) C₁-C₆ alkyl group , wherein R′ and R″, the same or different, are selected from hydrogen or optionally substituted straight or branched C₁-C₆ alkyl, aryl or aryl C₁-C₆ alkyl groups;

Rₐ, R_{b}, R_{c} and R_{d}, the same or different, are selected from hydrogen or straight or branched C₁-C₃ alkyl or, taken together with the carbon atom to which they are bonded form a C₃-C₆ cycloalkyl group.

Other preferred compounds of formula (I) are the compounds wherein R is selected from aryl, heterocyclyl, —COR′, —CONR′R″, wherein R′ and R″, the same or different, are selected from hydrogen or optionally substituted straight or branched C₁-C₆ alkyl, aryl or aryl C₁-C₆ alkyl groups.

Other preferred compounds of formula (I) are the compounds wherein R₁ is selected from H, C₁-C₆ alkyl, aryl, —COR′, —CONR′R″, COOR′, —SO₂R′ or —SO₂NR′R″, wherein R′ and R″, the same or different, are selected from hydrogen or optionally substituted straight or branched C₁-C₆ alkyl, aryl or aryl C₁-C₆ alkyl groups.

Another preferred class of compounds of formula (I) are the compounds wherein R₂ is H, —COOR′, —CONR′R″, C₁-C₆ alkyl, wherein R′ and R″, the same or different, are selected from hydrogen or optionally substituted straight or branched C₁-C₆ alkyl, aryl or aryl C₁-C₆ alkyl groups.

As formerly indicated, it is a further object of the invention a process for preparing the compounds of formula (I) and pharmaceutically acceptable salts thereof

### General Reaction Scheme

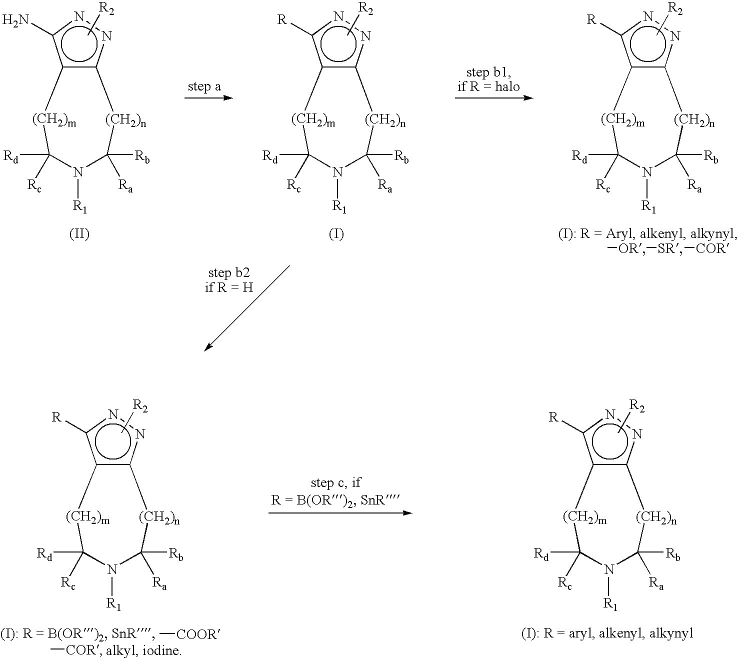

In particular, the present invention provides a process which comprises:

a) submitting a compound of formula (II)

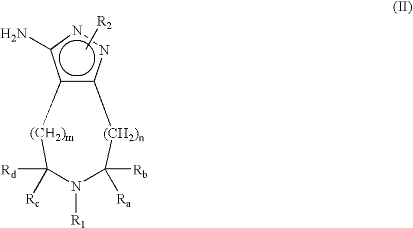

wherein R₁ is as defined above but not hydrogen, and Rₐ, R_{b}, R_{c}, R_{d}, R₂, m and n are as defined above, to diazotation and subsequent appropriate quenching, thus obtaining a compound of formula (I)

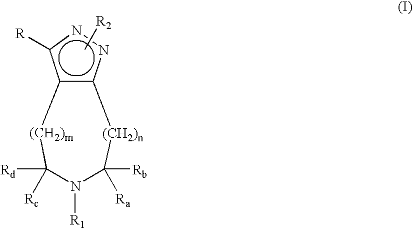

wherein R₁ is as defined above but not hydrogen; Rₐ, R_{b}, R_{c}, R_{d}, R₂, m and n are as defined above, and R is hydrogen, iodine, bromine, chlorine or fluorine atom or a CN group;

b1) converting a thus obtained compound of formula (I) wherein R is L Br, Cl into another compound of formula (I) wherein R is an optionally substituted aryl, C₂-C₆ alkenyl, C₂-C₆ alkynyl, —SR′, —OR′ or —COR′ wherein R′ is as defined above;

b2) converting a compound of formula (I) wherein R is hydrogen into another compound of formula (I) wherein R is —B(OR′″)₂, —SnR″″, —COOR′, —COR′, C₁-C₆ alkyl or iodine, wherein R′, R′″ and R″″ are as defined above;

c) converting a compound of formula (I) wherein R is —B(OR′″)₂ or —SnR″″ as above defined into another compound of formula (I) wherein R is an optionally substituted aryl C₂-C₆ alkenyl, C₂-C₆ alkynyl;

d) optionally converting a compound of formula (I) into another different compound of formula (I), and, if desired, converting a compound of formula (I) into a pharmaceutically acceptable salt thereof or converting a salt into the free compound (I).

The above process can be carried out according to well known methods. It is clear to the person skilled in the art that if a compound of formula (I), prepared according to the above process, is obtained as an admixture of isomers, their separation into the single isomers of formula (I), carried out according to conventional techniques, is still within the scope of the present invention.

Likewise, the salification of a compound of formula (I) or the conversion of its salt into the free compound (I) carried out according to well-known procedures in the art, are still within the scope of the invention.

According to a preferred aspect of the process of the invention avoiding the unwanted by-products formation, a compound of formula (I), obtained according to step a above, could be first supported onto a suitable solid support, such as resin and then, after the reactions as per steps b1, b2, c and d above described, reconverted into a compound of formula (I).

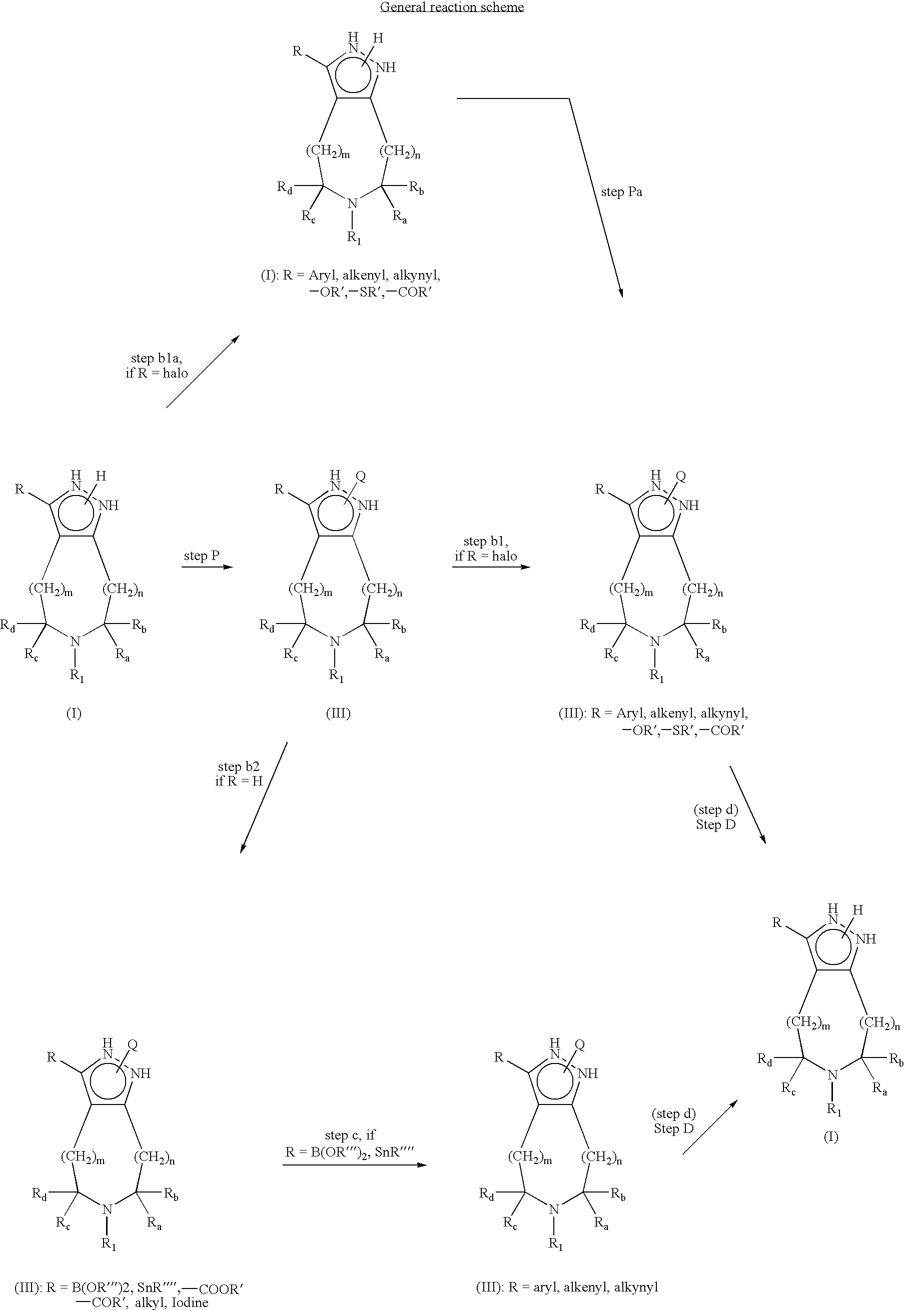

It is therefore a further object of the invention a process for preparing a compound of formula (I) as defined above, which process comprises:

either

b1a) converting a compound of formula (I) into another compound of formula (I) wherein R has the above reported meanings resulting from step b1 and R₁, Rₐ, R_{b}, R_{c}, R_{d}, m and n are as defined above analogously to step b1 above described and

Pa) reacting the resultant compound of formula (I) wherein R, Rₐ, R_{b}, R_{c}, R_{d}, m and n are as defined above, R₁ is as described above but not hydrogen and R₂ is hydrogen, with a suitable solid support so as to obtain a compound of formula (III)

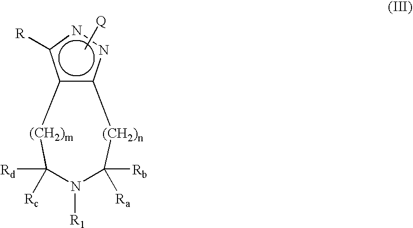

wherein R, Rₐ, R_{b}, R_{c}, R_{d}, m and n are as defined above, R₁ is as defined above but not hydrogen, and Q is a solid support, or

P) reacting a compound of formula (I) wherein R, Rₐ, R_{b}, R_{c}, R_{d}, m and n are as defined above, R₁ is as defined above but not hydrogen and R₂ is hydrogen, with a suitable solid support so as to obtain a compound of formula (III) as defined above and

B) then, analogously to steps b1, b2, c and d above described, optionally converting a thus obtained compound of formula (III) into another compound of formula (III) wherein R has the above reported meanings for steps b1, b2, c and d and R₁, Rₐ, R_{b}, R_{c}, R_{d}, m and n are as defined above;

D) cleaving the resultant compound of formula (III) so as to eliminate the solid support and to obtain the desired compound of formula (I);

E) optionally converting a compound of formula (I) into another different compound of formula (I),

and, if desired, converting a compound of formula (I) into a pharmaceutically acceptable salt thereof or converting a salt into the free compound (I) as described above.

It is a further object of the present invention to provide useful intermediates of formula III

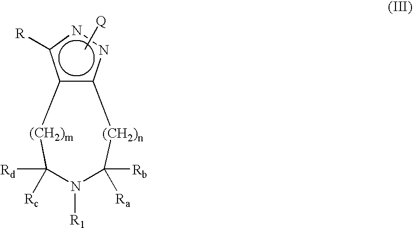

wherein R, R₁ Rₐ, R_{b}, R_{c}, R_{d}, m and n are as defined above, and Q is a solid support, more preferably a residue derived from a resin selected from the group consisting of isocyanate polystyrenic resin, 2-chloro-trityl chloride resin, trityl chloride resin, p-nitrophenyl carbonate Wang resin and the bromo-4-methoxyphenyl)methyl polystyrene. A process for the preparation of a compound of formula (III) as defined above is also provided, which process comprises:

either

b1a) converting a compound of formula (I) into another compound of formula (I) wherein R has the above reported meanings resulting from step b1 and R₁, Rₐ, R_{b}, R_{c}, R_{d}, m and n are as defined above, analogously to step b1 above described and

Pa) reacting the resultant compound of formula (I) wherein R, Rₐ, R_{b}, R_{c}, R_{d}, m and n are as defined above, R₁ is as defined above but not hydrogen and R₂ is hydrogen, with a suitable solid support so as to obtain a compound of formula (III)

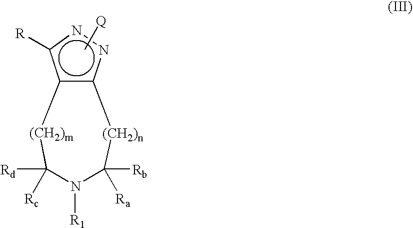

wherein R, Rₐ, R_{b}, R_{c}, R_{d}, m and n are as defined above, R₁ is as defined above but not hydrogen, and Q is a solid support, or

P) reacting a compound of formula (I) wherein R, Rₐ, R_{b}, R_{c}, R_{d}, m and n are as defined above, R₁ is as described above but not hydrogen and R₂ is hydrogen, with a suitable solid support so as to obtain a compound of formula (III) as defined above and

B) then, analogously to steps b1, b2, c and d above described, optionally converting a thus obtained compound of formula (III) into another compound of formula (III) wherein R has the above reported meanings for steps b1 to d and R₁, Rₐ, R_{b}, R_{c}, R_{d}, m and n are as defined above.

According to step a) of the process, a compound of formula (I) wherein R is hydrogen, I, Br, Cl, F, CN, and R₁ is as defined above but not hydrogen, and Rₐ, R_{b}, R_{c}, R_{d}, R₂, m and n are as defined above, may be prepared by reacting a compound of formula (II), wherein R₁ is as defined above but not hydrogen, and Rₐ, R_{b}, R_{c}, R_{d}, R₂, m and n are as defined above, with organic or inorganic nitrates such as sodium nitrate or isopentylnitrate, in the presence of a suitable hydrogen source, such as HPO₂, thiophenol, sodium stannite, Bu₃SnH, Et₃SiH, or of a suitable halogenating or cyanating agent such as tetrabutylamonium iodide and/or iodine, tetrabutylamonium bromide and/or bromine, tetrabutylamonium chloride and/or chlorine, CuBr, CuCl, CuI, CuCN, sodium tetrafluoroborate, ammonium tetrafluoroborate, in aqueos acidic solution at various concentrations such as diluted chloridic acid or diluted citric acid, or in organic solvents such as tetrahydrofurane, 1,4-dioxan, dichloromethane, chloroform, toluene, acetonitrile, ethylacetate, acetone, dimethylformamide, ethanol, methanol water at a temperature ranging from about −78° C. to reflux, for a suitable time ranging from 5 min to 72 hours. More preferably, the step a) is carried out on compounds of the formula (II) wherein R₂ is not hydrogen atom.

According to step b1) of the process, a compound of formula (I) wherein R is an optionally substituted aryl or C₂-C₆ alkenyl group, and R₁, R₂, Rₐ, R_{b}, R_{c}, R_{d}, m and n are as defined above, can be obtained by reacting a compound of formula (I), wherein R is halogen atom, and R₁, R₂, Rₐ, R_{b}, R_{c}, R_{d}, m and n are as defined above, with a suitable aryl boronic acid or ester, alkenyl boronic acid or ester, arylstannane, in the presence of a suitable catalysing agent such as palladium(0)tetrakis, bis triphenylphosphine palladium(II) dichloride, bis tricyclohexylphosphine palladium(II) dichloride, bis tri-o-tolylphosphine palladium(II) dichloride, palladium(II) acetate, tris(dibenzylideneacetone)dipalladium(0), [1,1′-bis(diphenylphosphino)ferrocene]dichloropalladium(II), [1,1′-bis(diphenylphosphino)ferrocene]dichloronickel(II), 1,4-bis(diphenylphosphino)butane palladium(II), and of a suitable base such as sodium carbonate, cesium carbonate, potassium carbonate, potassium phosphate, triethylamine, sodium hydroxide, cesium fluoride, potassium tert-butylate, sodium ethylate, potassium acetate, in a suitable solvent, such as 1,4dioxan, tetrahydrofurane, DMF (N,N-dimethylformamide), dimethoxyethane, toluene, methanol, ethanol, water, N-methylpyrrolidone, and, when needed, adding a suitable ligand, such as tributylphosphine, triphenylphosphine, tri-o-tolylphosphine, tricyclohexyl, biphenyl(dicyclohexyl)phosphine, biphenyl(ditert-butyl)phosphine, diphenylphosphine ferrocene, and/or Cu(I) salts such as CuI, Cu(I)thiophene-2-carboxylate at a temperature ranging from room temperature to reflux, for a suitable time ranging from 15 minutes to 72 hours.

According to step b1) of the process, a compound of formula (I) wherein R is an optionally substituted C₁-C₆ alkynyl, and R₁, R₂, Rₐ, R_{b}, R_{c}, R_{d}, m and n are as defined above, can be obtained by reacting a compound of formula (I), wherein R is halogen, and R₁, R₂, Rₐ, R_{b}, R_{c}, R_{d}, m and n are as defined above, with a suitable alkyne under the condition of the Sonogashira's reaction, in the presence of a suitable catalysing agent such as bistriphenylphosine palladium(II) dichloride, palladium(0) tetrakis, palladium(II) acetate, tris(dibenzylideneacetone)dipalladium(0), and of a suitable Cu(I) salt, such as CuI, and in presence of a suitable base such as sodium carbonate, potassium carbonate, cesium carbonate, potassium phosphate, triethylamine, diisopropylamine, pyridine, in a suitable solvent, such as 1,4-dioxan, tetrahydrofurane, DMF, dimethoxyethane, toluene, ethanol, methanol, and, if needed, adding a suitable ligand such as triphenylphosphine, tri-o-tolylphosphine, tricyclohexyl, diphenylphosphineferrocene, at a temperature ranging from room temperature to reflux, for a suitable time ranging from 15 minutes to 72 hours.

According to step b1) of the process, a compound of formula (I) wherein R is SR′, OR′, and R₁, R₂, Rₐ, R_{b}, R_{c}, R_{d}, R′, m and n are as defined above, can be obtained by reacting a compound of formula (I), wherein R is halogen, and R₁, R₂, Rₐ, R_{b}, R_{c}, R_{d}, m and n are as defined above, with a suitable alcohol or thiol R′OH or R′SH wherein R′ is as above defined, in the presence of a suitable base, such as potassium carbonate, sodium carbonate, cesium carbonate, potassium hydroxide, sodium hydroxide, sodium hydride, sodium methylate, sodium tert-butylate, diisopropylethylamine, pyridine, piperidine, N-methylmorpholine, dimethylaminopyridine, and, if needed, in the presence of catalysing agent, such as bis tricyclohexylphosphine palladium(II) dichloride, bis tri-o-tolylphosphine palladium(II) dichloride, palladium(II) acetate, tris(dibenzylideneacetone)dipalladium(0), [1,1′-bis(diphenylphosphino)ferrocene]dichloropalladium(II), and of a suitable ligand, such as, triphenylphosphine, tri-o-tolylphosphine, tricyclohexyl, diphenylphosphineferrocene, in a suitable solvent, such as dimethylformamide, NMP, dichloromethane, tetrahydrofurane, benzene, toluene, pyridine, dimethylsulfoxide at a temperature ranging from −20° C. to reflux, for a suitable time ranging from 15 minutes to 72 hours.

According to step b1) of the process, a compound of formula (I) wherein R is —COR′, and R₁, R₂; Rₐ, R_{b}, R_{c}, R_{d}, m and n are as defined above, can be obtained by reacting a compound of formula (I) wherein R is halogen and R₁, R₂, Rₐ, R_{b}, R_{c}, R_{d}, m and n are as defined above, with a suitable base, such as n-butyl lithium, LDA (lithium diisopropylamide), sec-butyl lithium, t-butyl lithium, lithium 2,2,6,6-tetramethylpiperidin amide, phenyl lithium, magnesium, isopropylmagnesium bromide in a suitable solvent, such as diethyl ether, tetrahydrofurane, 1,4dioxan, n-hexane, cyclohexane, pentane, toluene, DME (ethylene glycol dimethyl ether), dimethylsulfoxide in the presence of a base if needed, such as TMEDA (N,N,N′,N′-tetramethylethylenediamine), at a suitable temperature ranging from −78° C. to room temperature, for a time ranging from 15 minutes to 3 hours; the resulting lithium derivative can be quenched with a suitable electrophilic agent, such as, trialkylarylstannane/carbon monoxide, acid chlorides, acid fluorides, acid bromides, anhydrides, carbonates, halo carbonates, carbamates, DMF, and if needed, in the presence of a suitable catalysing agent, such as Pd(0)tetrakis, and of a suitable coordinating agent, such as ZnCl₂, ZnBr₂, CuCN.2LiCl, CuI CuBr, CuBr.SMe₂ at a suitable temperature ranging from about −78° C. to reflux, for a time ranging from 15 minutes to about 72 hours.

According to step b2) of the process, a compound of formula (I) wherein R is iodine, B(OR′″)₂, SnR″″, —COOR′, —COR′, C₁-C₆ alkyl and R₁, R₂, Rₐ, R_{b}, R_{c}, R_{d}, R′, R′″, R″″, m and n are as defined above, can be obtained by reacting a compound of formula (I) wherein R is hydrogen and R₁, R₂, Rₐ, R_{b}, R_{c}, R_{d}, m and n are as defined above, with a suitable lithiating agent, such as n-butyl lithium, LDA, sec-butyl lithium, t-butyl lithium, lithium 2,2,6,6-tetramethylpiperidinamide, phenyl lithium, in a suitable solvent, such as diethyl ether, tetrahydrofurane, 1,4-dioxan, n-hexane, cyclohexane, toluene, DME, dimethylsulfoxide in the presence of a base if needed, such as TMEDA, at a suitable temperature ranging from −78° C. to room temperature, for a time ranging from 15 minutes to 3 hours; the resulting lithium derivative can be quenched with a suitable electrophilic agent, such as trialkyl boronic esters, trialkylstannyl chloride, acid chlorides, acid fluorides, acid bromides, anhydrides, carbonates, halo carbonates, DMF, iodine, aldehydes, ketones, alkyl halides, in the presence of a suitable coordinating agent, such as ZnCl₂, ZnBr₂, CuCN.2LiCl, CuI, CuBr, CuBr.SMe₂ when needed, at a suitable temperature ranging from about −78° C. to reflux, for a time ranging from 15 minutes to about 72 hours.

According to step c) of the process, a compound of formula (I) wherein R is an optionally substituted aryl or C₁-C₆ alkenyl group and R₁, R₂, Rₐ, R_{b}, R_{c}, R_{d}, m and n are as defined above, can be obtained by reacting a compound of formula (I) wherein R is B(OR′″)₂, SnR″″, and R₁, R₂, Rₐ, R_{b}, R_{c}, R_{d}, R′″, R″″, m and n are as defined above, with a suitable aryl halide or halogeno olefine, in the presence of a suitable catalysing agent such as as palladium(0)tetrakis, bis triphenylphosphine palladium(II) dichloride, bis tricyclohexylphosphine palladium(II) dichloride, bis tri-o-tolylphosphine palladium(II) dichloride, palladium(II) acetate, tris(dibenzylideneacetone)dipalladium(0), [1,1′-bis(diphenylphosphino)ferrocene]dichloropalladium(II), [1,1′-bis(diphenylphosphino)ferrocene]dichloronickel(II), 1,4bis(diphenylphosphino)butane palladium(II), as sodium carbonate, cesium carbonate, potassium carbonate, potassium phosphate, triethylamine, sodium hydroxide, cesium fluoride, potassium tert-butylate, sodium ethylate, potassium acetate, in a suitable solvent, such as 1,4-dioxan, tetrahydrofurane, DMF, dimethoxyethane, toluene, methanol, ethanol, water, N-methylpyrrolidone and, if needed, adding a suitable ligand, such as tributylphosphine, triphenylphosphine, tri-o-tolylphosphine, tricyclohexyl, biphenyl(dicyclohexyl)phosphine, biphenyl(ditert-butyl)phosphine, diphenylphosphineferrocene, and/or a suitable Cu(I) salts, such as CuI, Cu(I)thiophene-2-carboxylate at a temperature ranging from room temperature to reflux, for a suitable time ranging from 15 minutes to 72 hours.

According to step c) of the process, a compound of formula (I) wherein R is an optionally substituted C₂-C₆ alkynyl, and R₁, R₂, Rₐ, R_{b}, R_{c}, R_{d}, m and n are as defined above, can be obtained by reacting a compound of formula (I) wherein R is B(OR′″)₂, SnR″″, and R₁, R₂, Rₐ, R_{b}, R_{c}, R_{d}, R′″, R″″, m and n are as defined above, with a suitable 1-alkyl(aryl)thio-alkyne, 1-iodo(bromo)alkyne, or 1,1-dibromo-1-alkene, in the presence of a suitable catalysing agent such as as palladium(0)tetrakis, bis triphenylphosphine palladium(II) dichloride, bis tricyclohexylphosphine palladium(II) dichloride, bis tri-o-tolylphosphine palladium(II) dichloride, palladium(II) acetate, tris(dibenzylideneacetone)dipalladium(0), [1,1′-bis(diphenylphosphino)ferrocene]dichloropalladium(II), [1,1′-bis(diphenylphosphino)ferrocene]dichloronickel(E), 1,4-bis(diphenylphosphino)butane palladium(II) in a suitable solvent, such as 1,4-dioxan, tetrahydrofurane, DMF, dimethoxyethane, toluene, methanol, ethanol, water, N-methylpyrrolidone and, if needed, adding a suitable ligand, such as tributylphosphine, triphenylphosphine, tri-o-tolylphosphine, tricyclohexyl, biphenyl(dicyclohexyl)phosphine, biphenyl(ditert-butyl)phosphine, diphenylphosphineferrocene, and/or a suitable Cu(I) salts, such as CuI, Cu(I)thiophene-2-carboxylate at a temperature ranging from room temperature to reflux, for a suitable time ranging from 15 minutes to 72 hours.

According to steps P and Pa of the process, a compound of formula (III) wherein R, Rₐ, R_{b}, R_{c}, R_{d}, m and n are as described above, R₁ is as described above but not hydrogen and Q is a solid support can be obtained by reacting a compound of formula (I) wherein R, Rₐ, R_{b}, R_{c}, R_{d}, m and n are as described above, R₁ is as described above but not hydrogen and R₂ is hydrogen (step P) or different from hydrogen (step Pa), with a suitable solid support such as a polymeric support like isocyanate polystyrenic resin, 2-chloro-trityl chloride resin, trityl chloride resin, p-nitrophenyl carbonate Wang resin, bromo-4-methoxyphenyl)methyl polystyrene or the like, which are all conventionally known in this field, in the presence, when needed, of a suitable base, such as diisopropylethylamine, triethylamine, 1,8-diazabiciclo[5.4.0]undec-7-ene or 2-tert-buytlimino-2-diethylamino-1,3-dimethylperhydro -1,3,2-diaza-phosphorine, in a suitable solvent such as dichloromethane, chloroform, tetrahydrofurane, dimethylformamide, dimethylacetamide, 1-methyl-2-pyrrolidinone, dimethylsulfoxide and the like, at a temperature ranging from room temperature to 50° C., for a suitable time ranging from 10 minutes to 90 hours.

According to step b1 a) of the process, a compound of formula (I) may be converted into a different compound of formula (I) by steps analogous to the steps b1) herein described for the conversion of a compound of the formula (I) into a different compound of formula (I).

According to step B of the process, a compound of formula (III) may be converted into a different compound of formula (III) by steps analogous to the steps b1), b2), c) and d) herein described for the conversion of a compound of the formula (I) into a different compound of formula (I).

According to step D of the process, a compound of formula (I) wherein R, Rₐ, R_{b}, R_{c}, R_{d}, m and n are as described above, R₁ is as described above and R₂ is hydrogen, can be obtained by cleaving a compound (III) wherein R, Rₐ, R_{b}, R_{c}, R_{d}, m and n are as described above, R₁ is as described above and Q is a solid support, according to conventional hydrolytic methods in the presence of a suitable acid, such as hydrochloric acid, acetic acid, trifluoroacetic acid, hydrofluoric acid, or in the presence of a suitable base, such as sodium hydroxide, potassium hydroxide, sodium carbonate, sodium hydrogencarbonate, piperidine, or in the presence of other hydrolytic agents, such as tetrabutyl ammoniumfluoride, trimethyl silylchloride, in a suitable solvent such as dichloromethane, chloroform, methanol, ethanol, trifluoroethanol, dioxan, at a temperature ranging from room temperature to 70° C., for a suitable time ranging from 10 minutes to 90 hours. R₂ is According to step E of the process, a compound of formula (I) wherein R, Rₐ, R_{b}, R_{c}, R_{d}, m and n are as described above, R₁ is as described above and R₂ is hydrogen may be converted into another different compound of formula (I), the conversion being carried out in several ways, depending on the meanings of the substituents and the presence of other substituents in the molecule. For example, by this conversion a compound of formula (I) wherein R₂ is as defined above but not hydrogen may be obtained.

According to step d) of the process, the conversion of a compound of formula (I) into another different compound of formula (I) may be carried out in several ways, depending on the meanings of the substituents and the presence of other substituents in the molecule. For example, a conversion can be a hydrolysis, a reductive amination, an arylation, an alkylation, an amination, a nucleophilic substitution, a catalytic reduction, an oxidation, a reduction, a condensation with an appropriate reagent or a combination of these reactions.

As an example, the compounds of formula (I) or (III), wherein R₁ is —COO^{t}Bu can be hydrolized to the corresponding compounds of formula (I) wherein R₁ is H, by treatment with a suitable acid, for instance trifluoroacetic or hydrochloric acid

So far, any of the above compounds of formula (I) or (III) wherein R₁ is a hydrogen atom can be easily converted into the corresponding derivatives alkylated, acylated, sulfonated or arylated. The reactions are carried out according to conventional techniques, for instance by properly reacting the amino derivative (1) or (III) wherein R₁ is hydrogen with alkylating, acylating, sulfonylating or arylating agents and the like.

In particular, a compound of formula (I) or (III) wherein R₁ is selected from R′ other than hydrogen, —COR′, —COOR′, —CONR′R″, —SO₂R′, or —SO₂NR′R″, wherein R′ and R″ have the above reported meanings; R, R₂ and Rₐ, R_{b}, R_{c}, R_{d}, m and n are as above defined, may be prepared by reacting a compound of formula (I) or a compound of formula (III), having R₁ equal to hydrogen, with a compound of formula (IV)
R₁—X (IV)

wherein R₁ is as above defined but not hydrogen and X is a suitable leaving group, preferably fluorine, chlorine, bromine or iodine.

The above reaction can be carried out according to conventional procedures well known in the art for acylating, sulfonylating, alkylating or arylating amino groups, for instance in the presence of a suitable base, such as potassium carbonate, triethylamine, N,N-diisopropylethylamine or pyridine, in a suitable solvent such as dimethylsulfoxide, toluene, dichloromethane, chloroform, diethyl ether, tetrahydrofurane, acetonitrile, or N,N-dimethylformamide, at a temperature ranging from about −10° C. to reflux and for a time varying from about 30 minutes to about 96 hours.

A compound of formula (I) or (III) wherein R₁ is an aryl group, R, R₂ and Rₐ, R_{b}, R_{c}, R_{d}, m and n are as above defined, may be prepared by reacting a compound of formula (I) or a compound of formula (III), having R₁ equal to hydrogen with a compound of formula (V)
R₁—X (V)

wherein R₁ is an aryl group and X is as above defined. The above reaction can be carried out according to conventional procedures well known in the art for arylating amino groups, for instance in the presence of a suitable catalyst when needed, such as palladium(0)tetrakis, bistriphenylphosphinePalladium(II)chloride, bis tricyclohexylphosphine palladium(II) dichloride, bis tri-o-tolylphosphine palladium(II) dichloride, palladium(II) acetate, tris(dibenzylideneacetone)dipalladium(0), [1,1′-bis(diphenylphosphino) ferrocene]dichloropalladium(II), as sodium carbonate, cesium carbonate, potassium carbonate, potassium phosphate, triethylamine, sodium hydroxide, cesium fluoride, potassium tert-butylate, sodium tert-butylate, sodium ethylate, potassium acetate, in a suitable solvent, such as 1,4dioxan, tetrahydrofurane, DMF, dimethilsulfoxide, dimethoxyethane, toluene, methanol, ethanol, water, N-methylpyrrolidone and adding a suitable ligand, such as tributylphosphine, triphenylphosphine, tri-o-tolylphosphine, tricyclohexyl, biphenyl(dicyclohexyl)phosphine, biphenyl(ditert-butyl)phosphine, diphenylphosphineferrocene, BINAP [(2,2′-bis(diphenylphosphino)-1,1′-binaphthyl], and adding, when needed a phase transfer catalysing agent, such as 18-crown-6, at a temperature ranging from room temperature to reflux, for a suitable time ranging from 15 minutes to 72 hours.

From the foregoing it is clear to the person skilled in the art that the preparation of the compounds of formula (I) or (III) having R₁ equal to —SO₂NR′R″ can be actually performed as above described or, alternatively, by properly reacting a compound of formula (I) or (III) having R₁ equal to —SO₂NHR′ with any suitable alkylating moiety, according to well known methodologies for preparing di-substituted sulfonamides.

A compound of formula (I) or (III) wherein R₁ is a —CONHR′ group, R′ has the above reported meanings other than hydrogen, R, R₂, and Rₐ, R_{b}, R_{c}, R_{d}, m and n are as above defined, may be prepared by reacting a compound of formula (I) or a compound of formula (III) having R₁ equal to hydrogen, with a compound of formula (VI)
R′—NCO (VI)

wherein R′ is as above defined but not hydrogen, so as to obtain a corresponding compound of formula (I) or (III) which may be optionally further reacted with a compound of formula (VII)
R″—X (VII)

wherein R″ is as above defined other than hydrogen and X is as above defined, so as to obtain a compound of formula (I) or (III) wherein R₁ is —CONR′R″, wherein R′ and R″ are as above defined but not hydrogen atom.

The reaction between the above compounds (I) or (III) with a compound of formula (VII) can be carried out in the presence of a tertiary base, such as triethylamine, N,N-diisopropylethylamine or pyridine, in a suitable solvent, such as toluene, dichloromethane, chloroform, diethyl ether, tetrahydrofurane, acetonitrile, or N,N-dimethylformamide, at a temperature ranging from about −10° C. to reflux and for a time varying from about 30 minutes to about 72 hours.

The optional subsequent conversion of a compound of formula (I) or (III) having R₁ equal to —CONHR′ into a corresponding derivative having R₁ equal to —CONR′R″ is carried out according to conventional methods used to prepare di-substituted ureido derivatives.

A compound of formula (I) or (III) wherein R₁ is a —CONR′R″ group, R′ and R″ has the above reported meanings other than hydrogen, R, R₂ and Rₐ, R_{b}, R_{c}, R_{d}, m and n are as above defined, may be prepared by reacting a compound of formula (I) or a compound of formula (III) having R₁ equal to hydrogen with 4-nitrophenylchloroformate and subsequently with a compound of formula (VIII)
R′R″NH (VIII)

wherein R′ and R″ are as defined above but not hydrogen.

The reaction is carried out according to conventional methods used to prepare di-substituted ureido derivatives.

Alternatively, a compound of formula (I) or a compound of formula (III), having R₁ equal to hydrogen may be reacted under reductive conditions with a compound of formula (IX)
R′—CHO (IX)

wherein R′ is as defined above but not hydrogen, so as to obtain a corresponding compound of formula (I) or (III) wherein R₁ is a —CH₂R′ group and R′ being as defined above but not hydrogen.

The reaction is carried out in a suitable solvent such as, for instance, N,N-dimethylformamide, N,N-dimethylacetamide, chloroform, dichloromethane, tetrahydrofurane, or acetonitrile, optionally in the presence of acetic acid, ethanol or methanol as co-solvents, at a temperature ranging from about −10° C. to reflux and for a time varying from about 30 min to about 4 days.

Conventional reducing agents in the reaction medium are, for instance, sodium boron hydride, sodium triacethoxy boron hydride, and the like.

In a further example, any of the above compounds of formula (I) or of formula (III) wherein one or more of Rₐ, R_{b}, R_{b} and R_{d} is —CH₂OH may be conveniently prepared by starting from a corresponding protected derivative having one or more of Rₐ, R_{b}, R_{b} and R_{d} as —CH₂—O—Si(Me)₂tBu or —CH₂—O—Ph.

The reaction is carried according to conventional techniques, for instance in a suitable solvent such as, for instance, N,N-dimethylformamide, chloroform, dichloromethane, tetrahydrofurane, methanol, ethanol or acetonitrile, at a temperature ranging from about −10° C. to reflux and for a time varying from about 30 min to about 72 hours with a suitable fluoride source, for instance tetrabutylamonium fluoride.

Likewise, the above compounds of formula (I) or (III) having one or more Rₐ, R_{b}, R_{c} and R_{d} equal to —CH₂OH can be reacted with a compound of formula (VII′)
R′—X (VII′)

wherein R′ is as above defined but not hydrogen and X is as above defined, so as to obtain the corresponding compounds wherein one or more Rₐ, R_{b}, R_{c} and R_{d} are a —CH₂OR′ group, wherein R′ is as defined above but not hydrogen.

This latter reaction can be carried out in the presence of a base, such as sodium hydride, N,N-diisopropylethylamine or pyridine, in a suitable solvent, such as toluene, dichloromethane, chloroform, diethyl ether, tetrahydrofurane, acetonitrile, or N,N-dimethylformamide, at a temperature ranging from about −10° C. to reflux.

In an analogous manner, a compound of the formula I wherein R₂ is hydrogen may be converted into another compound of the formula I wherein R₂ is as defined above but not hydrogen atom.

The starting compound of formula (II) are known or can be prepared starting from known compounds using known methods of preparation, for example those described in WO02/12242. As it will be really appreciated by the man skilled in the art, when preparing the compounds of formula (I) object of the invention, optional functional groups within both the starting materials or the intermediates thereof, which could give rise to unwanted side reactions, need to be properly protected according to conventional techniques. Likewise, the conversion of these latter into the free deprotected compounds may be carried out according to known procedures.

The above cited reagents of the process, i.e. arylboronic acids, arylboronic esters, alkenylboronic acids, alkenylboronic esters, triarylstannanes, acid chlorides, acid fluorides, acid bromides, anhydrides, carbonates, halo carbonates, alkynes, aryl halides, halogeno alkenes and the compounds of formula (IV), (V), (VI), (VII), (VII′), (VIII) and (IX) are known or can be prepared according to known methods.

As it will be also really appreciated by the man skilled in the art, when preparing the compounds of formula (I) object of the invention, according to steps a)-c), each of the above cited reactants can be replaced by the corresponding polymer-supported reactant.

In addition to the above, it is also clear to the skilled man that the compounds of formula (I) of the invention can be advantageously prepared by combining the above described reactions in a combinatorial fashion, for example according to solid-phase-synthesis (SPS) techniques, so as to get a combinatorial chemical library of compounds of formula (I).

It is therefore a further object of the invention a library of two or more compounds of formula (I):

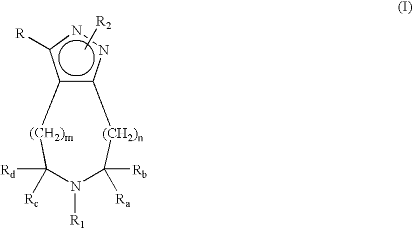

wherein R, R₁, R₂ Rₐ, R_{b}, R_{c}, R_{d} m and n are as defined above, which can be obtained starting from one or more compound supported onto a solid support of the formula (III) as defined above.

Pharmacology

The compounds of formula (I) are active as protein kinase inhibitors and are therefore useful, for instance, to restrict the unregulated proliferation of tumor cells.

In therapy, they may be used in the treatment of various tumors, such as those formerly reported, as well as in the treatment of other cell proliferative disorders such as psoriasis, vascular smooth cell proliferation associated with atherosclerosis and post-surgical stenosis and restenosis and in the treatment of Alzheimer's disease.

The inhibiting activity of putative cdk/cyclin inhibitors and the potency of selected compounds is determined through a method of assay based on the use of the SPA technology (Amersham Pharmacia Biotech).

The assay consists of the transfer of radioactivity labelled phosphate moiety by the kinase to a biotinylated substrate. The resulting 33P-labelled biotinylated product is allowed to bind to streptavidin-coated SPA beads (biotin capacity 130 pmol/mg), and light emitted was measured in a scintillation counter.

Inhibition Assay of Cdk2/Cyclin A Activity

Kinase reaction: 4 μM in house biotinylated histone H1 (Sigma #H-5505) substrate, 10 μM ATP (0.1 microCi P³³γ-ATP), 1.1 nM Cyclin A/CDK2 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After incubation for 60 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μL of 5M CsCl. After 4 hours, the plates were read for 2 min in a Packard TOP-Count radioactivity reader.

IC50 determination: inhibitors were tested at different concentrations ranging from 0.0015 to 10 μM. Experimental data were analyzed by the computer program GraphPad Prizm using the four parameter logistic equation:
y=bottom+(top-bottom)/(1+10ˆ((log IC50-x)*slope))

where x is the logarithm of the inhibitor concentration, y is the response; y starts at bottom and goes to top with a sigmoid shape.

Ki Calculation:

Experimental method: Reaction was carried out in buffer (10 mM Tris, pH 7.5, 10 mM MgCl₂, 0.2 mg/ml BSA, 7.5 mM DTT) containing 3.7 nM enzyme, histone and ATP (constant ratio of cold/labeled ATP 1/3000). Reaction was stopped with EDTA and the substrate captured on phosphomembrane (Multiscreen 96 well plates from Millipore).

After extensive washing, the multiscreen plates were read on a top counter. Control (time zero) for each ATP and histone concentrations was measured.

Experimental design: Reaction velocities are measured at four ATP, substrate (histone) and inhibitor concentrations. An 80-point concentration matrix was designed around the respective ATP and substrate Km values, and the inhibitor IC50 values (0.3, 1, 3, 9 fold the Km or IC50 values). A preliminary time course experiment in the absence of inhibitor and at the different ATP and substrate concentrations allows the selection of a single endpoint time (10 min) in the linear range of the reaction for the Ki determination experiment.

Kinetic parameter estimates: Kinetic parameters were estimated by simultaneous nonlinear least-square regression using [Eq.1] (competitive inhibitor respect to ATP, random mechanism) using the complete data set (80 points):

$$v = \frac{Vm \cdot A \cdot B}{\begin{matrix}α \cdot Ka \cdot Kb + α \cdot Ka \cdot B + a \cdot Kb \cdot \\ A + A \cdot B + α \cdot \frac{Ka}{Ki} \cdot I \cdot ( Kb + \frac{B}{β} )\end{matrix}} \qquad [ Eq . 1 ]$$

where A=[ATP], B=[Substrate], I=[inhibitor], Vm=maximum velocity, Ka, Kb, Ki the dissociation constants of ATP, substrate and inhibitor respectively. α and β the cooperativity factor between substrate and ATP binding and substrate and inhibitor binding respectively.

In addition the selected compounds are characterized on a panel of ser/thre kinases strictly related to cell cycle (cdk2/cyclin E, cdk1/cyclin B1, cdk5/p25, cdk4/cyclin D1), and also for specificity on MAPK, PKA, EGFR, IGF1-R, Aurora-2 and Cdc 7

Inhibition Assay of Cdk2/Cyclin E Activity

Kinase reaction: 10 μM in house biotinylated histone H1 (Sigma #H-5505) substrate, 30 μM ATP (0.3 microCi P³³γ-ATP), 4 ng GST-Cyclin E/CDK2 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After incubation for 60 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96well OPTIPLATEs containing 100 μl of 5M CsCl. After 4 hours, the plates were read for 2 min in a Packard TOP-Count radioactivity reader.

IC50 determination: see above

Inhibition Assay of Cdk1/Cyclin B1 Activity

Kinase reaction: 4 μM in house biotinylated histone H1 (Sigma #H-5505) substrate, 20 μM ATP (0.2 microCi P³³γ-ATP), 3 ng Cyclin B/CDK1 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 20 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate. After 20 min. incubation for substrate capture, 100 μl 5M CsCl were added to allow statification of beads to the top of the Optiplate and let stand 4 hours before radioactivity counting in the Top-Count instrument.

IC50 determination: see above

Inhibition Assay of Cdk5/p25 Activity

The inhibition assay of cdk5/p25 activity is performed according to the following protocol.

Kinase reaction: 10 μM biotinylated histone H1 (Sigma #H-5505) substrate, 30 μM ATP (0.3 microCi P33γ-ATP), 15 ng CDK5/p25 complex, inhibitor in a final volume of 30 μl buffer (TRIS HCl 10 mM pH 7.5, MgCl2 10 nM, DTT 7.5 mM+0.2 mg/ml BSA) were added to each well of a 96 U bottom. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 determination: see above

Inhibition Assay of Cdk4/Cyclin D1 Activity

Kinase reaction: 0,4 μM μM mouse GST-Rb (769-921) (#sc-4112 from Santa Cruz) substrate, 10 μM ATP (0.5 μCi P³³γ-ATP), 100 ng of baculovirus expressed GST-cdk4/GST-Cyclin D1, suitable concentrations of inhibitor in a final volume of 50 μl buffer (TRIS HCl 10 mM pH 7.5, MgCl₂ 10 mM, 7.5 mM DTT+0.2 mg/ml BSA) were added to each well of a 96 U bottom well plate. After 40 min at 37° C. incubation, reaction was stopped by 20 μl EDTA 120 mM.

Capture: 60 μl were transferred from each well to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection: filters were allowed to dry at 37° C., then 100 μl/well scintillant were added and ³³P labeled Rb fragment was detected by radioactivity counting in the Top-Count instrument.

IC50 determination: see above

Inhibition Assay of MAPK Activity

Kinase reaction: 10 μM in house biotinylated MBP (Sigma #M-1891) substrate, 15 μM ATP (0.15 microCi P³³γ-ATP), 30 ng GST-MAPK (Upstate Biothecnology #14-173), inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 determination: see above

Inhibition Assay of PKA Activity

Kinase reaction: 10 μM in house biotinylated histone H1 (Sigma #H-5505) substrate, 10 μM ATP (0.2 microM P³γ-ATP), 0.45 U PKA (Sigma #2645), inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After incubation for 90 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTlPLATEs containing 100 μl of 5M CsCl. After 4 hours, the plates were read for 2 min in a Packard TOP-Count radioactivity reader.

IC50 determination: see above

Inhibition Assay of EGFR Activity

Kinase reaction: 10 μM in house biotinylated MBP (Sigma #M-1891) substrate, 2 μM ATP (0.04 microCi P³³γ-ATP), 36 ng insect cell expressed GST-EGFR, inhibitor in a final volume of 30 μl buffer (Hepes 50 mM pH 7.5, MgCl₂ 3 mM, MnCl₂ 3 mM, DTT 1 mM, NaVO₃ 3 μM,+0.2 mg/ml BSA) were added to each well of a 96 U bottom. After incubation for 20 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 determination: see above

Inhibition Assay of IGF1-R Activity

The inhibition assay of IGF1-R activity is performed according to the following protocol.

Enzyme activation: IGF1-R must be activated by auto-phosphorylation before starting the experiment. Just prior to the assay, a concentrated enzyme solution (694 nM) is incubated for half a hour at 28° C. in the presence of 100 μM ATP and then brought to the working dilution in the indicated buffer.

Kinase reaction: 10 μM biotinylated IRS1 peptide (PRIMM) substrate, 0-20 μM inhibitor, 6 μM ATP, 1 microCi ³³P-ATP, and 6 nM GST-IGF1-R (pre-incubated for 30 min at room temperature with cold 60 μM cold ATP) in a final volume of 30 μl buffer (50 mM HEPES pH 7.9, 3 MM MnCl₂, 1 mM DTT, 3 μl NaVO₃) were added to each well of a 96 U bottom well plate. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

Inhibition Assay of Aurora-2 Activity

Kinase reaction: 8 μM biotinylated peptide (4 repeats of LRRWSLG), 10 μM ATP (0.5 uCi P³³γ-ATP), 7.5 ng Aurora 2, inhibitor in a final volume of 30 μl buffer (HEPES 50 mM pH 7.0, MgCl₂ 10 mM, 1 mM DTT, 0.2 mg/ml BSA, 3 μM orthovanadate) were added to each well of a 96 U bottom well plate. After 60 minutes at room temperature incubation, reaction was stopped and biotinylated peptide captured by adding 100 μl of bead suspension.

Stratification: 100 μl of CsCl2 5 M were added to each well and let stand 4 hour before radioactivity was counted in the Top-Count instrument.

IC50 determination: see above

Inhibition Assay of Cdc7/dbf4 Activity

The inhibition assay of Cdc7/dbf4 activity is performed according to the following protocol.

The Biotin-MCM2 substrate is trans-phosphorylated by the Cdc7/Dbf4 complex in the presence of ATP traced with γ³³-ATP. The phosphorylated Biotin-MCM2 substrate is then captured by Streptavidin-coated SPA beads and the extent of phosphorylation evaluated by β counting.

The inhibition assay of Cdc7/dbf4 activity was performed in 96 wells plate according to the following protocol.

To each well of the plate were added:
10 μl substrate (biotinylated MCM2, 6 μM final concentration)
10 μl enzyme (Cdc7/Dbf4, 17.9 nM final concentration)
10 μl test compound (12 increasing concentrations in the nM to μM range to generate a dose-response curve)
10 μl of a mixture of cold ATP (2 μM final concentration) and radioactive ATP (1/5000 molar ratio with cold ATP) was then used to start the reaction which was allowed to take place at 37° C.

Substrate, enzyme and ATP were diluted in 50 mM HEPES pH 7.9 containing 15 mM MgCl₂, 2 mM DTT, 3 μM NaVO₃, 2 mM glycerophosphate and 0.2 mg/ml BSA. The solvent for test compounds also contained 10% DMSO.

After incubation for 60 minutes, the reaction was stopped by adding to each well 100 μl of PBS pH 7.4 containing 50 mM EDTA, 1 mM cold ATP, 0.1% Triton X100 and 10 mg/ml streptavidin coated SPA beads.

After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 determination: see above.

The compounds of formula (I) of the present invention, suitable for administration to a mammal, e.g. to humans, can be administered by the usual routes and the dosage level depends upon the age, weight, conditions of the patient and the administration route.

For example, a suitable dosage adopted for oral administration of a compound of formula (I) may range from about 10 to about 500 mg pro dose, from 1 to 5 times daily.

The compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or by intravenous and/or intrathecal and/or intraspinal injection or infusion.

In addition, the compounds of the invention can be administered either as single agents or, alternatively, ill combination with known anticancer treatments such as radiation therapy or chemotherapy regimen in combination with cytostatic or cytotoxic agents, antibiotic-type agents, alkylating agents, antimetabolite agents, hormonal agents, immunological agents, interferon-type agents, cyclooxygenase inhibitors (e.g. COX-2 inhibitors), metallomatrixprotease inhibitors, telomerase inhibitors, tyrosine kinase inhibitors, anti-growth factor receptor agents, anti-HER agents, anti-EGFR agents, anti-angiogenesis agents, farnesyl transferase inhibitors, ras-raf signal transduction pathway inhibitors, cell cycle inhibitors, other cdks inhibitors, tubulin binding agents, topoisomerase I inhibitors, topoisomerase II inhibitors, and the like.

As an example, the compounds of the invention can be administered in combination with one or more chemotherapeutic agents such as, for instance, exemestane, formestane, anastrozole, letrozole, fadrozole, taxane, taxane derivatives, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, tamoxifen, raloxifen, Sugen SU-5416, Sugen SU-6668, Herceptin, and the like, optionally within liposomal formulations thereof.

If formulated as a fixed dose, such combination products employ the compounds of this invention within the dosage range described above and the other pharmaceutically active agent within the approved dosage range.

Compounds of formula (I) may be used sequentially with known anticancer agents when a combination formulation is inappropriate.

It is therefore a further object of the invention a product or kit comprising the compound of formula (I) of the invention and one or more chemotherapeutic agents for simultaneous, separate or sequential use in anticancer therapy or for the treatment of cell proliferative disorders.

The present invention also includes pharmaceutical compositions comprising an effective amount of a compound of formula (I) or a pharmaceutically acceptable salt thereof in association with a pharmaceutically acceptable excipient, carrier or diluent.

The pharmaceutical compositions containing the compounds of the invention are usually prepared following conventional methods and are administered in a pharmaceutically suitable form.

For example, the solid oral forms may contain, together with the active compound, diluents, e.g. lactose, dextrose, saccharose, sucrose, cellulose, corn starch or potato starch; lubricants, e.g. silica, talc, stearic, magnesium or calcium stearate, and/or polyethylene glycols; binding agents, e.g. starches, arabic gum, gelatine, methylcellulose, carboxymethylcellulose or polyvinyl pyrrolidone; disaggregating agents, e.g. a starch, alginic, alginates or sodium starch glycolate; effervescing mixtures; dyestuffs; sweeteners; wetting agents such as lecithin, polysorbates, laurylsulphates; and, in general, non-toxic and pharmacologically inactive substances used in pharmaceutical formulations. Said pharmaceutical preparations may be manufactured in known manner, for example, by means of mixing, granulating, tabletting, sugar-coating, or film-coating processes.

The liquid dispersions for oral administration may be. e.g. syrups, emulsions and suspensions.

The syrups may contain as carrier, for example, saccharose or saccharose with glycerine and/or mannitol and/or sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as carrier, for example, sterile water or preferably they may be in the form of sterile, aqueous, isotonic saline solutions or they may contain as a carrier propylene glycol.

The suppositories may contain together with the active compound a pharmaceutically acceptable carrier, e.g. cocoa butter, polyethylene glycol, a polyoxyethylene sorbitan fatty ester surfactant or lecithin.

General Methods

The following examples illustrates the invention without limiting it.

HPLC Conditions

LCMS instrument comprising:
Hewlett Packard 1312A binary pump
Gilson 215 autosampler fitted with a 1 ml syringe
Polymer Labs PL1000 Evaporative Light Scattering Detector
Micromass ZMD mass spectrometer operating in Electrospray positive ionisation mode.

The LC eluent is split and approximately 200 μl/min enters the mass spectrometer, 800 μl/min to the ELS. The instruments are currently controlled using Micromass MassLynx 3.5 software under Windows NT4.0

HPLC Conditions

| Mobile Phase: | Aqueous - Water + 0.1% Trifluoroacetic acid | | |
|---|---|---|---|
| | Organic - Acetonitrile + 0.1% Trifluoroacetic acid | | |
| Gradient: | Time (mins) | % Aqueous | % Organic |
| | 0.0 | 100 | 0 |
| | 1.8 | 5 | 95 |
| | 2.1 | 5 | 95 |
| | 2.3 | 100 | 0 |
| | 2.4 | 100 | 0 |

| | |
|---|---|
| Run time: | 2.4 mins |
| Flow rate: | 1 ml/min |
| Injection vol: | 3 μl |
| Column temperature: | ambient (20° C.) |
| Column: | 50 × 2.0 mm Hypersil C18 BDS; 5 μm |
| ELS Detector | Nebuliser Temperature 8° C. |
| | Evaporation temperature 9° C. |
| | Gas Flow 1.5 l/hr |
| MS Detector | m/z 150-800 @ 0.5 secs/scan, 0.1 second |
| | interscan delay |
| | Cone voltage 25 V, Source Temp. 140° C. |
| | Drying Gas 350 l/hr |

As formerly indicated, several compounds of formula (I) of the invention have been synthesized in parallel, according to combinatorial chemistry techniques.

In this respect, some compounds thus prepared have been conveniently and unambiguously identified, as per the coding system of tables I-III, together with HPLC retention time and mass.

Each code, which identifies a single specific compound of formula (I), consists of three units A-M-B.

A represents any substituent R-[see formula (I)] and is directly attached to the rest of the pyrrolopyrazole moiety so as to get pyrrolopyrazole derivatives being substituted in position 3 (A-M-B); each A radical (substituent) is represented in the following table I.

B represents any substituent R₁-[see formula (I)] and is attached to the rest of the pyrrolopyrazole moiety through the nitrogen atom so as to get pyrrolopyrazole derivatives being substituted in position 5 (A-M-B); each B radical (substituent) is represented in the following table II.

M refers to the central core of the divalent pyrrolopyrazole moiety and is substituted by groups A and B.

For ease of reference, each A or B groups of tables I and II has been identified with the proper chemical formula also indicating the point of attachment with the rest of the molecule M.

Just as an example, the compound A7-M-B30 of table III (see entry 133) represents a pyrrolopyrazole M being substituted in position 3 (direct bond) by the group A7 and in position 5 (through the —N— group) by the group B30.

TABLE I

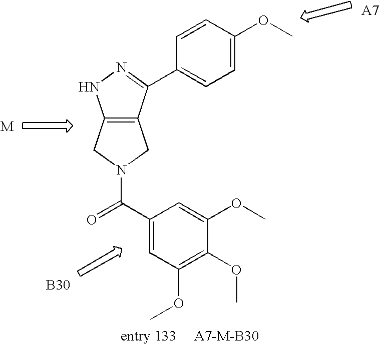

A group

| Code | Fragment |
|---|---|
| A1 | 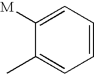 |
| A2 | 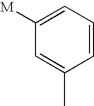 |
| A3 | 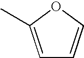 |
| A4 | 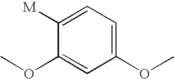 |
| A5 | 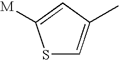 |
| A6 | 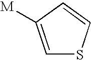 |
| A7 | 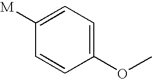 |
| A8 | 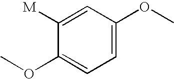 |
| A9 | 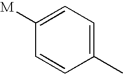 |
| A10 | 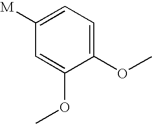 |
| A11 | 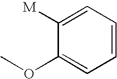 |
| A12 | 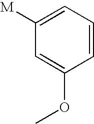 |
| A13 | 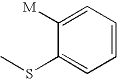 |
| A14 | 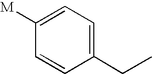 |
| A15 | 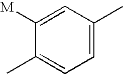 |
| A16 | 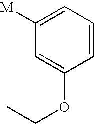 |
| A17 | 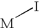 |
| A18 | 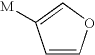 |
| A19 | 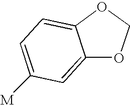 |
| A20 | 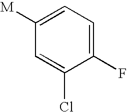 |
| A21 | 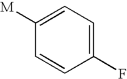 |
| A22 | 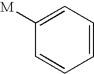 |
| A23 | 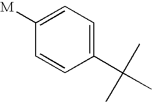 |
| A24 | 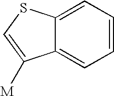 |
| A25 | 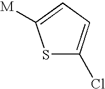 |
| A26 | 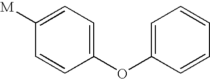 |
| A27 | 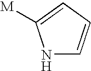 |
| A28 | 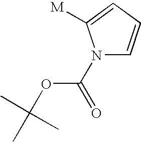 |
| A29 | 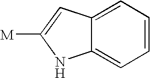 |
| A30 | 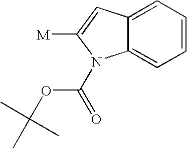 |

TABLE II
B groups

| Code | Fragment |
|---|---|
| B1 | 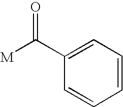 |
| B2 | 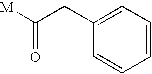 |
| B3 | 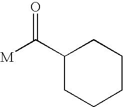 |
| B4 | 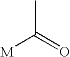 |
| B5 | 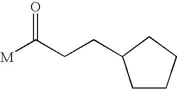 |
| B6 | 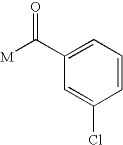 |
| B7 | 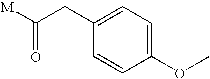 |
| B8 | 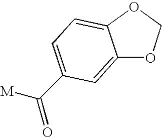 |
| B9 | 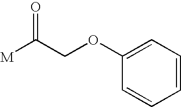 |
| B10 | 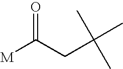 |
| B11 | 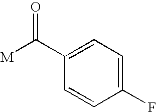 |
| B12 | 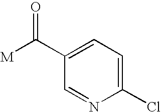 |
| B13 | 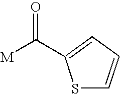 |
| B14 | 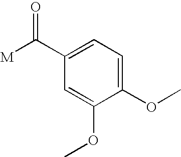 |
| B15 | 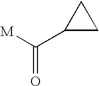 |
| B16 | 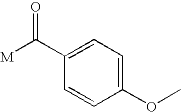 |
| B17 | 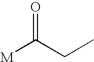 |
| B18 | 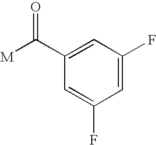 |
| B19 | 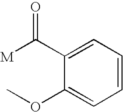 |
| B20 | 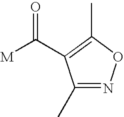 |
| B21 | 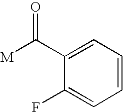 |
| B22 | 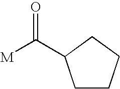 |
| B23 | 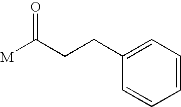 |
| B24 | 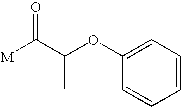 |
| B25 | 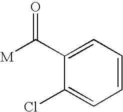 |
| B26 | 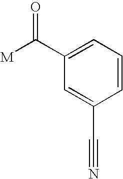 |
| B27 | 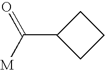 |
| B28 | 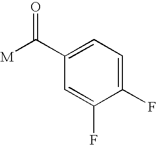 |
| B29 | 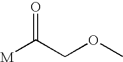 |
| B30 | 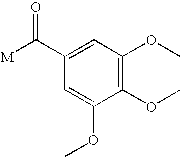 |
| B31 | 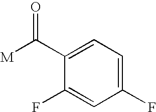 |
| B32 | 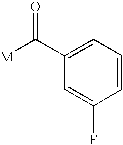 |
| B33 | 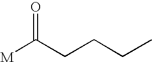 |
| B34 | 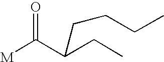 |
| B35 | 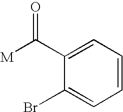 |
| B36 | 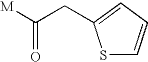 |
| B37 | 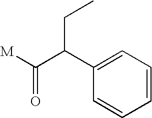 |
| B38 | 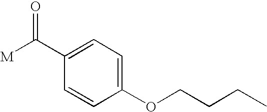 |
| B39 | 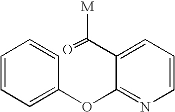 |
| B40 | 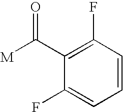 |
| B41 | 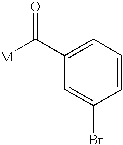 |
| B42 | 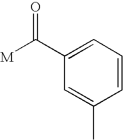 |
| B43 | 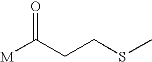 |
| B44 | 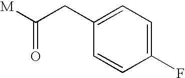 |
| B45 | 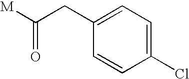 |
| B46 | 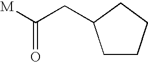 |
| B47 | 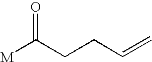 |
| B48 | 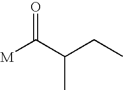 |
| B49 | 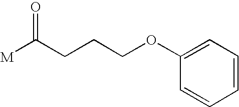 |
| B50 | 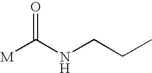 |
| B51 | 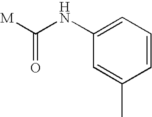 |
| B52 | 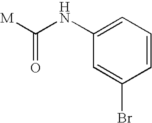 |
| B53 | 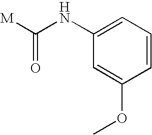 |
| B54 | 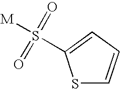 |
| B55 | 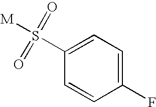 |
| B56 | 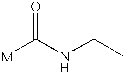 |
| B57 | 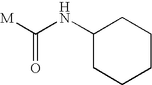 |
| B58 | 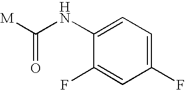 |
| B59 | 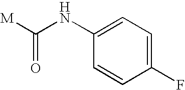 |
| B60 | 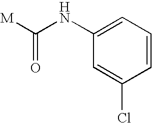 |
| B61 | 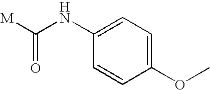 |
| B62 | 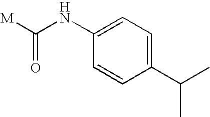 |
| B63 | 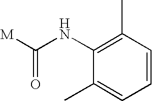 |
| B64 | 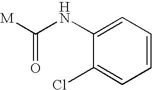 |
| B65 | 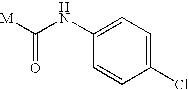 |
| B66 | 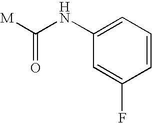 |
| B67 | 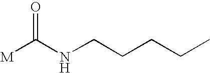 |
| B68 | 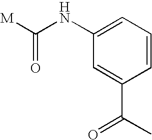 |
| B69 | 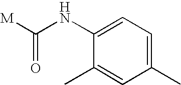 |
| B70 | 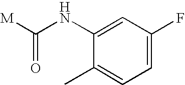 |
| B71 | 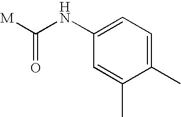 |
| B72 | 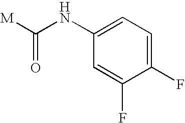 |
| B73 | 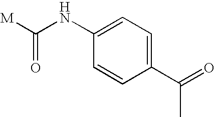 |
| B74 | 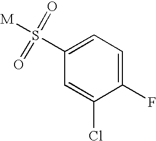 |
| B75 | 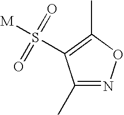 |
| B76 | 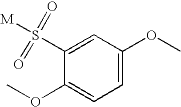 |
| B77 | 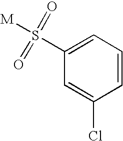 |
| B78 | 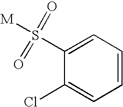 |
| B79 | 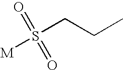 |
| B80 | 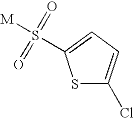 |
| B81 | 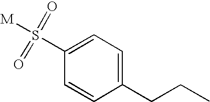 |
| B82 | 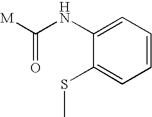 |
| B83 | 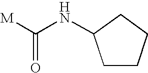 |
| B84 | 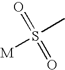 |
| B85 | 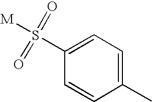 |
| B86 | 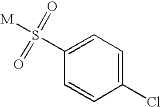 |
| B87 | 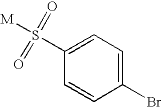 |
| B88 | 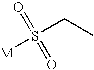 |
| B89 | 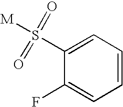 |
| B90 | 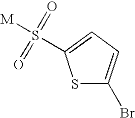 |
| B91 | 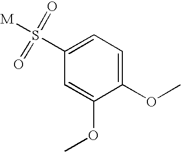 |
| B92 | 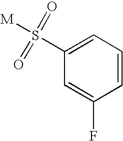 |
| B93 | 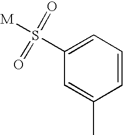 |
| B94 | 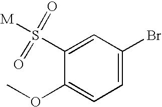 |
| B95 | 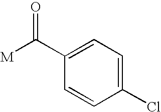 |
| B96 | 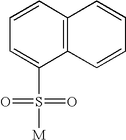 |
| B97 | 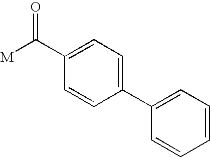 |
| B98 | 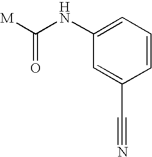 |
| B99 | 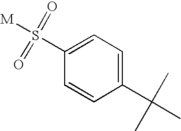 |
| B100 | 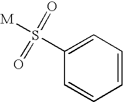 |
| B101 | 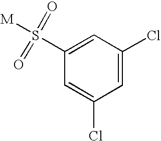 |
| B102 | 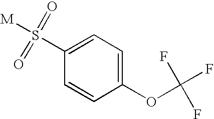 |
| B103 | 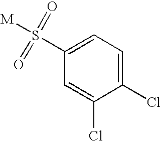 |
| B104 | 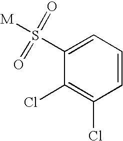 |
| B105 | 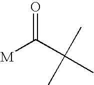 |
| B106 | 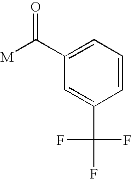 |
| B107 | 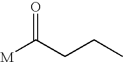 |
| B108 | 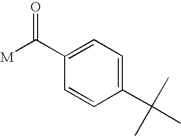 |
| B109 | 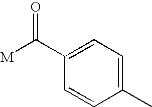 |
| B110 | 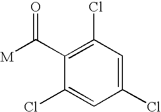 |
| B111 | 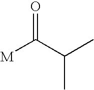 |
| B112 | 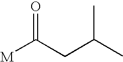 |
| B113 | 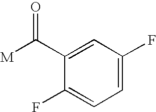 |
| B114 | 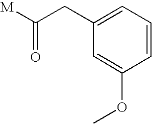 |
| B115 | 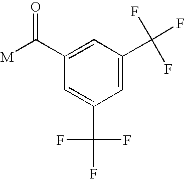 |
| B116 | 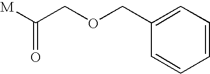 |
| B117 | 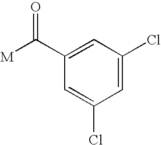 |
| B118 | 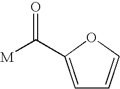 |
| B119 | 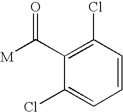 |
| B120 | 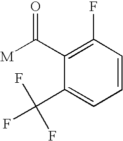 |
| B121 | 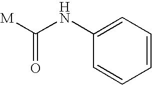 |
| B122 | 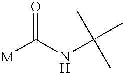 |
| B123 | 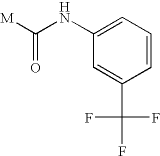 |
| B124 | 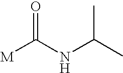 |
| B125 | 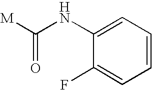 |
| B126 | 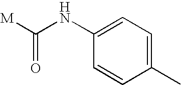 |
| B127 | 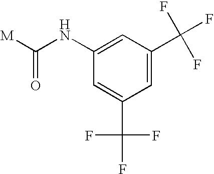 |
| B128 | 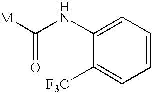 |
| B129 | 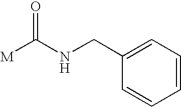 |
| B130 | 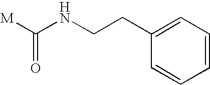 |
| B131 | 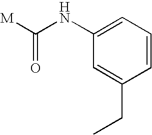 |
| B132 | 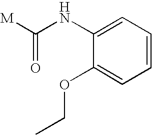 |
| B133 | 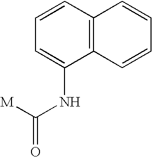 |
| B134 | 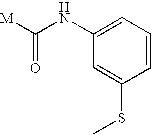 |
| B135 | 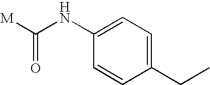 |
| B136 | 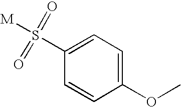 |
| B137 | 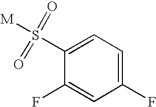 |
| B138 | 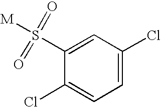 |
| B139 | 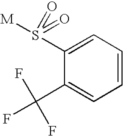 |
| B140 | 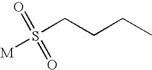 |
| B141 | 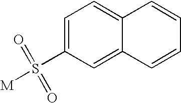 |
| B142 | 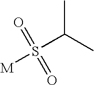 |
| B143 | 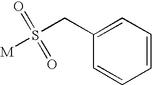 |
| B144 | 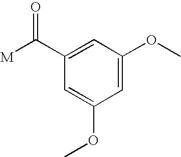 |
| B145 | 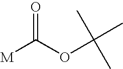 |

### EXAMPLE 1

Preparation of 5-tert-butyloxycarbonyl-1-ethoxycarbonyl-4,6-dihydropyrrolo[3,4-c]pyrazole (I, Rₐ═R_{b}═R_{c}═R_{d}═H, R═H, R₁=t-Butyloxycarbonyl(BOC), R₂=ethoxycarbonyl).

A solution of 3-amino-5-tert-butyloxycarbonyl-1-ethoxycarbonyl-4,6-dihydropyrrolo[3,4-c]pyrazole (0.4 g, 1.35 mmol) in dry tetrahydrofurane (10 ml) was added drop wise to a solution of isoamylnitrite (0.32 ml, 2.36 mmol) in dry tetrahydrofurane (2 ml) maintained at reflux. The resulting solution was stirred at reflux for 4 hours, and then cooled to room temperature. After removal of the solvent under vacuum, the crude material was purified by flash chromatography on silica gel using n-hexane÷ethyl acetate 90÷10; 70÷30. The title compound was obtained as a light yellow oil (200 mg, y 53%).

¹H-NMR(DMSO-d₆) δ ppm: 7.67(s, 1H); 4.54(m, 2H); 4.39(q,2H); 4.32(m, 2H); 1.43(s,9H); 1.31 (t,3H).

Operating in an analogous way, the following compound was also obtained 5-tert-butyloxycarbonyl-2-ethoxycarbonyl-4,6-dihydropyrrolo[3,4-c]pyrazole

¹H-NMR(DMSO-d₆) δ ppm: 8.05(s, 1H); 4.39(q,2H); 4.37(m, 4H); 1.43(s,9H); 1.31(t,3H).

### EXAMPLE 2

Preparation of 5-tert-butyloxycarbonyl-1(2)H-4,6-dihydropyrrolo[3,4-c]pyrazole (I, Rₐ═R_{b}═R_{c}═R_{d}═H, R₁═H, R₁=t-Butyloxycarbonyl(BOC), R₂═H).

5-tert-butyloxycarbonyl-1-ethoxycarbonyl-4,6-dihydropyrrolo[3,4-c]pyrazole (1.5 g, 5.3 mmol) was treated with a solution of 10% triethylamine in methanol (74 ml) at room temperature for about 20 hours. After removal of the solvents under vacuum, the crude material was dissolved with chloroform (30 ml) and washed with water (20 ml×2), brine (20 ml), dried over sodium sulphate, filtered and evaporated to dryness. The title compound was obtained as a beige powder (1.08 g, yield 97%).

¹H-NMR (DMSO-d₆) δ ppm: 12.63(s,1H); 7.47(s, 1H); 4.31(m, 4H); 1.42(s,9H).

Operating in an analogous way, the following compounds were obtained:

3-iodo-5-t-butyloxycarbonyl-1(2)H-4,6-dihydropyrrolo[3,4-c]pyrazole (I, Rₐ═R_{b}═R_{c}═R_{d}═H, R═I, R₁=t-butyloxycarbonyl, R₂═H).

¹H-NMR (CDCl₃) δ ppm: 11.00 (1H, br. s), 4.60-4.26 (4H, m), 1.46 (9H, s)

3-iodo-5-isopropylaminocarbonyl-1(2)H-4,6-dihydropyrrolo[3,4-c]pyrazole (I, Rₐ═R_{b}═R_{c}═R_{d}═H, R═I, R₁=3-isopropylaminocarbonyl, R₂═H).

¹H-NMR (DMSO-d₆) δ ppm: 13.03(s,1H); 5.63(s, 1H); 4.18(m, 4H); 3.78(m, 1H); 1.07(d, 6H).

### EXAMPLE 3

Preparation of 5-tert-butyloxycarbonyl-1-(2-trimethylsilanyl-ethyloxymethyl)-4,6-dihydropyrrolo[3,4-c]pyrazole and 5-tert-butyloxycarbonyl-2-(2-trimethylsilanyl-ethyloxymethyl)-4,6-dihydropyrrolo[3,4-c]pyrazole (I, Rₐ═R_{b}═R_{c}═R_{d}═H, R═H, R₁═t-Butyloxycarbonyl(BOC), R₂=Trimethylsilanyl-ethoxymethyl (SEM)).

A solution of 5-tert-butyloxycarbonyl-1(2)H-4,6-dihydropyrrolo[3,4-c]pyrazole (0.7 g, 3.35 mmol) in dry tetrahydrofurane (3 ml) was added dropwise to a suspension of 60% sodium hydride (0.147 g, 3.68 mmol) in dry tetrahydrofurane (2 ml), maintained at room temperature under an Argon atmosphere. After 1 hour, the mixture was cooled to 0° C. and added with a solution of trimethylsilylethyloxymethyl chloride (SEMCl, 0.651 ml, 3.68 mmol) in dry tetrahydrofurane (2 ml). The reaction mixture was then allowed to warm to room temperature and stirring was continued for about 20 hours. After addition of water (10 ml), the mixture was extracted with ethyl acetate (15 ml×4). The organic layers were combined, dried over sodium sulphate, filtered and evaporated to dryness under vacuum. The crude material was purified by flash chromatography on silica gel, using cyclohexane:ethyl acetate 80:20 as eluent to yield the title compound (yellow oil, 0.85 g, 75% yield) as a mixture of 1-SEM and 2-SEM regioisomers (30:70), which were used without being separated.

¹H-NM (DMSO-d₆) δ ppm: 7.7(s,1H); 7.32(s,1H); 5.34(s,1H); 5.33(s,1H); 4.4(m, 4H); 4.29(m, 4H); 3.48(m,2×2H); 1.42(s,2×9H); 0.81(m,2×2H); −0.06(m, 2×9H).

### EXAMPLE 4

Preparation of 3-boronic acid-5-tert-butyloxycarbonyl-1-(2-Trimethylsilanyl-ethoxymethyl)-4,6-dihydropyrrolo[3,4-c]pyrazole and 3-boronic acid-5-tert-butyloxycarbonyl-2-(2-Trimethylsilanyl-ethoxymethyl)-4,6-dihydropyrrolo[3,4-c]pyrazole (I, Rₐ═R_{b}═R_{c}═R_{d}═H, R═B(OH)₂, R₁=t-Butyloxycarbonyl(BOC), R₂=Trimethylsilanyl-ethoxymethyl (SEM)).

n-Buthyllithium (1.6M in n-hexane, 0.75 ml, 1.2 mmol) was slowly added to a solution of the mixture of 5-tert-butyloxycarbonyl-1-(and 2)-(2-Trimethylsilanyl-ethoxymethyl)-4,6-dihydropyrrolo[3,4-c]pyrazole regioisomers (0.339 g, 1 mmol) in dry tetrahydrofurane (4 ml), maintained under stirring at −7° C., under an argon atmosphere.

After 30 minutes, triisopropyl borate (1.15 ml, 5 mmol) was added dropwise, while keeping the temperature at −78° C. The reaction mixture was allowed to spontaneously warm to room temperature and stirring was continued for about 4.5 hours before quenching with 2N HCl to pH6; water (5 ml) was added and the mixture was extracted 15 with ethyl acetate (15 ml×4). The organic layers were combined, washed with brine, dried over sodium sulphate, filtered and dried under vacuum to yield the title compound (light orange oil which solidifies on standing, 350 mg) as a mixture of 1-SEM and 2-SEM regioisomers, which was used without any further purification.

¹H-NMR (DMSO-d₆) δ ppm: 8.3(m,2H); 7.65(m,2H); 5.54(s,1H); 5.34(s,1H); 4.4-4.3(m, 2×4H); 3.6-3.4(m,2×2H); 1.43(s,2×9H); 0.6(m,2×2H); −0.06-−0.07(m, 2×9H).

### EXAMPLE 5

Preparation of 5-tert-butyloxycarbonyl-3-phenyl-1-(2-trimethylsilanyl-ethoxymethyl)-4,6-dihydropyrrolo[3,4-c]pyrazole (I, Rₐ═R_{b}═R_{c}═R_{d}═H, R=Ph, R₁=t-Butyloxycarbonyl (BOC), R₂=Trimethylsilanyl-ethoxymethyl (SEM)) .

A mixture of 3-boronic acid-5-tert-butyloxycarbonyl-1-(2-Trimethylsilanyl-ethoxymethyl)-4,6-dihydropyrrolo[3,4-c]pyrazole (70%, 0.060 g, 0.16 mmol), iodobenzene (0.005 ml, 0.044 mmol), sodium carbonate (0.055 g, 0.52 mmol) and palladium(0)tetrakis (2 mg, 5%) in water (0.16 ml)-Dimethoxyethane (1 ml) was heated under an Argon atmosphere at 80° C. for about 6 hours. The mixture was diluted with ethyl acetate (5 ml), washed with water (3 ml), brine (3 ml), dried over sodium sulphate, filtered and evaporated to dryness. The crude material was purified by flash chromatography to yield the title compound as a light yellow solid (20 mg).

### EXAMPLE 6

Preparation of 1-ethoxycarbonyl-5-(3-methylbutanoyl)-3-iodo-4,6-dihydropyrrolo[3,4-c]pyrazole (I, Rₐ═R_{b}═R_{c}═R_{d}═H, R=Iodo, R₁=3-methylbutanoyl, R₂=1-ethoxycarbonyl).

A solution of 5-tert-butyloxycarbonyl-1-ethoxycarbonyl-3-iodo-4,6-dihydropyrrolo[3,4-c]pyrazole (0.7 g, 1.72 mmol) in dichloromethane (40 ml) was treated with trifluoroacetic acid (9 ml) at room temperature for about 4 hours. After removal of the solvents, the crude salt was dissolved with dry tetrahydrofurane (40 ml) and added with diisopropyl ethyl amine (1.47 ml, 8.6 mmol) and isovaleroyl chloride (0.23 ml, 1.89 ml) diluted with dry tetrahydrofurane (2 ml). The reaction mixture was stirred at room temperature for about 20 hours; the solvent was evaporated under vacuum and the crude material was dissolved with dichloromethane (25 ml), washed with water (15 ml), brine (15 ml), dried over sodium sulphate, filtered and dried under vacuum to yield the title compound as a light brown solid which was used without any further purification (0.65 g, yield 96%).

¹H-NMR (DMSO-d₆) δ ppm: 4.5(m, 2H); .4.38(m, 2H); 4.25(m,2H); 2.18(m,2H) 1.32(m,3H); 0.92(m,6H).

Operating in an analogous way, the following compounds are also obtained:

1-ethoxycarbonyl-3-iodo-5-isopropylaminocarbonyl-4,6-dihydropyrrolo[3,4-c]pyrazole ¹H-NMR (DMSO-d₆) δ ppm: 6.07(m,1H); 4.59(m, 2H); 4.38(m, 2H); 4.21(m,2H); 3.78(m,1H); 1.32(m,3H); 1.08(m,6H).

### EXAMPLE 7

Preparation of 5-isopropylaminocarbonyl-3-(pyrrol-2-yl)-4,6-dihydropyrrolo[3,4-c]pyrazole (I, Rₐ═R_{b}═R_{c}═R_{d}═H, R=pyrrol-2-yl, R₁=3-isopropylaminocarbonyl, R₂═H).

A mixture of 3-iodo-5-isopropylaminocarbonyl-4,6-dihydropyrrolo[3,4-c]pyrazole (0.15 g, 0.38 mmol), 1-tert-butyloxycarbonyl-pyrrole-2-boronic acid (0.191 g, 0.95 mmol), 2M potassium phosphate in water (1 ml) and palladium(0)tetrakis (22 mg, 5%) in Dimethoxyethane (4 ml) was heated under an Argon atmosphere at 80° C. for about 7 hours. The mixture was diluted with ethyl acetate (8 ml), washed with water (5 ml), brine (5 ml), dried over sodium sulphate, filtered and evaporated to dryness. The crude material was purified by flash chromatography, using dichloromethane:methanol 95:5 as eluent to yield the title compound as a light yellow solid (17 mg). ¹H-NMR (DMSO-d₆) δ ppm: 6.82-6.10(m,3H); 5.86(d,1H); 4.42(m, 4H); 3.79(m,1H); 1.10(m,6H).

Operating in an analogous way, the following compounds were also obtained: using 2M caesium carbonate as a base:

5-tert-butyloxycarbonyl-3-(1-tert-butyloxycarbonyl-pyrrol-2-yl)-4,6-dihydropyrrolo[3,4-c]pyrazole (I, Rₐ═R_{b}═R_{c}═R_{d}═H, R=1-tert-butyloxycarbonyl-pyrrol-2-yl, R₁=tert-butyloxycarbonyl R₂═H).

Using sodium carbonate as a base:

5-tert-butyloxycarbonyl-3-(1-tert-butyloxycarbonyl-indol-2-yl)-4,6-dihydropyrrolo[3,4-c]pyrazole (I, Rₐ═R_{b}═R_{c}═R_{d}═H, R=1-tert-butyloxycarbonyl-indol-2-yl, R₁=tert-butyloxycarbonyl, R₂═H);

3-(1-tert-butyloxycarbonyl-indol-2-yl)-5(3-methylbutanoyl)-4,6-dihydropyrrolo[3,4-c]pyrazole (I, Rₐ═R_{b}═R_{c}═R_{d}═H, R=1-tert-butyloxycarbonyl-indol-2-yl, R₁=3-methylbutanoyl, R₂ H).

¹H-NMR (DMSO-d₆) δ ppm: 12.94(s,1H); 7.47(m,4H); 6.91(s,1H); 4.61(m, 4H); 2.18(m,2H); 2.05(m,1H); 1.42(s,9H); 0.91(m,6H).

Using potassium carbonate as a base and a mixture of toluene:ethanol:water 2:1:1 as solvent:

5-tert-butyloxycarbonyl-3-(4-methoxyphenyl)-4,6-dihydropyrrolo[3,4-c]pyrazole (I, Rₐ═R_{b}═R_{c}═R_{d}═H, R=4-methoxyphenyl, R₁=t-buthoxycarbonyl, R₂═H).

¹H NMR (CDCl₃) δ ppm: 7.4-7.31 (2H, m), 6.95-6.89 (2H, m), 4.50-4.31 (4H, m), 3.78 (3br. s), 1.48 (9H, br. s)

### EXAMPLE 8

Preparation of 3-(indol-2-yl)-5-(3-methylbutanoyl)-4,6-dihydropyrrolo[3,4-c]pyrazole (I, Rₐ═R_{b}═R_{c}═R_{d}═H, R=indol-2-yl, R₁=3-methylbutanoyl, R₂═H).

A solution of 3-(1-tert-butyloxycarbonyl-indol-2-yl)-5-(3-methylbutanoyl)-4,6-dihydropyrrolo[3,4-c]pyrazole (0.2 g, 0.49 mmol) in dichloromethane (3.5 ml) was treated with trifluoroacetic acid (0.74 ml), at room temperature for about 24 hours. After removal of the solvents under vacuum, the mixture was diluted with dichloromethane (15 ml), washed with saturated sodium bicarbonate, dried over sodium sulphate, filtered and evaporated to dryness. The crude material was purified by flash chromatography, using dichloromethane:methanol 95:5, 90:10 to yield the title compound as beige solid (0.1 g, 65%).

¹H-NMR (DMSO-d₆) δ ppm: 13.05(s,1H); 11.22 (bs,1H); 7.47(m,2H); 6.99(m,2H); 6.72(bs,1H); 4.80(m, 4H); 2.27(m,2H); 2.1 1(m,1H); 0.95(m,6H).

Operating in an analogous way, the following compound was also obtained

3-(1-H-indol-2-yl)-4,6-dihydropyrrolo[3,4-c]pyrazole (I, Rₐ═R_{b}═R_{c}═R_{d}═H, R=indol-2-yl, R₁═H, R₂═H).

¹H-NMR (DMSO-d₆) δ ppm: 12.71(bs,1H); 11.08 (bs,1H); 6.97(m,2H); 6.72 (s,1H); 6.60(bs,1H); 6.72(bs,1H); 4.07-3.89(m, 4H).

### EXAMPLE 9

Preparation of 5-tert-butyloxycarbonyl-1-ethoxycarbonyl-3-iodo-4,6-dihydropyrrolo[3,4-c]pyrazole (I, Rₐ═R_{b}═R_{c}═R_{d}═H, R=Iodo, R₁=t-Butyloxycarbonyl(BOC), R₂=ethoxycarbonyl).

Isoamyl nitrite (18.2 ml, 135.2 mmol) was slowly added to a mixture of Iodine (20.58 g, 81.11 mmol) in 145 mL of anhydrous dichloromethane, at +22° C. To this dark mixture a solution of 5-tert-butyloxycarbonyl-1-ethoxycarbonyl-3-amino-4,6-dihydropyrrolo[3,4-c]pyrazole (20.03 g, 67.6 mmol) in 140 mL of dichloromethane was added dropwise over 100 min at +22° C. The internal temperature rose to +28° C. and gas evolved during the addition. After 1 hour stirring at room temperature, the reaction mixture was slowly poured in 800 ml of 10% sodium metabisulfite. The phases were separated and the aqueous was extracted twice with 300 mL dichloromethane. The combined extracts were dried over anhydrous sodium sulfate and the solvent evaporated 25 under vacuum. This raw material was purified by flash chromatography eluting with 20:80 EtOAc/cyclohexane. A light yellow product (25.5 g) was obtained which was finally purified with MTBE (60 mL) and n-hexane (60 mL): 21.8 g of high purity, white product was isolated (79% yield). m.p. 166-168° C.

¹H-NM(DMSO-d₆) δ ppm: 4.58(m, 2H); 4.38(q,2H); 4.24(m, 2H); 1.43(s,9H); 1.32(t,3H).

### EXAMPLE 10

Preparation of 5-tert-butyloxycarbonyl-3-iodo-4,6-dihydropyrrolo[3,4-c]pyrazole (I, Rₐ═R_{b}═R_{c}═R_{d}═H. R=Iodo, R₁=t-Butyloxycarbonyl(BOC), R₂═H). 1-ethoxycarbonyl-3-iodo-5-tert-butyloxycarbonyl-4,6-dihydropyrrolo[3,4-c]pyrazole (270 mg, 0.66 mmol) was stirred with a mixture of MeOH (2 ml) and triethylamine (0.5 ml) at room temperature for about 30 min.

The solvents were evaporated and the compound was dried under vacuum. White solid (220 mg).

### EXAMPLE 11

Preparation of 5-tert-butyloxycarbonyl-3-phenyl-4,6-dihydropyrrolo[3,4-c]pyrazole (I, Rₐ═R_{b}═R_{c}═R_{d}═H, R=Phenyl, R₁=t-Butyloxycarbonyl(BOC), R₂═H).

A mixture of 5-tert-butyloxycarbonyl-1-ethoxycarbonyl-3-iodo-4,6-dihydropyrrolo[3,4-c]pyrazole (60 mg, 0.15 mmol), phenylboronic acid (22 mg, 0.18 mmol), potassium carbonate (31 mg, 0.22 mmol), triethylamine (ml 0.03, 0.22 mmol) and palladiumdichloride-diphenylphosphine (8 mg, 7%) in dioxan/water 10/1 (2 ml) was heated under Argon atmosphere at 80° C. for about 3 hours. The mixture was diluted with ethyl acetate (8 ml), washed with water (5 ml), brine (5 ml), dried over sodium sulphate, filtered and evaporated to dryness. The crude material was purified by flash chromatography, using Ethylacetate/hexane as eluent to yield the title compound as a light yellow solid (27 mg 63%).

### EXAMPLE 12

Preparation of 5-acetyl-3-phenyl-4,6-dihydropyrrolo[3,4-c]pyrazole (I, Rₐ═R_{b}═R_{c}═R_{d}═H, R=Phenyl, R₁=Acetyl, R₂═H).

A solution of 5-tert-butyloxycarbonyl-3-phenyl-4,6-dihydropyrrolo[3,4-c]pyrazole (90 mg, 0.31 mmol) in dichloromethane (3.5 ml) was treated with trifluoroacetic acid (0.5 ml), at room temperature for about 4 hours. After removal of the solvents, the crude salt was dissolved with dry dichloromethane (5 ml) and diisopropylethylamine (0.32 ml, 1.86 mmol) and acetyl chloride (0.07 ml, 0.9 mmol) were added. The reaction mixture was stirred at room temperature for about 2 hours; the crude material was diluted with dichloromethane (25 ml), washed with water (15 ml), brine (15 ml), dried over sodium sulphate, filtered and dried under vacuum. The crude was suspended in a solution of sodium bicarbonate and stirred at room temperature for about 3 hours, then extracted with ethylacetate to yield the title compound as a light brown solid (40 mg).

### EXAMPLE 13

Preparation of 5-tert-butyloxycarbonyl-3-iodo-1-polystyrenemethylaminocarbonyl-4,6-dihydropyrrolo[3,4-c]pyrazole (III, Rₐ═R_{b}═R_{c}═R_{d}═H, R=Iodo, R₁=t-Butyloxycarbonyl(BOC), Q=polystyrenemethylaminocarbonyl).

The isocyanate methylpolystyrene resin (1.14 g, 1,71 mmol) was swelled with 15 ml of dichloromethane, and a solution of 5-tert-butyloxycarbonyl-3-iodo-4,6-dihydropyrrolo[3,4-c]pyrazole (410 mg, 1.22 mmol) in 3 ml of dimethylformamide was added.

The mixture was stirred at room temperature for about 24 hours; after filtration; the resin was washed with dichloromethane (2×20 ml), MeOH (2×20 ml), dimethylformamide (2×20 ml) and dichloromethane (3×20 ml).

The resin was dried under vacuum.

Operating in an analogous way, the following compound was also obtained 5-tert-butyloxycarbonyl-3-(4methoxyphenyl)-1-polystyrenemethylaminocarbonyl-4,6-dihydropyrrolo[3,4-c]pyrazole (III Rₐ═R_{b}═R_{c}═R_{d}═H, R=4-methoxyphenyl, R₁=t-Butyloxycarbonyl(BOC), Q=polystyrenemethylaminocarbonyl).

### EXAMPLE 14

Preparation of 5-tert-butyloxycarbonyl-3-phenyl-1-polystyrenemethylaminocarbonyl-4,6-dihydropyrrolo[3,4-c]pyrazole (III, Rₐ═R_{b}═R_{c}═R_{d}═H, R=Phenyl, R₁=t-Butyloxycarbonyl(BOC), Q=polystyrenemethylaminocarbonyl).

To a suspension of 5-tert-butyloxycarbonyl-3-iodo-1-polystyrenemethylaminocarbonyl-4,6-dihydropyrrolo[3,4-c]pyrazole (117 mg, 0.17 mmol) in dioxan/water 10/1 (3 ml), phenylboronic acid (108 mg, 0.88 mmol), potassium carbonate (171 mg, 0.8 mmol), triethylamine (0.18 ml, 0.8 mmol) and palladiumdichloride diphenylphosphine (25 mg, 20%) were added.

The mixture was stirred at 80° C. for about 8 hours; after filtration, the resin was washed with dichloromethane (2×20 ml), MeoH (2×20 ml), dimethylformamide (2×20 ml) and dichloromethane (3×20 ml).

The resin was dried under vacuum.

Operating in an analogous way, using a suitable boronic acid, the following compounds were also obtained:

5-tert-butyloxycarbonyl-3-(4-phenoxy-phenyl)-1-polystyrenemethylaminocarbonyl-4,6-dihydropyrrolo[3,4-c]pyrazole (III, Rₐ═R_{b}═R_{c}═R_{d}═H, R=4-phenoxy-phenyl, R₁=t-Butyloxycarbonyl(BOC), Q=polystyrenemethylaminocarbonyl);

3-(4benzyloxy-phenyl)-5-tert-butyloxycarbonyl-1-polystyrenemethylaminocarbonyl-4,6-dihydropyrrolo[3,4-c]pyrazole (m, Rₐ═R_{b}═R_{c}═R_{d}═H, R=4-benzyloxy-phenyl, R₁=t-Butyloxycarbonyl(BOC), Q=polystyrenemethylaminocarbonyl);

5-tert-butyloxycarbonyl-3-(5-chloro-thiophen-2-yl)-1-polystyrenemethylaminocarbonyl-4,6-dihydropyrrolo[3,4-c]pyrazole (III, Rₐ═R_{b}═R_{c}═R_{d}═H, R=5-chloro-thiophen-2-yl, R₁-t-Butyloxycarbonyl(BOC), Q=polystyrenemethylaminocarbonyl);

5-tert-butyloxycarbonyl-3-(4-methoxy-phenyl)1-polystyrenemethylaminocarbonyl-4,6-dihydropyrrolo[3,4-c]pyrazole (III, Rₐ═R_{b}═R_{c}═R_{d}═H, R=4-methoxy-phenyl, R₁=t-Butyloxycarbonyl(BOC), Q=polystyrenemethylaminocarbonyl) and

5-tert-butyloxycarbonyl-3-(4-dimethylamino-phenyl)-1-polystyrenemethylaminocarbonyl-4,6-dihydropyrrolo[3,4-c]pyrazole (III, Rₐ═R_{b}═R_{c}═R_{d}═H, R=4-dimethylamino-phenyl, R₁=t-Butyloxycarbonyl(BOC), Q=polystyrenemethylaminocarbonyl).

### EXAMPLE 15

Preparation of 5-tert-butyloxycarbonyl-3-phenylethynyl-1-polystyrenemethylaminocarbonyl-4,6-dihydropyrrolo[3,4-c]pyrazole (I, Rₐ═R_{b}═R_{c}═R_{d}═H, R=Phenylethynyl, R₁=t-Butyloxycarbonyl(BOC), Q=polystyrenemethylaminocarbonyl).

To a suspension of 5-tert-butyloxycarbonyl-3-iodo-1-polystyrenemethylaminocarbonyl-4,6-dihydropyrrolo[3,4-c]pyrazole (200 mg, 0.21 mmol) in dioxan (2 ml), phenylethyne (0.23 ml, 2 mmol), CuI (20 mg, 50%), triethylamine (0.12 ml, 1.5 mmol) and palladiumdichloride diphenylphosphine (29 mg, 20%) were added.

The mixture was stirred at 80° C. for about 8 hours; after filtration, the resin was washed with dichlorometane (2×20 ml), MeOH (2×20 ml), dimethylformamide (2×20 ml) and with dichloromethane (3×20 ml).

The resin was dried under vacuum.

### EXAMPLE 16

Preparation of 3-phenyl-1-polystyrenemethylaminocarbonyl-4,6-dihydropyrrolo[3,4-c]pyrazole (III, Rₐ═R_{b}═R_{c}═R_{d}═H, R=Phenyl, R₁═H, Q=polystyrenemethylaminocarbonyl).

To 5-tert-butyloxycarbonyl-3-phenyl-1-polystyrenemethylaminocarbonyl-4,6-dihydropyrrolo[3,4-c]pyrazole swelled in dichloromethane (5 ml) trifluoroacetic acid (1 ml) was added.

The mixture was stirred at room temperature for about 4 hours, after filtration, the resin was washed with dichlorometane (2×20 ml), MeOH (2×20 ml), dimethylformamide (2×20 ml) and dichloromethane (3×20 ml).

The resin was dried under vacuum.

Operating in an analogous way, the following compounds were also obtained:

3-(4-phenoxy-phenyl)-1-polystyrenemethylaminocarbonyl-4,6-dihydropyrrolo[3,4-c]pyrazole (III, Rₐ═R_{b}═R_{c}═R_{d}═H, R=Phenyl, R₁═H, Q=polystyrenemethylaminocarbonyl);

3-(4-benzyloxy-phenyl)-1-polystyrenemethylaminocarbonyl-4,6-dihydropyrrolo[3,4-c]pyrazole (III, Rₐ═R_{b}═R_{c}═R_{d}═H, R=4-Benzyloxyphenyl, R₁═H, Q=polystyrenemethylaminocarbonyl);

3-(5-chloro-thiophen-2-yl)-1-polystyrenemethylaminocarbonyl-4,6-dihydro-pyrrolo[3,4-c]pyrazole (III, Rₐ═R_{b}═R_{c}═R_{d}═H, R=5-Chloro-thiophen-2-yl, R₁═H, Q=polystyrenemethylaminocarbonyl);

3-(4-methoxy-phenyl)-1-polystyrenemethylaminocarbonyl-4,6-dihydropyrrolo[3,4-c]pyrazole (III, Rₐ═R_{b}═R_{c}═R_{d}═H, R=4-Methoxyphenyl, R₁═H, Q=polystyrenemethylaminocarbonyl);

3-(4dimethylamino-phenyl)-1-polystyrenemethylaminocarbonyl-4,6-dihydropyrrolo[3,4-c]pyrazole (III, Rₐ═R_{b}═R_{c}═R_{d}═H, R=4-Dimethylaminophenyl, R₁═H, Q=polystyrenemethylaminocarbonyl);

3-phenylethynyl-1-polystyrenemethylaminocarbonyl-4,6-dihydropyrrolo[3,4-c]pyrazole (III, Rₐ═R_{b}═R_{c}═R_{d}═H, R=Phenylethynyl, R₁═H, Q=polystyrenemethylaminocarbonyl) and

3-(4-methoxyphenyl)-1-polystyrenemethylaminocarbonyl-4,6-dihydropyrrolo[3,4-c]pyrazole (III, Rₐ═R_{b}═R_{c}═R_{d}═H, R=4-methoxyphenyl, R₁═H, Q=polystyrenemethylaminocarbonyl).

### EXAMPLE 17

Preparation of 5-acetyl-3-phenyl-1-polystyrenemethylaminocarbonyl-4,6-dihydropyrrolo[3,4-c]pyrazole (III, Rₐ═R_{b}═R_{c}═R_{d}═H, R=Phenyl, R₁=Acetyl, Q=polystyrenemethylaminocarbonyl).

To 3-phenyl-1-polystyrenemethylaminocarbonyl-4,6-dihydropyrrolo[3,4-c]pyrazole swelled in dichloromethane (5 ml) diisopropylethylamine (0.21 ml, 1.24 mmol) and acetylchloride (0.06 ml. 0.88 mmol) were added.

The mixture was stirred at room temperature for about 24 hours; after filtration, the resin was washed with dichlorometane (2×20 ml), MeOH (2×20 ml), dimethylformamide (2×20 ml) and dichloromethane (3×20 ml). The resin was dried under vacuum.

Operating in an analogous way, the following compounds were also obtained:

5-acetyl-3-(4-phenoxy-phenyl)-1-polystyrenemethylaminocarbonyl-4,6-dihydropyrrolo[3,4-c]pyrazole (III, Rₐ═R_{b}═R_{c}═R_{d}═H, R=4-Phenoxyphenyl, R₁=Acetyl, Q=polystyrenemethylaminocarbonyl);

5-acetyl-3-(4-benzyloxy-phenyl)-1-polystyrenemethylaminocarbonyl-4,6-dihydropyrrolo[3,4-c]pyrazole (III, Rₐ═R_{b}═R_{c}═R_{d}═H, R=4-Benzyloxyphenyl, R₁=Acetyl, Q=polystyrenemethylaminocarbonyl);

5-acetyl-3-(5-chloro-thiophen-2-yl)-1-polystyrenemethylaminocarbonyl-4,6-dihydropyrrolo[3,4-c]pyrazole (III, Rₐ═R_{b}═R_{c}═R_{d}═H, R=5-Chloro-thiophen-2-yl, R₁=Acetyl, Q=polystyrenemethylaminocarbonyl);

5-acetyl-3-(4-methoxy-phenyl)-1-polystyrenemethylaminocarbonyl-4,6-dihydropyrrolo[3,4-c]pyrazole (III Rₐ═R_{b}═R_{c}═R_{d}═H, R=4-Methoxyoxyphenyl, R₁=Acetyl, Q=polystyrenemethylaminocarbonyl);

5-acetyl-3-(4-dimethylamino-phenyl)-1-polystyrenemethylaminocarbonyl-4,6-dihydropyrrolo[3,4-c]pyrazole (III, Rₐ═R_{b}═R_{c}═R_{d}═H, R=4-Dimethylamino-phenyl R₁=Acetyl, Q=polystyrenemethylaminocarbonyl);

5-acetyl-3-phenylethynyl-1-polystyrenemethylaminocarbonyl-4,6-dihydropyrrolo[3,4-c]pyrazole (III, Rₐ═R_{b}═R_{c}═R_{d}═H, R=Phenylethynyl, R₁=Acetyl Q=polystyrenemethylaminocarbonyl) and

3-(4-t-butylphenyl-5-(2-phenoxypropionyl)-1-polystyrenemethylaminocarbonyl-4,6-dihydropyrrolo[3,4-c]pyrazole (III, Rₐ═R_{b}═R_{c}═R_{d}═H=4-t-butylyphenyl, R₁-2-phenoxypropionyl, Q=polystyrenemethylaminocarbonyl).

### EXAMPLE 18

Preparation of 5-isopropylaminocarbonyl-3-phenyl-1-polystyrenemethylaminocarbonyl-4,6-dihydropyrrolo[3,4-c]pyrazole (III, Rₐ═R_{b}═R_{c}═R_{d}═H, R=Phenyl, R₁=Isopropylaminocarbonyl, Q=polystyrenemethylaminocarbonyl).

To 3-phenyl-1-polystyrenemethylaminocarbonyl-4,6-dihydropyrrolo[3,4-c]pyrazole swelled in dichloromethane (5 ml) isopropylisocyanate (0.09 ml. 0.88 mmol) was added. The mixture was stirred at room temperature for about 24 hours; after filtration, the resin was washed with dichloromethane (2×20 ml), MeOH (2×20 ml), dimethylformamide (2×20 ml) and dichloromethane (3×20 ml). The resin was dried under vacuum.

Operating in an analogous way, the following compounds were also obtained:

5-isopropylaminocarbonyl-3-(4-phenoxy-phenyl)-1-polystyrenemethylaminocarbonyl-4,6-dihydropyrrolo[3,4-c]pyrazole (III, Rₐ═R_{b}═R_{c}═R_{d}═H, R=4-Phenoxyphenyl, R₁=Isopropylaminocarbonyl, Q=polystyrenemethylaminocarbonyl);

3-(4-benzyloxy-phenyl)-5-isopropylaminocarbonyl-1-polystyrenemethylaminocarbonyl-4,6-dihydropyrrolo[3,4-c]pyrazole (III, Rₐ═R_{b}═R_{c}═R_{d}═H, R=4-Benzyloxyphenyl, R₁=Isopropylaminocarbonyl, Q=polystyrenemethylaminocarbonyl);

3-(5-chloro-thiophen-2-yl)-5-isopropylaminocarbonyl-1-polystyrenemethylaminocarbonyl-4,6-dihydropyrrolo[3,4-c]pyrazole (III, Rₐ═R_{b}═R_{c}═R_{d}═H, R=5-Chloro-thiophen-2-yl, R₁=Isopropylaminocarbonyl, Q=polystyrenemethylaminocarbonyl);

5-isopropylaminocarbonyl-3-(4-methoxy-phenyl)-1-polystyrenemethylamino carbonyl-4,6-dihydro-pyrrolo[3,4-c]pyrazole (III, Rₐ═R_{b}═R_{c}═R_{d}═H, R=4Methoxy-phenyl, R₁=Isopropylaminocarbonyl, Q=polystyrenemethylaminocarbonyl);

3-(4-dimethylamino-phenyl)-5-isopropylaminocarbonyl-1-polystyrenemethylaminocarbonyl-4,6-dihydropyrrolo[3,4-c]pyrazole (III, Rₐ═R_{b}═R_{c}═R_{d}═H, R=Dimethylamino-phenyl, R₁=Isopropylaminocarbonyl, Q=polystyrenemethylaminocarbonyl);

5-isopropylaminocarbonyl-3-phenylethynyl-1-polystyrenemethylaminocarbonyl-4,6-dihydropyrrolo[3,4-c]pyrazole (III, Rₐ═R_{b}═R_{c}═R_{d}═H, R=Phenylethynyl, R₁=Isopropylaminocarbonyl, Q=polystyrenemethylaminocarbonyl) and

3-(2,5-dimethylphenyl)-5-n-propylaminocarbonyl-1-polystyrenemethylaminocarbonyl-4,6-dihydropyrrolo[3,4-c]pyrazole (III, Rₐ═R_{b}═R_{c}═R_{d}═H, R=4-(2,5-dimethylphenyl), R₁=n-propylaminocarbonyl, Q=polystyrenemethylaminocarbonyl).

### EXAMPLE 19

Preparation of 5-acetyl-3-phenyl-4,6-dihydropyrrolo[3,4-c]pyrazole (Rₐ═R_{b}═R_{c}═R_{d}═H, R=Phenyl, R₁=Acetyl, R₂═H).

To 5-acetyl-3-phenyl-1-polystyrenemethylaminocarbonyl-4,6-dihydropyrrolo[3,4-c]pyrazole (200 mg) swelled in dioxan (3 ml), sodium hydroxide (35% in water) was added (0.4 ml) and the mixture was stirred at 40° C. for about 90 hours.

After neutralization of the solution, the mixture was filtered and the desired product was dried under vacuum: a white solid (40 mg) was obtained.

Operating in an analogous way, the following compounds were also obtained.

5-Isopropylaminocarbonyl-3-phenyl-4,6-dihydropyrrolo[3,4-c]pyrazole (Rₐ═R_{b}═R_{c}═R_{d}═H, R=Phenyl, R₁=Isopropylaminocarbonyl, R₂═H).

¹H-NMR (DMSO-d₆) δ ppm: 13.12 (s,1H); 7.58-7.32(m,5H); 5.97(d,1H); 4.53(m, 4H); 3.38(m,1H); 1.10(m,6H);

5Acetyl-3-(4-phenoxy-phenyl)-4,6-dihydropyrrolo[3,4-c]pyrazole (Rₐ═R_{b}═R_{c}═R_{d}═H, R=4-Phenoxy-phenyl, R₁=Acetyl, R₂═H).

¹H-NMR (DMSO-d₆) δ ppm: 13.11(s,1H); 7.62-7.05(m,9H); 4.78(m, 4H); 2.06(s,3H)

5-Isopropylaminocarbonyl-3-(4-phenoxy-phenyl)-4,6-dihydropyrrolo[3,4-c]pyrazole (Rₐ═R_{b}═R_{c}═R_{d}═H, R=Phenoxy-phenyl, R₁=Isopropylaminocarbonyl, R₂═H).

¹H-NMR (DMSO-d₆) δ ppm: 13.06 (s,1H); 7.59-7.04(m,9H); 5.93(d,1H); 4.51-4.42(m, 4H); 3.80(m,1H); 1.09(m,6H).

5-Acetyl-3-(4-benzyloxy-phenyl)-4,6-dihydropyrrolo[3,4-c]pyrazole (Rₐ═R_{b}═R_{c}═R_{d}═H, R=4-Benzyloxy-phenyl, R₁=Acetyl, R₂═H):

3-(4-benzyloxy-phenyl)-5-isopropylaminocarbonyl-4,6-dihydropyrrolo[3,4-c]pyrazole (Rₐ═R_{b}═R_{c}═R_{d}═H, R=4-Benzyloxy-phenyl , R₁=Isopropylaminocarbonyl, R₂═H).

5-Acetyl-3-(5-chloro-thiophen-2-yl)-4,6-dihydropyrrolo[3,4-c]pyrazole (Rₐ═R_{b}═R_{c}═R_{d}═H, R=5-Chloro-thiophen-2-yl, R₁=Acetyl, R₂═H).

¹H-NMR (DMSO-d₆) δ ppm: 13.07(s,1H); 7.14(m,2H); 4.69(m, 4H); 2.04(s,3H).

3-(5-Chloro-thiophen-2-yl)-5-isopropylaminocarbonyl-4,6-dihydropyrrolo[3,4-c]pyrazole (Rₐ═R_{b}═R_{c}═R_{d}═H, R=5-Chloro-thiophen-2-yl, R₁=Isopropylaminocarbonyl, R₂═H).

¹H-NMR (DMSO-d₆) δ ppm: 13.13(s,1H); 7.14(m,2H); 5.94(d,1H); 4.41(m, 4H); 3.79(m,1H); 1.10(m,6H).

5-Acetyl-3-(4-methoxy-phenyl)-4,6-dihydropyrrolo[3,4-c]pyrazole (Rₐ═R_{b}═R_{c}═R_{d}═H, R=4-Methoxy-phenyl, R₁=Acetyl, R₂═H);

5-isopropylaminocarbonyl-3-(4-methoxy-phenyl)-4,6-dihydropyrrolo[3,4-c]pyrazole (Rₐ═R_{b}═R_{c}50 R_{d}═H, R=4-Methoxy-phenyl, R₁=Isopropylaminocarbonyl, R₂═H);

5-acetyl-3-(4-dimethylamino-phenyl)-4,6-dihydropyrrolo[3,4-c]pyrazole (Rₐ═R_{b}═R_{c}═R_{d}═H, R=4-Dimethylamino-phenyl, R₁=Acetyl, R₂═H).

¹H-NMR (DMSO-d₆) δ ppm: 7.44-7.41(dd,2H); 6.75-6.77(d,2H); 4.74-4.21(m, 4H); 2.87(s,6H); 2.00(s,3H).

3-(4-Dimethylamino-phenyl)-5-isopropylaminocarbonyl-4,6-dihydropyrrolo[3,4-c]pyrazole (Rₐ═R_{b}═R_{c}═R_{d}═H, R=4-Dimethylamino-phenyl, R₁=Isopropylaminocarbonyl, R₂═H).

¹H-NMR (DMSO-d₆) δ ppm: 7.40(m,2H); 6.77(m,2H); 4.18(m, 4H); 3.78(m,1H); 2.92 (s,6H); 1.11(m,6H).

5-Acetyl-3-phenylethynyl-4,6-dihydropyrrolo[3,4-c]pyrazole (Rₐ═R_{b}═R_{c}═R_{d}═H, R=Phenylethynyl, R₁=Acetyl, R₂═H).

¹H-NMR (DMSO-d₆) δ ppm: 7.53-7.42(m,5H); 4.35(m, 4H); 3.80(m,1H); 1.03 (m,6H)

5-Isopropylaminocarbonyl-3-phenylethynyl-4,6-dihydropyrrolo[3,4-c]pyrazole (Rₐ═R_{b}═R_{c}═R_{d}═H, R=Phenylethynyl, R₁=Isopropylaminocarbonyl, R₂═H)

3-(2,5-dimethylphenyl)-5-n-propylaminocarbonyl-4,6-dihydropyrrolo[3,4-c]pyrazole (I, Rₐ═R_{b}═R_{c}═R_{d}═H, R=4-(2,5-dimethylphenyl), R₁=n-propylaminocarbonyl, R2=H).

LCMS: m/z 299 [M+H]⁺ @ R_{T} 1:21 min (81% by ELS detection).

3-(4-t-butylphenyl)-5-(2-phenoxypropionyl)-4,6-dihydropyrrolo [3,4-c]pyrazole (I, Rₐ═R_{b}═R_{c}═R_{d}═H, R=t-butylphenyl, R₁=2-phenoxypropionyl, R2=H).

¹H NMR (DMSO-d₆) δ ppm: 7.61-7.53 (2H, m), 7.52-7.45 (2H, m), 7.30-7.22 (2H, m), 6.96-6.87 (3H, m), 5.22-5.12 (1H, m), 4.97-4.84 (1H, m), 4.72-4.62 (2H, m), 4.51-4.47 (1H, m), 1.60-1.50 (3H, m), 1.32 (9H, br. S), pyrazole NH not observed;

LCMS: m/z 390 [M+H]⁺ @ R_{T} 1.57 min (88% by ELS detection).

By proceeding in the same way as described in examples 7, 13, 16, 17, 18 and 19, 1048 products were synthesized in parallel and coded in table III, as formerly indicated; related HPLC retention time together with experimentally found [M+H]+ are reported.

TABLE III

| | | r.t. | |
|---|---|---|---|
| Entry | Compound | (min) | [M + H]+ |
| 1 | A1-M-B1 | 1.24 | 304.1 |
| 2 | A2-M-B1 | 1.26 | 304.1 |
| 3 | A3-M-B1 | 1.1 | 280.1 |
| 4 | A4-M-B1 | 1.22 | 350.1 |
| 5 | A5-M-B1 | 1.24 | 310.1 |
| 6 | A1-M-B2 | 1.3 | 318.2 |
| 7 | A2-M-B2 | 1.33 | 318.2 |
| 8 | A5-M-B2 | 1.31 | 324.1 |
| 9 | A1-M-B3 | 1.38 | 310.2 |
| 10 | A2-M-B3 | 1.4 | 310.2 |
| 11 | A6-M-B3 | 1.29 | 302.1 |
| 12 | A3-M-B3 | 1.24 | 286.1 |
| 13 | A4-M-B3 | 1.35 | 356.2 |
| 14 | A5-M-B3 | 1.38 | 316.1 |
| 15 | A1-M-B4 | 1.02 | 242.1 |
| 16 | A2-M-B4 | 1.06 | 242.1 |
| 17 | A7-M-B4 | 0.98 | 258.1 |
| 18 | A3-M-B4 | 0.88 | 218.1 |
| 19 | A1-M-B5 | 1.5 | 324.2 |
| 20 | A8-M-B5 | 1.48 | 370.2 |
| 21 | A3-M-B5 | 1.37 | 300.2 |
| 22 | A5-M-B5 | 1.52 | 330.2 |
| 23 | A1-M-B6 | 1.35 | 338.1 |
| 24 | A2-M-B6 | 1.37 | 338.1 |
| 25 | A6-M-B6 | 1.27 | 330.0 |
| 26 | A8-M-B6 | 1.34 | 384.1 |
| 27 | A3-M-B6 | 1.22 | 314.1 |
| 28 | A5-M-B6 | 1.36 | 344.1 |
| 29 | A1-M-B7 | 1.29 | 348.2 |
| 30 | A9-M-B7 | 1.32 | 348.2 |
| 31 | A2-M-B7 | 1.32 | 348.2 |
| 32 | A3-M-B7 | 1.17 | 324.1 |
| 33 | A4-M-B7 | 1.27 | 394.2 |
| 34 | A1-M-B8 | 1.24 | 348.1 |
| 35 | A9-M-B8 | 1.26 | 348.1 |
| 36 | A2-M-B8 | 1.26 | 348.1 |
| 37 | A8-M-B8 | 1.22 | 394.1 |
| 38 | A3-M-B8 | 1.1 | 324.1 |
| 39 | A5-M-B8 | 1.24 | 354.1 |
| 40 | A1-M-B9 | 1.31 | 334.1 |
| 41 | A3-M-B9 | 1.2 | 310.1 |
| 42 | A4-M-B9 | 1.3 | 380.2 |
| 43 | A1-M-B10 | 1.36 | 298.2 |
| 44 | A8-M-B10 | 1.34 | 344.2 |
| 45 | A3-M-B10 | 1.23 | 274.1 |
| 46 | A5-M-B10 | 1.37 | 304.1 |
| 47 | A1-M-B11 | 1.27 | 322.1 |
| 48 | A9-M-B11 | 1.3 | 322.1 |
| 49 | A2-M-B11 | 1.3 | 322.1 |
| 50 | A6-M-B11 | 1.2 | 314.1 |
| 51 | A8-M-B11 | 1.27 | 368.1 |
| 52 | A3-M-B11 | 1.15 | 298.1 |
| 53 | A5-M-B11 | 1.28 | 328.1 |
| 54 | A9-M-B12 | 1.27 | 339.1 |
| 55 | A1-M-B13 | 1.24 | 310.1 |
| 56 | A3-M-B13 | 1.11 | 286.1 |
| 57 | A5-M-B13 | 1.25 | 316.1 |
| 58 | A1-M-B14 | 1.18 | 364.2 |
| 59 | A2-M-B14 | 1.21 | 364.2 |
| 60 | A6-M-B14 | 1.11 | 356.1 |
| 61 | A3-M-B14 | 1.06 | 340.1 |
| 62 | A5-M-B14 | 1.18 | 370.1 |
| 63 | A1-M-B15 | 1.14 | 268.1 |
| 64 | A3-M-B15 | 1.01 | 244.1 |
| 65 | A5-M-B15 | 1.17 | 274.1 |
| 66 | A1-M-B16 | 1.25 | 334.1 |
| 67 | A9-M-B16 | 1.28 | 334.1 |
| 68 | A2-M-B16 | 1.28 | 334.1 |
| 69 | A3-M-B16 | 1.13 | 310.1 |
| 70 | A5-M-B16 | 1.25 | 340.1 |
| 71 | A1-M-B17 | 1.2 | 256.1 |
| 72 | A4-M-B17 | 1.12 | 302.1 |
| 73 | A1-M-B18 | 1.33 | 340.1 |
| 74 | A6-M-B18 | 1.26 | 332.1 |
| 75 | A8-M-B18 | 1.32 | 386.1 |
| 76 | A3-M-B18 | 1.21 | 316.1 |
| 77 | A5-M-B18 | 1.33 | 346.1 |
| 78 | A1-M-B19 | 1.25 | 334.1 |
| 79 | A9-M-B19 | 1.27 | 334.1 |
| 80 | A2-M-B19 | 1.27 | 334.1 |
| 81 | A6-M-B19 | 1.17 | 326.1 |
| 82 | A3-M-B19 | 1.12 | 310.1 |
| 83 | A5-M-B19 | 1.25 | 340.1 |
| 84 | A1-M-B20 | 1.14 | 323.1 |
| 85 | A9-M-B20 | 1.18 | 323.1 |
| 86 | A2-M-B20 | 1.17 | 323.1 |
| 87 | A6-M-B20 | 1.07 | 315.1 |
| 88 | A8-M-B20 | 1.14 | 369.1 |
| 89 | A7-M-B20 | 1.1 | 339.1 |
| 90 | A3-M-B20 | 1.01 | 299.1 |
| 91 | A5-M-B20 | 1.15 | 329.1 |
| 92 | A1-M-B21 | 1.27 | 322.1 |
| 93 | A9-M-B21 | 1.29 | 322.1 |
| 94 | A2-M-B21 | 1.29 | 322.1 |
| 95 | A6-M-B21 | 1.19 | 314.1 |
| 96 | A8-M-B21 | 1.25 | 368.1 |
| 97 | A7-M-B21 | 1.21 | 338.1 |
| 98 | A3-M-B21 | 1.14 | 298.1 |
| 99 | A5-M-B21 | 1.3 | 328.1 |
| 100 | A1-M-B22 | 1.32 | 296.2 |
| 101 | A9-M-B22 | 1.38 | 296.2 |
| 102 | A2-M-B22 | 1.35 | 296.2 |
| 103 | A6-M-B22 | 1.23 | 288.1 |
| 104 | A8-M-B22 | 1.31 | 342.2 |
| 105 | A3-M-B22 | 1.18 | 272.1 |
| 106 | A5-M-B22 | 1.32 | 302.1 |
| 107 | A1-M-B23 | 1.36 | 332.2 |
| 108 | A8-M-B23 | 1.35 | 378.2 |
| 109 | A3-M-B23 | 1.25 | 308.1 |
| 110 | A1-M-B24 | 1.34 | 348.2 |
| 111 | A9-M-B24 | 1.37 | 348.2 |
| 112 | A7-M-B24 | 1.29 | 364.2 |
| 113 | A3-M-B24 | 1.22 | 324.1 |
| 114 | A1-M-B25 | 1.32 | 338.1 |
| 115 | A9-M-B25 | 1.33 | 338.1 |
| 116 | A2-M-B25 | 1.33 | 338.1 |
| 117 | A8-M-B25 | 1.29 | 384.1 |
| 118 | A7-M-B25 | 1.25 | 354.1 |
| 119 | A3-M-B25 | 1.18 | 314.1 |
| 120 | A8-M-B26 | 1.22 | 375.1 |
| 121 | A1-M-B27 | 1.24 | 282.2 |
| 122 | A2-M-B27 | 1.28 | 282.2 |
| 123 | A3-M-B27 | 1.11 | 258.1 |
| 124 | A1-M-B28 | 1.32 | 340.1 |
| 125 | A2-M-B28 | 1.37 | 340.1 |
| 126 | A8-M-B28 | 1.31 | 386.1 |
| 127 | A3-M-B28 | 1.2 | 316.1 |
| 128 | A1-M-B29 | 1.04 | 272.1 |
| 129 | A1-M-B30 | 1.21 | 394.2 |
| 130 | A9-M-B30 | 1.24 | 394.2 |
| 131 | A2-M-B30 | 1.24 | 394.2 |
| 132 | A6-M-B30 | 1.24 | 386.1 |
| 133 | A7-M-B30 | 1.17 | 410.2 |
| 134 | A4-M-B30 | 1.21 | 440.2 |
| 135 | A1-M-B31 | 1.31 | 340.1 |
| 136 | A9-M-B31 | 1.33 | 340.1 |
| 137 | A2-M-B31 | 1.33 | 340.1 |
| 138 | A6-M-B31 | 1.23 | 332.1 |
| 139 | A8-M-B31 | 1.29 | 386.1 |
| 140 | A7-M-B31 | 1.26 | 356.1 |
| 141 | A3-M-B31 | 1.18 | 316.1 |
| 142 | A1-M-B32 | 1.28 | 322.1 |
| 143 | A2-M-B32 | 1.3 | 322.1 |
| 144 | A6-M-B32 | 1.21 | 314.1 |
| 145 | A3-M-B32 | 1.16 | 298.1 |
| 146 | A1-M-B33 | 1.3 | 284.2 |
| 147 | A2-M-B33 | 1.33 | 284.2 |
| 148 | A8-M-B33 | 1.29 | 330.2 |
| 149 | A3-M-B33 | 1.17 | 260.1 |
| 150 | A1-M-B34 | 1.51 | 326.2 |
| 151 | A9-M-B34 | 1.54 | 326.2 |
| 152 | A2-M-B34 | 1.53 | 326.2 |
| 153 | A6-M-B34 | 1.42 | 318.2 |
| 154 | A8-M-B34 | 1.48 | 372.2 |
| 155 | A7-M-B34 | 1.44 | 342.2 |
| 156 | A3-M-B34 | 1.38 | 302.2 |
| 157 | A1-M-B35 | 1.33 | 382.0 |
| 158 | A9-M-B35 | 1.34 | 382.0 |
| 159 | A2-M-B35 | 1.34 | 382.0 |
| 160 | A6-M-B35 | 1.24 | 374.0 |
| 161 | A7-M-B35 | 1.26 | 398.0 |
| 162 | A3-M-B35 | 1.19 | 358.0 |
| 163 | A1-M-B36 | 1.28 | 324.1 |
| 164 | A2-M-B36 | 1.31 | 324.1 |
| 165 | A3-M-B36 | 1.16 | 300.1 |
| 166 | A1-M-B37 | 1.44 | 346.2 |
| 167 | A2-M-B37 | 1.47 | 346.2 |
| 168 | A6-M-B37 | 1.51 | 338.1 |
| 169 | A8-M-B37 | 1.43 | 392.2 |
| 170 | A3-M-B37 | 1.32 | 322.1 |
| 171 | A1-M-B38 | 1.52 | 376.2 |
| 172 | A9-M-B38 | 1.55 | 376.2 |
| 173 | A1-M-B39 | 1.29 | 397.2 |
| 174 | A8-M-B39 | 1.28 | 443.2 |
| 175 | A7-M-B39 | 1.25 | 413.2 |
| 176 | A1-M-B40 | 1.28 | 340.1 |
| 177 | A9-M-B40 | 1.3 | 340.1 |
| 178 | A2-M-B40 | 1.3 | 340.1 |
| 179 | A6-M-B40 | 1.2 | 332.1 |
| 180 | A8-M-B40 | 1.27 | 386.1 |
| 181 | A7-M-B40 | 1.23 | 356.1 |
| 182 | A3-M-B40 | 1.15 | 316.1 |
| 183 | A1-M-B41 | 1.38 | 382.0 |
| 184 | A8-M-B41 | 1.37 | 428.1 |
| 185 | A3-M-B41 | 1.25 | 358.0 |
| 186 | A1-M-B42 | 1.32 | 318.2 |
| 187 | A2-M-B42 | 1.34 | 318.2 |
| 188 | A8-M-B42 | 1.31 | 364.2 |
| 189 | A3-M-B42 | 1.19 | 294.1 |
| 190 | A1-M-B43 | 1.21 | 302.1 |
| 191 | A2-M-B43 | 1.24 | 302.1 |
| 192 | A8-M-B43 | 1.21 | 348.1 |
| 193 | A1-M-B44 | 1.33 | 336.1 |
| 194 | A9-M-B44 | 1.36 | 336.1 |
| 195 | A3-M-B44 | 1.21 | 312.1 |
| 196 | A1-M-B45 | 1.4 | 352.1 |
| 197 | A8-M-B45 | 1.39 | 398.1 |
| 198 | A3-M-B45 | 1.29 | 328.1 |
| 199 | A1-M-B46 | 1.39 | 310.2 |
| 200 | A8-M-B46 | 1.38 | 356.2 |
| 201 | A3-M-B46 | 1.27 | 286.1 |
| 202 | A1-M-B47 | 1.28 | 282.2 |
| 203 | A2-M-B47 | 1.28 | 282.2 |
| 204 | A8-M-B47 | 1.25 | 328.2 |
| 205 | A3-M-B47 | 1.12 | 258.1 |
| 206 | A1-M-B48 | 1.27 | 284.2 |
| 207 | A9-M-B48 | 1.3 | 284.2 |
| 208 | A2-M-B48 | 1.3 | 284.2 |
| 209 | A6-M-B48 | 1.19 | 276.1 |
| 210 | A8-M-B48 | 1.26 | 330.2 |
| 211 | A7-M-B48 | 1.22 | 300.2 |
| 212 | A3-M-B48 | 1.14 | 260.1 |
| 213 | A1-M-B49 | 1.39 | 362.2 |
| 214 | A2-M-B49 | 1.42 | 362.2 |
| 215 | A8-M-B49 | 1.38 | 408.2 |
| 216 | A3-M-B49 | 1.28 | 338.1 |
| 217 | A1-M-B50 | 1.13 | 285.2 |
| 218 | A9-M-B50 | 1.34 | 285.2 |
| 219 | A2-M-B50 | 1.18 | 285.2 |
| 220 | A6-M-B50 | 1.05 | 277.1 |
| 221 | A7-M-B50 | 1.1 | 301.2 |
| 222 | A3-M-B50 | 1 | 261.1 |
| 223 | A1-M-B51 | 1.33 | 333.2 |
| 224 | A2-M-B51 | 1.37 | 333.2 |
| 225 | A1-M-B52 | 1.41 | 397.1 |
| 226 | A9-M-B52 | 1.44 | 397.1 |
| 227 | A2-M-B52 | 1.45 | 397.1 |
| 228 | A6-M-B52 | 1.35 | 389.0 |
| 229 | A8-M-B52 | 1.42 | 443.1 |
| 230 | A1-M-B53 | 1.31 | 349.2 |
| 231 | A9-M-B53 | 1.31 | 349.2 |
| 232 | A2-M-B53 | 1.31 | 349.2 |
| 233 | A6-M-B53 | 1.21 | 341.1 |
| 234 | A10-M-B54 | 1.26 | 392.1 |
| 235 | A11-M-B55 | 1.41 | 374.1 |
| 236 | A1-M-B56 | 1.05 | 271.1 |
| 237 | A9-M-B56 | 1.09 | 271.1 |
| 238 | A2-M-B56 | 1.09 | 271.1 |
| 239 | A6-M-B56 | 0.97 | 263.1 |
| 240 | A8-M-B56 | 1.08 | 317.2 |
| 241 | A1-M-B57 | 1.4 | 325.2 |
| 242 | A9-M-B57 | 1.33 | 325.2 |
| 243 | A2-M-B57 | 1.33 | 325.2 |
| 244 | A6-M-B57 | 1.23 | 317.1 |
| 245 | A8-M-B57 | 1.31 | 371.2 |
| 246 | A1-M-B58 | 1.28 | 355.1 |
| 247 | A2-M-B58 | 1.31 | 355.1 |
| 248 | A1-M-B59 | 1.28 | 337.1 |
| 249 | A9-M-B59 | 1.32 | 337.1 |
| 250 | A2-M-B59 | 1.32 | 337.1 |
| 251 | A6-M-B59 | 1.22 | 329.1 |
| 252 | A1-M-B60 | 1.39 | 353.1 |
| 253 | A2-M-B60 | 1.43 | 353.1 |
| 254 | A6-M-B60 | 1.33 | 345.0 |
| 255 | A1-M-B61 | 1.24 | 349.2 |
| 256 | A9-M-B61 | 1.27 | 349.2 |
| 257 | A2-M-B61 | 1.27 | 349.2 |
| 258 | A6-M-B61 | 1.17 | 341.1 |
| 259 | A8-M-B61 | 1.25 | 395.2 |
| 260 | A1-M-B62 | 1.47 | 361.2 |
| 261 | A9-M-B62 | 1.5 | 361.2 |
| 262 | A2-M-B62 | 1.5 | 361.2 |
| 263 | A6-M-B62 | 1.41 | 353.1 |
| 264 | A8-M-B62 | 1.48 | 407.2 |
| 265 | A1-M-B63 | 1.27 | 347.2 |
| 266 | A9-M-B63 | 1.3 | 347.2 |
| 267 | A2-M-B63 | 1.3 | 347.2 |
| 268 | A6-M-B63 | 1.35 | 339.1 |
| 269 | A8-M-B63 | 1.29 | 393.2 |
| 270 | A1-M-B64 | 1.36 | 353.1 |
| 271 | A12-M-B64 | 1.34 | 369.1 |
| 272 | A1-M-B65 | 1.38 | 353.1 |
| 273 | A12-M-B65 | 1.38 | 369.1 |
| 274 | A8-M-B65 | 1.4 | 399.1 |
| 275 | A1-M-B66 | 1.32 | 337.1 |
| 276 | A12-M-B66 | 1.32 | 353.1 |
| 277 | A2-M-B66 | 1.49 | 337.1 |
| 278 | A6-M-B66 | 1.26 | 329.1 |
| 279 | A1-M-B67 | 1.3 | 313.2 |
| 280 | A12-M-B67 | 1.29 | 329.2 |
| 281 | A2-M-B67 | 1.34 | 313.2 |
| 282 | A6-M-B67 | 1.23 | 305.1 |
| 283 | A8-M-B67 | 1.32 | 359.2 |
| 284 | A1-M-B68 | 1.23 | 361.2 |
| 285 | A12-M-B68 | 1.22 | 377.2 |
| 286 | A2-M-B68 | 1.27 | 361.2 |
| 287 | A1-M-B69 | 1.33 | 347.2 |
| 288 | A12-M-B69 | 1.32 | 363.2 |
| 289 | A2-M-B69 | 1.36 | 347.2 |
| 290 | A8-M-B69 | 1.34 | 393.2 |
| 291 | A1-M-B70 | 1.33 | 351.2 |
| 292 | A12-M-B70 | 1.31 | 367.1 |
| 293 | A1-M-B71 | 1.57 | 347.2 |
| 294 | A12-M-B71 | 1.38 | 363.2 |
| 295 | A2-M-B71 | 1.41 | 347.2 |
| 296 | A6-M-B71 | 1.31 | 339.1 |
| 297 | A8-M-B71 | 1.39 | 393.2 |
| 298 | A1-M-B72 | 1.35 | 355.1 |
| 299 | A12-M-B72 | 1.35 | 371.1 |
| 300 | A1-M-B73 | 1.22 | 361.2 |
| 301 | A12-M-B73 | 1.21 | 377.2 |
| 302 | A2-M-B73 | 1.26 | 361.2 |
| 303 | A1-M-B74 | 1.52 | 392.1 |
| 304 | A12-M-B74 | 1.49 | 408.1 |
| 305 | A2-M-B74 | 1.54 | 392.1 |
| 306 | A1-M-B75 | 1.37 | 359.1 |
| 307 | A12-M-B75 | 1.35 | 375.1 |
| 308 | A2-M-B75 | 1.4 | 359.1 |
| 309 | A1-M-B76 | 1.36 | 400.1 |
| 310 | A12-M-B76 | 1.35 | 416.1 |
| 311 | A2-M-B76 | 1.4 | 400.1 |
| 312 | A1-M-B77 | 1.49 | 374.1 |
| 313 | A12-M-B77 | 1.46 | 390.1 |
| 314 | A2-M-B77 | 1.52 | 374.1 |
| 315 | A1-M-B78 | 1.43 | 374.1 |
| 316 | A12-M-B78 | 1.41 | 390.1 |
| 317 | A2-M-B78 | 1.46 | 374.1 |
| 318 | A1-M-B79 | 1.28 | 306.1 |
| 319 | A12-M-B79 | 1.27 | 322.1 |
| 320 | A2-M-B79 | 1.32 | 306.1 |
| 321 | A1-M-B80 | 1.51 | 380.0 |
| 322 | A12-M-B80 | 1.49 | 396.0 |
| 323 | A2-M-B80 | 1.55 | 380.0 |
| 324 | A1-M-B81 | 1.18 | 382.2 |
| 325 | A1-M-B82 | 1.37 | 365.1 |
| 326 | A1-M-B83 | 1.23 | 311.2 |
| 327 | A2-M-B83 | 1.27 | 311.2 |
| 328 | A2-M-B84 | 1.19 | 278.1 |
| 329 | A12-M-B85 | 1.42 | 370.1 |
| 330 | A2-M-B85 | 1.47 | 354.1 |
| 331 | A12-M-B86 | 1.47 | 390.1 |
| 332 | A1-M-B87 | 1.51 | 418.0 |
| 333 | A12-M-B87 | 1.75 | 434.0 |
| 334 | A1-M-B88 | 1.2 | 292.1 |
| 335 | A2-M-B88 | 1.24 | 292.1 |
| 336 | A1-M-B89 | 1.39 | 358.1 |
| 337 | A12-M-B89 | 1.37 | 374.1 |
| 338 | A2-M-B89 | 1.42 | 358.1 |
| 339 | A1-M-B54 | 1.36 | 346.1 |
| 340 | A12-M-B54 | 1.34 | 362.1 |
| 341 | A2-M-B54 | 1.4 | 346.1 |
| 342 | A1-M-B55 | 1.41 | 358.1 |
| 343 | A12-M-B55 | 1.39 | 374.1 |
| 344 | A2-M-B55 | 1.44 | 358.1 |
| 345 | A1-M-B90 | 1.52 | 424.0 |
| 346 | A1-M-B91 | 1.32 | 400.1 |
| 347 | A2-M-B91 | 1.36 | 400.1 |
| 348 | A1-M-B92 | 1.42 | 358.1 |
| 349 | A12-M-B92 | 1.4 | 374.1 |
| 350 | A2-M-B92 | 1.45 | 358.1 |
| 351 | A1-M-B93 | 1.44 | 354.1 |
| 352 | A12-M-B93 | 1.42 | 370.1 |
| 353 | A2-M-B93 | 1.47 | 354.1 |
| 354 | A1-M-B94 | 1.49 | 448.0 |
| 355 | A12-M-B94 | 1.46 | 464.0 |
| 356 | A2-M-B94 | 1.52 | 448.0 |
| 357 | A13-M-B1 | 1.24 | 336.1 |
| 358 | A14-M-B1 | 1.3 | 318.2 |
| 359 | A13-M-B2 | 1.3 | 350.1 |
| 360 | A14-M-B2 | 1.41 | 332.2 |
| 361 | A15-M-B3 | 1.44 | 324.2 |
| 362 | A13-M-B3 | 1.38 | 342.2 |
| 363 | A16-M-B3 | 1.42 | 340.2 |
| 364 | A15-M-B5 | 1.58 | 338.2 |
| 365 | A17-M-B5 | 1.35 | 360.0 |
| 366 | A13-M-B5 | 1.48 | 356.2 |
| 367 | A18-M-B5 | 1.28 | 300.2 |
| 368 | A11-M-B5 | 1.47 | 340.2 |
| 369 | A17-M-B6 | 1.21 | 373.9 |
| 370 | A13-M-B6 | 1.36 | 370.1 |
| 371 | A13-M-B7 | 1.29 | 380.1 |
| 372 | A16-M-B7 | 1.34 | 378.2 |
| 373 | A17-M-B8 | 1.08 | 384.0 |
| 374 | A15-M-B10 | 1.43 | 312.2 |
| 375 | A10-M-B10 | 1.22 | 344.2 |
| 376 | A17-M-B10 | 1.19 | 334.0 |
| 377 | A13-M-B10 | 1.36 | 330.2 |
| 378 | A11-M-B10 | 1.33 | 314.2 |
| 379 | A16-M-B10 | 1.41 | 328.2 |
| 380 | A15-M-B11 | 1.35 | 336.1 |
| 381 | A17-M-B11 | 1.12 | 358.0 |
| 382 | A13-M-B11 | 1.28 | 354.1 |
| 383 | A14-M-B11 | 1.38 | 336.1 |
| 384 | A15-M-B12 | 1.29 | 353.1 |
| 385 | A13-M-B12 | 1.22 | 371.1 |
| 386 | A19-M-B12 | 1.15 | 369.1 |
| 387 | A20-M-B12 | 1.29 | 377.0 |
| 388 | A15-M-B13 | 1.32 | 324.1 |
| 389 | A17-M-B13 | 1.07 | 345.9 |
| 390 | A13-M-B13 | 1.25 | 342.1 |
| 391 | A15-M-B14 | 1.25 | 378.2 |
| 392 | A17-M-B14 | 1.02 | 400.0 |
| 393 | A13-M-B14 | 1.18 | 396.1 |
| 394 | A15-M-B15 | 1.24 | 282.2 |
| 395 | A13-M-B15 | 1.16 | 300.1 |
| 396 | A11-M-B15 | 1.14 | 284.1 |
| 397 | A15-M-B16 | 1.32 | 348.2 |
| 398 | A17-M-B16 | 1.09 | 370.0 |
| 399 | A14-M-B16 | 1.35 | 348.2 |
| 400 | A13-M-B17 | 1.14 | 288.1 |
| 401 | A17-M-B18 | 1.18 | 376.0 |
| 402 | A13-M-B18 | 1.34 | 372.1 |
| 403 | A17-M-B19 | 1.1 | 370.0 |
| 404 | A13-M-B19 | 1.25 | 366.1 |
| 405 | A11-M-B19 | 1.23 | 350.1 |
| 406 | A16-M-B19 | 1.3 | 364.2 |
| 407 | A15-M-B20 | 1.23 | 337.2 |
| 408 | A17-M-B20 | 0.95 | 359.0 |
| 409 | A13-M-B20 | 1.15 | 355.1 |
| 410 | A11-M-B20 | 1.14 | 339.1 |
| 411 | A14-M-B20 | 1.26 | 337.2 |
| 412 | A13-M-B21 | 1.27 | 354.1 |
| 413 | A11-M-B21 | 1.25 | 338.1 |
| 414 | A14-M-B21 | 1.38 | 336.1 |
| 415 | A17-M-B23 | 1.23 | 368.0 |
| 416 | A13-M-B23 | 1.36 | 364.1 |
| 417 | A15-M-B25 | 1.4 | 352.1 |
| 418 | A13-M-B25 | 1.3 | 370.1 |
| 419 | A19-M-B25 | 1.24 | 368.1 |
| 420 | A17-M-B26 | 1.04 | 365.0 |
| 421 | A13-M-B26 | 1.22 | 361.1 |
| 422 | A17-M-B27 | 1.07 | 318.0 |
| 423 | A13-M-B27 | 1.26 | 314.1 |
| 424 | A16-M-B27 | 1.31 | 312.2 |
| 425 | A17-M-B28 | 1.2 | 376.0 |
| 426 | A13-M-B28 | 1.33 | 372.1 |
| 427 | A11-M-B29 | 1.02 | 288.1 |
| 428 | A14-M-B29 | 1.16 | 286.1 |
| 429 | A19-M-B29 | 0.99 | 302.1 |
| 430 | A16-M-B29 | 1.1 | 302.1 |
| 431 | A17-M-B95 | 1.22 | 373.9 |
| 432 | A13-M-B95 | 1.37 | 370.1 |
| 433 | A17-M-B31 | 1.16 | 376.0 |
| 434 | A13-M-B31 | 1.32 | 372.1 |
| 435 | A14-M-B31 | 1.41 | 354.1 |
| 436 | A19-M-B31 | 1.25 | 370.1 |
| 437 | A15-M-B32 | 1.37 | 336.1 |
| 438 | A17-M-B32 | 1.12 | 358.0 |
| 439 | A13-M-B32 | 1.29 | 354.1 |
| 440 | A11-M-B32 | 1.26 | 338.1 |
| 441 | A14-M-B34 | 1.6 | 340.2 |
| 442 | A19-M-B34 | 1.42 | 356.2 |
| 443 | A20-M-B34 | 1.58 | 364.2 |
| 444 | A16-M-B34 | 1.54 | 356.2 |
| 445 | A14-M-B90 | 1.62 | 438.0 |
| 446 | A15-M-B96 | 1.6 | 404.1 |
| 447 | A14-M-B35 | 1.42 | 396.1 |
| 448 | A13-M-B36 | 1.29 | 356.1 |
| 449 | A15-M-B37 | 1.52 | 360.2 |
| 450 | A17-M-B37 | 1.31 | 382.0 |
| 451 | A13-M-B37 | 1.44 | 378.2 |
| 452 | A11-M-B37 | 1.42 | 362.2 |
| 453 | A17-M-B38 | 1.4 | 412.0 |
| 454 | A13-M-B38 | 1.52 | 408.2 |
| 455 | A17-M-B97 | 1.36 | 416.0 |
| 456 | A13-M-B97 | 1.47 | 412.1 |
| 457 | A15-M-B40 | 1.37 | 354.1 |
| 458 | A17-M-B40 | 1.12 | 376.0 |
| 459 | A13-M-B40 | 1.28 | 372.1 |
| 460 | A14-M-B40 | 1.38 | 354.1 |
| 461 | A16-M-B40 | 1.33 | 370.1 |
| 462 | A17-M-B41 | 1.23 | 417.9 |
| 463 | A13-M-B41 | 1.37 | 414.0 |
| 464 | A13-M-B42 | 1.32 | 350.1 |
| 465 | A20-M-B45 | 1.48 | 390.0 |
| 466 | A17-M-B46 | 1.25 | 346.0 |
| 467 | A13-M-B46 | 1.4 | 342.2 |
| 468 | A15-M-B47 | 1.33 | 296.2 |
| 469 | A17-M-B47 | 1.08 | 318.0 |
| 470 | A13-M-B47 | 1.27 | 314.1 |
| 471 | A15-M-B48 | 1.35 | 298.2 |
| 472 | A10-M-B48 | 1.14 | 330.2 |
| 473 | A17-M-B48 | 1.1 | 320.0 |
| 474 | A13-M-B48 | 1.28 | 316.1 |
| 475 | A11-M-B48 | 1.26 | 300.2 |
| 476 | A14-M-B48 | 1.39 | 298.2 |
| 477 | A19-M-B48 | 1.21 | 314.1 |
| 478 | A20-M-B48 | 1.36 | 322.1 |
| 479 | A15-M-B50 | 1.21 | 299.2 |
| 480 | A10-M-B50 | 1.04 | 331.2 |
| 481 | A17-M-B50 | 0.94 | 321.0 |
| 482 | A14-M-B50 | 1.25 | 299.2 |
| 483 | A15-M-B51 | 1.4 | 347.2 |
| 484 | A17-M-B51 | 1.19 | 369.0 |
| 485 | A13-M-B51 | 1.34 | 365.1 |
| 486 | A11-M-B51 | 1.33 | 349.2 |
| 487 | A20-M-B51 | 1.43 | 371.1 |
| 488 | A17-M-B52 | 1.29 | 432.9 |
| 489 | A13-M-B52 | 1.42 | 429.0 |
| 490 | A11-M-B52 | 1.42 | 413.1 |
| 491 | A20-M-B52 | 1.51 | 435.0 |
| 492 | A15-M-B53 | 1.35 | 363.2 |
| 493 | A17-M-B53 | 1.13 | 385.0 |
| 494 | A13-M-B53 | 1.29 | 381.1 |
| 495 | A14-M-B53 | 1.39 | 363.2 |
| 496 | A10-M-B56 | 0.97 | 317.2 |
| 497 | A14-M-B56 | 1.18 | 285.2 |
| 498 | A19-M-B56 | 1.02 | 301.1 |
| 499 | A10-M-B57 | 1.21 | 371.2 |
| 500 | A17-M-B57 | 1.16 | 361.0 |
| 501 | A13-M-B57 | 1.31 | 357.2 |
| 502 | A14-M-B57 | 1.41 | 339.2 |
| 503 | A19-M-B57 | 1.27 | 355.2 |
| 504 | A20-M-B57 | 1.41 | 363.1 |
| 505 | A10-M-B58 | 1.2 | 401.1 |
| 506 | A17-M-B58 | 1.13 | 391.0 |
| 507 | A13-M-B58 | 1.3 | 387.1 |
| 508 | A10-M-B59 | 1.22 | 383.1 |
| 509 | A17-M-B59 | 1.14 | 373.0 |
| 510 | A13-M-B59 | 1.31 | 369.1 |
| 511 | A20-M-B59 | 1.4 | 375.1 |
| 512 | A13-M-B60 | 1.41 | 385.1 |
| 513 | A19-M-B60 | 1.37 | 383.1 |
| 514 | A20-M-B60 | 1.5 | 391.0 |
| 515 | A20-M-B62 | 1.57 | 399.1 |
| 516 | A15-M-B63 | 1.36 | 361.2 |
| 517 | A10-M-B63 | 1.19 | 393.2 |
| 518 | A17-M-B63 | 1.13 | 383.0 |
| 519 | A13-M-B63 | 1.29 | 379.2 |
| 520 | A11-M-B63 | 1.28 | 363.2 |
| 521 | A14-M-B63 | 1.39 | 361.2 |
| 522 | A19-M-B63 | 1.25 | 377.2 |
| 523 | A17-M-B64 | 1.22 | 389.0 |
| 524 | A17-M-B65 | 1.26 | 389.0 |
| 525 | A13-M-B65 | 1.4 | 385.1 |
| 526 | A20-M-B65 | 1.49 | 391.0 |
| 527 | A14-M-B66 | 1.43 | 351.2 |
| 528 | A20-M-B66 | 1.43 | 375.1 |
| 529 | A13-M-B98 | 1.29 | 376.1 |
| 530 | A14-M-B67 | 1.42 | 327.2 |
| 531 | A13-M-B68 | 1.25 | 393.1 |
| 532 | A17-M-B69 | 1.21 | 383.0 |
| 533 | A13-M-B69 | 1.35 | 379.2 |
| 534 | A11-M-B69 | 1.32 | 363.2 |
| 535 | A10-M-B70 | 1.25 | 397.2 |
| 536 | A17-M-B70 | 1.18 | 387.0 |
| 537 | A13-M-B70 | 1.34 | 383.1 |
| 538 | A13-M-B72 | 1.36 | 387.1 |
| 539 | A14-M-B84 | 1.28 | 292.1 |
| 540 | A17-M-B87 | 1.38 | 453.9 |
| 541 | A13-M-B88 | 1.22 | 324.1 |
| 542 | A14-M-B88 | 1.32 | 306.1 |
| 543 | A17-M-B74 | 1.39 | 427.9 |
| 544 | A13-M-B75 | 1.37 | 391.1 |
| 545 | A17-M-B76 | 1.24 | 436.0 |
| 546 | A13-M-B76 | 1.37 | 432.1 |
| 547 | A14-M-B76 | 1.47 | 414.1 |
| 548 | A15-M-B99 | 1.69 | 410.2 |
| 549 | A10-M-B99 | 1.51 | 442.2 |
| 550 | A17-M-B99 | 1.51 | 432.0 |
| 551 | A13-M-B99 | 1.62 | 428.1 |
| 552 | A15-M-B77 | 1.56 | 388.1 |
| 553 | A17-M-B77 | 1.35 | 409.9 |
| 554 | A10-M-B78 | 1.33 | 420.1 |
| 555 | A13-M-B78 | 1.43 | 406.0 |
| 556 | A17-M-B79 | 1.1 | 342.0 |
| 557 | A15-M-B80 | 1.58 | 394.0 |
| 558 | A17-M-B80 | 1.37 | 415.9 |
| 559 | A14-M-B80 | 1.62 | 394.0 |
| 560 | A15-M-B81 | 1.65 | 396.2 |
| 561 | A10-M-B81 | 1.47 | 428.2 |
| 562 | A17-M-B81 | 1.47 | 418.0 |
| 563 | A13-M-B81 | 1.58 | 414.1 |
| 564 | A15-M-B100 | 1.44 | 354.1 |
| 565 | A17-M-B100 | 1.22 | 376.0 |
| 566 | A13-M-B100 | 1.38 | 372.1 |
| 567 | A11-M-B100 | 1.37 | 356.1 |
| 568 | A14-M-B100 | 1.47 | 354.1 |
| 569 | A15-M-B54 | 1.44 | 360.1 |
| 570 | A17-M-B54 | 1.21 | 381.9 |
| 571 | A13-M-B54 | 1.36 | 378.0 |
| 572 | A11-M-B54 | 1.34 | 362.1 |
| 573 | A14-M-B54 | 1.47 | 360.1 |
| 574 | A15-M-B55 | 1.49 | 372.1 |
| 575 | A17-M-B55 | 1.27 | 393.9 |
| 576 | A13-M-B55 | 1.4 | 390.1 |
| 577 | A14-M-B55 | 1.5 | 372.1 |
| 578 | A17-M-B90 | 1.38 | 459.8 |
| 579 | A13-M-B90 | 1.51 | 455.9 |
| 580 | A10-M-B96 | 1.4 | 436.1 |
| 581 | A17-M-B96 | 1.4 | 426.0 |
| 582 | A13-M-B96 | 1.51 | 422.1 |
| 583 | A14-M-B96 | 1.61 | 404.1 |
| 584 | A10-M-B101 | 1.49 | 454.0 |
| 585 | A15-M-B91 | 1.4 | 414.1 |
| 586 | A10-M-B91 | 1.22 | 446.1 |
| 587 | A13-M-B91 | 1.32 | 432.1 |
| 588 | A17-M-B102 | 1.43 | 459.9 |
| 589 | A13-M-B102 | 1.54 | 456.1 |
| 590 | A15-M-B92 | 1.49 | 372.1 |
| 591 | A17-M-B92 | 1.27 | 393.9 |
| 592 | A13-M-B92 | 1.42 | 390.1 |
| 593 | A15-M-B103 | 1.66 | 422.0 |
| 594 | A10-M-B103 | 1.47 | 454.0 |
| 595 | A17-M-B93 | 1.3 | 390.0 |
| 596 | A13-M-B93 | 1.44 | 386.1 |
| 597 | A10-M-B94 | 1.38 | 494.0 |
| 598 | A17-M-B94 | 1.36 | 483.9 |
| 599 | A13-M-B94 | 1.49 | 480.0 |
| 600 | A17-M-B104 | 1.4 | 443.9 |
| 601 | A21-M-B105 | 1.25 | 288.1 |
| 602 | A21-M-B106 | 1.4 | 376.1 |
| 603 | A21-M-B8 | 1.23 | 352.1 |
| 604 | A22-M-B105 | 1.22 | 270.2 |
| 605 | A22-M-B107 | 1.17 | 256.1 |
| 606 | A22-M-B8 | 1.2 | 334.1 |
| 607 | A22-M-B108 | 1.49 | 346.2 |
| 608 | A23-M-B1 | 1.49 | 346.2 |
| 609 | A23-M-B105 | 1.52 | 326.2 |
| 610 | A23-M-B3 | 1.63 | 352.2 |
| 611 | A23-M-B5 | 1.74 | 366.2 |
| 612 | A23-M-B7 | 1.54 | 390.2 |
| 613 | A23-M-B107 | 1.47 | 312.2 |
| 614 | A23-M-B10 | 1.62 | 340.2 |
| 615 | A24-M-B1 | 1.36 | 346.1 |
| 616 | A24-M-B105 | 1.39 | 326.1 |
| 617 | A24-M-B3 | 1.49 | 352.1 |
| 618 | A24-M-B4 | 1.16 | 284.1 |
| 619 | A24-M-B7 | 1.42 | 390.1 |
| 620 | A24-M-B107 | 1.34 | 312.1 |
| 621 | A24-M-B106 | 1.5 | 414.1 |
| 622 | A24-M-B8 | 1.35 | 390.1 |
| 623 | A24-M-B109 | 1.44 | 360.1 |
| 624 | A24-M-B10 | 1.48 | 340.1 |
| 625 | A21-M-B11 | 1.28 | 326.1 |
| 626 | A21-M-B110 | 1.49 | 410.0 |
| 627 | A21-M-B18 | 1.33 | 344.1 |
| 628 | A21-M-B19 | 1.24 | 338.1 |
| 629 | A21-M-B111 | 1.19 | 274.1 |
| 630 | A21-M-B21 | 1.26 | 326.1 |
| 631 | A22-M-B11 | 1.24 | 308.1 |
| 632 | A22-M-B110 | 1.47 | 392.0 |
| 633 | A22-M-B15 | 1.12 | 254.1 |
| 634 | A22-M-B18 | 1.3 | 326.1 |
| 635 | A22-M-B19 | 1.21 | 320.1 |
| 636 | A22-M-B111 | 1.15 | 256.1 |
| 637 | A22-M-B21 | 1.23 | 308.1 |
| 638 | A23-M-B13 | 1.5 | 352.1 |
| 639 | A23-M-B15 | 1.42 | 310.2 |
| 640 | A23-M-B17 | 1.39 | 298.2 |
| 641 | A23-M-B18 | 1.56 | 382.2 |
| 642 | A23-M-B19 | 1.49 | 376.2 |
| 643 | A23-M-B111 | 1.46 | 312.2 |
| 644 | A23-M-B112 | 1.54 | 326.2 |
| 645 | A23-M-B21 | 1.5 | 364.2 |
| 646 | A24-M-B11 | 1.39 | 364.1 |
| 647 | A24-M-B110 | 1.64 | 448.0 |
| 648 | A24-M-B13 | 1.37 | 352.1 |
| 649 | A24-M-B15 | 1.28 | 310.1 |
| 650 | A24-M-B17 | 1.25 | 298.1 |
| 651 | A24-M-B18 | 1.44 | 382.1 |
| 652 | A24-M-B19 | 1.37 | 376.1 |
| 653 | A24-M-B111 | 1.32 | 312.1 |
| 654 | A24-M-B112 | 1.41 | 326.1 |
| 655 | A24-M-B21 | 1.39 | 364.1 |
| 656 | A21-M-B113 | 1.31 | 344.1 |
| 657 | A21-M-B24 | 1.34 | 352.1 |
| 658 | A21-M-B25 | 1.31 | 342.1 |
| 659 | A21-M-B27 | 1.25 | 286.1 |
| 660 | A21-M-B28 | 1.32 | 344.1 |
| 661 | A21-M-B30 | 1.21 | 398.1 |
| 662 | A21-M-B31 | 1.32 | 344.1 |
| 663 | A21-M-B32 | 1.28 | 326.1 |
| 664 | A22-M-B113 | 1.28 | 326.1 |
| 665 | A22-M-B25 | 1.29 | 324.1 |
| 666 | A22-M-B27 | 1.2 | 268.1 |
| 667 | A22-M-B28 | 1.29 | 326.1 |
| 668 | A22-M-B30 | 1.17 | 380.2 |
| 669 | A22-M-B31 | 1.27 | 326.1 |
| 670 | A22-M-B32 | 1.25 | 308.1 |
| 671 | A23-M-B113 | 1.53 | 382.2 |
| 672 | A23-M-B23 | 1.61 | 374.2 |
| 673 | A23-M-B24 | 1.57 | 390.2 |
| 674 | A23-M-B25 | 1.56 | 380.1 |
| 675 | A23-M-B27 | 1.51 | 324.2 |
| 676 | A23-M-B30 | 1.45 | 436.2 |
| 677 | A23-M-B31 | 1.55 | 382.2 |
| 678 | A24-M-B113 | 1.42 | 382.1 |
| 679 | A24-M-B23 | 1.48 | 374.1 |
| 680 | A24-M-B24 | 1.46 | 390.1 |
| 681 | A24-M-B25 | 1.44 | 380.1 |
| 682 | A24-M-B27 | 1.37 | 324.1 |
| 683 | A24-M-B28 | 1.44 | 382.1 |
| 684 | A24-M-B30 | 1.34 | 436.1 |
| 685 | A24-M-B95 | 1.48 | 380.1 |
| 686 | A24-M-B31 | 1.43 | 382.1 |
| 687 | A24-M-B32 | 1.4 | 364.1 |
| 688 | A21-M-B114 | 1.3 | 352.1 |
| 689 | A21-M-B115 | 1.54 | 444.1 |
| 690 | A21-M-B34 | 1.49 | 330.2 |
| 691 | A21-M-B116 | 1.3 | 352.1 |
| 692 | A21-M-B40 | 1.27 | 344.1 |
| 693 | A21-M-B117 | 1.48 | 376.0 |
| 694 | A22-M-B114 | 1.27 | 334.1 |
| 695 | A22-M-B115 | 1.53 | 426.1 |
| 696 | A22-M-B34 | 1.47 | 312.2 |
| 697 | A22-M-B38 | 1.5 | 362.2 |
| 698 | A22-M-B39 | 1.26 | 383.1 |
| 699 | A22-M-B40 | 1.24 | 326.1 |
| 700 | A22-M-B118 | 1.12 | 280.1 |
| 701 | A23-M-B33 | 1.56 | 326.2 |
| 702 | A23-M-B114 | 1.54 | 390.2 |
| 703 | A23-M-B115 | 1.74 | 482.2 |
| 704 | A23-M-B34 | 1.75 | 368.3 |
| 705 | A23-M-B36 | 1.52 | 366.2 |
| 706 | A23-M-B38 | 1.74 | 418.2 |
| 707 | A23-M-B116 | 1.54 | 390.2 |
| 708 | A23-M-B39 | 1.52 | 439.2 |
| 709 | A23-M-B40 | 1.52 | 382.2 |
| 710 | A23-M-B118 | 1.42 | 336.2 |
| 711 | A24-M-B33 | 1.41 | 326.1 |
| 712 | A24-M-B114 | 1.42 | 390.1 |
| 713 | A24-M-B115 | 1.64 | 482.1 |
| 714 | A24-M-B34 | 1.63 | 368.2 |
| 715 | A24-M-B36 | 1.39 | 366.1 |
| 716 | A24-M-B116 | 1.41 | 390.1 |
| 717 | A24-M-B40 | 1.4 | 382.1 |
| 718 | A24-M-B41 | 1.48 | 424.0 |
| 719 | A24-M-B118 | 1.28 | 336.1 |
| 720 | A21-M-B119 | 1.35 | 376.0 |
| 721 | A21-M-B120 | 1.36 | 394.1 |
| 722 | A21-M-B50 | 1.12 | 289.1 |
| 723 | A21-M-B121 | 1.27 | 323.1 |
| 724 | A21-M-B51 | 1.33 | 337.1 |
| 725 | A21-M-B53 | 1.29 | 353.1 |
| 726 | A22-M-B119 | 1.32 | 358.0 |
| 727 | A22-M-B120 | 1.32 | 376.1 |
| 728 | A22-M-B50 | 1.09 | 271.1 |
| 729 | A22-M-B121 | 1.24 | 305.1 |
| 730 | A22-M-B51 | 1.31 | 319.1 |
| 731 | A22-M-B53 | 1.26 | 335.1 |
| 732 | A22-M-B122 | 1.22 | 285.2 |
| 733 | A23-M-B119 | 1.59 | 414.1 |
| 734 | A23-M-B120 | 1.59 | 432.2 |
| 735 | A23-M-B44 | 1.54 | 378.2 |
| 736 | A23-M-B45 | 1.63 | 394.2 |
| 737 | A23-M-B49 | 1.57 | 404.2 |
| 738 | A23-M-B50 | 1.38 | 327.2 |
| 739 | A23-M-B51 | 1.56 | 375.2 |
| 740 | A23-M-B53 | 1.51 | 391.2 |
| 741 | A23-M-B122 | 1.49 | 341.2 |
| 742 | A24-M-B120 | 1.48 | 432.1 |
| 743 | A24-M-B44 | 1.43 | 378.1 |
| 744 | A24-M-B46 | 1.5 | 352.1 |
| 745 | A24-M-B50 | 1.24 | 327.1 |
| 746 | A24-M-B121 | 1.37 | 361.1 |
| 747 | A24-M-B51 | 1.43 | 375.1 |
| 748 | A24-M-B53 | 1.38 | 391.1 |
| 749 | A24-M-B122 | 1.37 | 341.1 |
| 750 | A22-M-B56 | 1.02 | 257.1 |
| 751 | A22-M-B57 | 1.27 | 311.2 |
| 752 | A22-M-B123 | 1.43 | 373.1 |
| 753 | A22-M-B59 | 1.27 | 323.1 |
| 754 | A22-M-B124 | 1.09 | 271.1 |
| 755 | A22-M-B60 | 1.38 | 339.1 |
| 756 | A22-M-B125 | 1.23 | 323.1 |
| 757 | A22-M-B126 | 1.31 | 319.1 |
| 758 | A22-M-B61 | 1.21 | 335.1 |
| 759 | A23-M-B56 | 1.31 | 313.2 |
| 760 | A23-M-B58 | 1.51 | 397.2 |
| 761 | A23-M-B124 | 1.38 | 327.2 |
| 762 | A23-M-B127 | 1.81 | 497.2 |
| 763 | A23-M-B125 | 1.5 | 379.2 |
| 764 | A23-M-B128 | 1.58 | 429.2 |
| 765 | A23-M-B61 | 1.46 | 391.2 |
| 766 | A24-M-B56 | 1.16 | 313.1 |
| 767 | A24-M-B58 | 1.38 | 397.1 |
| 768 | A24-M-B123 | 1.54 | 429.1 |
| 769 | A24-M-B124 | 1.24 | 327.1 |
| 770 | A24-M-B60 | 1.49 | 395.1 |
| 771 | A24-M-B127 | 1.7 | 497.1 |
| 772 | A24-M-B125 | 1.37 | 379.1 |
| 773 | A24-M-B126 | 1.43 | 375.1 |
| 774 | A24-M-B128 | 1.46 | 429.1 |
| 775 | A24-M-B61 | 1.34 | 391.1 |
| 776 | A22-M-B62 | 1.45 | 347.2 |
| 777 | A22-M-B129 | 1.21 | 319.1 |
| 778 | A22-M-B63 | 1.24 | 333.2 |
| 779 | A22-M-B66 | 1.3 | 323.1 |
| 780 | A22-M-B67 | 1.27 | 299.2 |
| 781 | A22-M-B130 | 1.25 | 333.2 |
| 782 | A22-M-B131 | 1.38 | 333.2 |
| 783 | A23-M-B129 | 1.44 | 375.2 |
| 784 | A23-M-B63 | 1.49 | 389.2 |
| 785 | A23-M-B64 | 1.61 | 395.2 |
| 786 | A23-M-B132 | 1.62 | 405.2 |
| 787 | A23-M-B67 | 1.5 | 355.2 |
| 788 | A24-M-B62 | 1.56 | 403.2 |
| 789 | A24-M-B133 | 1.43 | 411.1 |
| 790 | A24-M-B66 | 1.42 | 379.1 |
| 791 | A24-M-B132 | 1.51 | 405.1 |
| 792 | A24-M-B70 | 1.43 | 393.1 |
| 793 | A22-M-B134 | 1.34 | 351.1 |
| 794 | A22-M-B135 | 1.38 | 333.2 |
| 795 | A22-M-B88 | 1.15 | 278.1 |
| 796 | A22-M-B74 | 1.49 | 378.0 |
| 797 | A22-M-B76 | 1.34 | 386.1 |
| 798 | A22-M-B136 | 1.35 | 356.1 |
| 799 | A22-M-B99 | 1.58 | 382.2 |
| 800 | A22-M-B78 | 1.4 | 360.0 |
| 801 | A22-M-B137 | 1.41 | 362.1 |
| 802 | A22-M-B138 | 1.53 | 394.0 |
| 803 | A23-M-B134 | 1.59 | 407.2 |
| 804 | A23-M-B135 | 1.63 | 389.2 |
| 805 | A23-M-B88 | 1.44 | 334.2 |
| 806 | A23-M-B74 | 1.72 | 434.1 |
| 807 | A23-M-B76 | 1.57 | 442.2 |
| 808 | A23-M-B136 | 1.6 | 412.2 |
| 809 | A23-M-B99 | 1.8 | 438.2 |
| 810 | A23-M-B78 | 1.64 | 416.1 |
| 811 | A23-M-B137 | 1.67 | 418.1 |
| 812 | A23-M-B138 | 1.78 | 450.1 |
| 813 | A24-M-B135 | 1.51 | 389.1 |
| 814 | A24-M-B86 | 1.61 | 416.0 |
| 815 | A24-M-B74 | 1.63 | 434.0 |
| 816 | A24-M-B76 | 1.4 | 442.1 |
| 817 | A24-M-B136 | 1.43 | 412.1 |
| 818 | A24-M-B99 | 1.74 | 438.1 |
| 819 | A24-M-B78 | 1.47 | 416.0 |
| 820 | A24-M-B138 | 1.66 | 450.0 |
| 821 | A22-M-B79 | 1.24 | 292.1 |
| 822 | A22-M-B139 | 1.43 | 394.1 |
| 823 | A22-M-B140 | 1.32 | 306.1 |
| 824 | A22-M-B100 | 1.33 | 326.1 |
| 825 | A22-M-B54 | 1.32 | 332.0 |
| 826 | A22-M-B55 | 1.37 | 344.1 |
| 827 | A22-M-B141 | 1.5 | 376.1 |
| 828 | A23-M-B79 | 1.48 | 348.2 |
| 829 | A23-M-B81 | 1.74 | 424.2 |
| 830 | A23-M-B139 | 1.63 | 450.1 |
| 831 | A23-M-B100 | 1.62 | 382.2 |
| 832 | A23-M-B54 | 1.54 | 388.1 |
| 833 | A23-M-B55 | 1.59 | 400.1 |
| 834 | A23-M-B141 | 1.67 | 432.2 |
| 835 | A23-M-B103 | 1.82 | 450.1 |
| 836 | A23-M-B89 | 1.57 | 400.1 |
| 837 | A24-M-B79 | 1.41 | 348.1 |
| 838 | A24-M-B81 | 1.71 | 424.1 |
| 839 | A24-M-B54 | 1.48 | 388.0 |
| 840 | A24-M-B141 | 1.62 | 432.1 |
| 841 | A24-M-B142 | 1.34 | 348.1 |
| 842 | A12-M-B83 | 1.23 | 327.2 |
| 843 | A1-M-B84 | 1.14 | 278.1 |
| 844 | A12-M-B84 | 1.13 | 294.1 |
| 845 | A1-M-B85 | 1.44 | 354.1 |
| 846 | A1-M-B86 | 1.49 | 374.1 |
| 847 | A2-M-B86 | 1.52 | 374.1 |
| 848 | A2-M-B87 | 1.6 | 418.0 |
| 849 | A1-M-B143 | 1.37 | 354.1 |
| 850 | A12-M-B143 | 1.35 | 370.1 |
| 851 | A2-M-B143 | 1.4 | 354.1 |
| 852 | A12-M-B88 | 1.18 | 308.1 |
| 853 | A22-M-B86 | 1.47 | 360.0 |
| 854 | A23-M-B86 | 1.72 | 416.1 |
| 855 | A24-M-B85 | 1.53 | 396.1 |
| 856 | A13-M-B101 | 1.6 | 440.0 |
| 857 | A10-M-B92 | 1.31 | 404.1 |
| 858 | A13-M-B103 | 1.58 | 440.0 |
| 859 | A10-M-B93 | 1.33 | 400.1 |
| 860 | A15-M-B104 | 1.6 | 422.0 |
| 861 | A21-M-B3 | 1.37 | 314.2 |
| 862 | A22-M-B106 | 1.37 | 358.1 |
| 863 | A22-M-B109 | 1.28 | 304.1 |
| 864 | A22-M-B10 | 1.32 | 284.2 |
| 865 | A23-M-B11 | 1.52 | 364.2 |
| 866 | A21-M-B95 | 1.37 | 342.1 |
| 867 | A22-M-B23 | 1.35 | 318.2 |
| 868 | A22-M-B95 | 1.34 | 324.1 |
| 869 | A23-M-B28 | 1.56 | 382.2 |
| 870 | A23-M-B32 | 1.53 | 364.2 |
| 871 | A21-M-B41 | 1.36 | 386.0 |
| 872 | A22-M-B33 | 1.25 | 270.2 |
| 873 | A22-M-B116 | 1.28 | 334.1 |
| 874 | A22-M-B41 | 1.34 | 368.0 |
| 875 | A22-M-B117 | 1.45 | 358.0 |
| 876 | A21-M-B44 | 1.33 | 340.1 |
| 877 | A22-M-B44 | 1.3 | 322.1 |
| 878 | A22-M-B58 | 1.25 | 341.1 |
| 879 | A22-M-B127 | 1.6 | 441.1 |
| 880 | A23-M-B66 | 1.54 | 379.2 |
| 881 | A6-M-B1 | 1.15 | 296.1 |
| 882 | A8-M-B3 | 1.37 | 356.2 |
| 883 | A4-M-B4 | 1.03 | 288.1 |
| 884 | A5-M-B4 | 1.03 | 248.1 |
| 885 | A2-M-B12 | 1.27 | 339.1 |
| 886 | A2-M-B15 | 1.19 | 268.1 |
| 887 | A8-M-B15 | 1.15 | 314.1 |
| 888 | A6-M-B16 | 1.17 | 326.1 |
| 889 | A8-M-B16 | 1.24 | 380.2 |
| 890 | A8-M-B17 | 1.12 | 302.1 |
| 891 | A8-M-B19 | 1.24 | 380.2 |
| 892 | A7-M-B19 | 1.2 | 350.1 |
| 893 | A6-M-B26 | 1.17 | 321.1 |
| 894 | A3-M-B30 | 1.12 | 370.1 |
| 895 | A9-M-B32 | 1.3 | 322.1 |
| 896 | A8-M-B32 | 1.27 | 368.1 |
| 897 | A8-M-B35 | 1.3 | 428.1 |
| 898 | A3-M-B38 | 1.41 | 352.2 |
| 899 | A2-M-B41 | 1.39 | 382.0 |
| 900 | A6-M-B41 | 1.29 | 374.0 |
| 901 | A1-M-B144 | 1.29 | 364.2 |
| 902 | A9-M-B144 | 1.31 | 364.2 |
| 903 | A6-M-B144 | 1.22 | 356.1 |
| 904 | A7-M-B144 | 1.24 | 380.2 |
| 905 | A6-M-B42 | 1.24 | 310.1 |
| 906 | A3-M-B43 | 1.08 | 278.1 |
| 907 | A9-M-B49 | 1.45 | 362.2 |
| 908 | A6-M-B51 | 1.27 | 325.1 |
| 909 | A8-M-B51 | 1.34 | 379.2 |
| 910 | A11-M-B96 | 1.5 | 406.1 |
| 911 | A17-M-B101 | 1.47 | 443.9 |
| 912 | A1-M-B98 | 1.27 | 344.1 |
| 913 | A2-M-B98 | 1.3 | 344.1 |
| 914 | A6-M-B98 | 1.2 | 336.1 |
| 915 | A6-M-B68 | 1.15 | 353.1 |
| 916 | A8-M-B70 | 1.33 | 397.2 |
| 917 | A12-M-B82 | 1.35 | 381.1 |
| 918 | A15-M-B1 | 1.33 | 318.2 |
| 919 | A17-M-B2 | 1.15 | 354.0 |
| 920 | A15-M-B8 | 1.3 | 362.1 |
| 921 | A13-M-B8 | 1.24 | 380.1 |
| 922 | A14-M-B8 | 1.34 | 362.1 |
| 923 | A17-M-B9 | 1.15 | 370.0 |
| 924 | A19-M-B14 | 1.13 | 394.1 |
| 925 | A13-M-B16 | 1.25 | 366.1 |
| 926 | A19-M-B16 | 1.2 | 364.1 |
| 927 | A20-M-B18 | 1.41 | 378.1 |
| 928 | A14-M-B19 | 1.36 | 348.2 |
| 929 | A20-M-B20 | 1.24 | 361.1 |
| 930 | A16-M-B20 | 1.21 | 353.2 |
| 931 | A17-M-B24 | 1.2 | 384.0 |
| 932 | A14-M-B24 | 1.44 | 362.2 |
| 933 | A10-M-B37 | 1.32 | 392.2 |
| 934 | A19-M-B40 | 1.22 | 370.1 |
| 935 | A14-M-B42 | 1.42 | 332.2 |
| 936 | A13-M-B43 | 1.22 | 334.1 |
| 937 | A20-M-B44 | 1.41 | 374.1 |
| 938 | A11-M-B81 | 1.57 | 398.1 |
| 939 | A17-M-B49 | 1.27 | 398.0 |
| 940 | A13-M-B50 | 1.14 | 317.1 |
| 941 | A14-M-B52 | 1.52 | 411.1 |
| 942 | A10-M-B100 | 1.27 | 386.1 |
| 943 | A19-M-B59 | 1.27 | 367.1 |
| 944 | A17-M-B61 | 1.08 | 385.0 |
| 945 | A17-M-B62 | 1.37 | 397.0 |
| 946 | A13-M-B62 | 1.49 | 393.2 |
| 947 | A14-M-B65 | 1.49 | 367.1 |
| 948 | A13-M-B67 | 1.31 | 345.2 |
| 949 | A14-M-B69 | 1.44 | 361.2 |
| 950 | A17-M-B82 | 1.23 | 401.0 |
| 951 | A13-M-B87 | 1.51 | 450.0 |
| 952 | A13-M-B143 | 1.37 | 386.1 |
| 953 | A19-M-B143 | 1.32 | 384.1 |
| 954 | A11-M-B88 | 1.19 | 308.1 |
| 955 | A15-M-B74 | 1.59 | 406.1 |
| 956 | A13-M-B74 | 1.51 | 424.0 |
| 957 | A14-M-B99 | 1.72 | 410.2 |
| 958 | A15-M-B79 | 1.37 | 320.1 |
| 959 | A13-M-B79 | 1.29 | 338.1 |
| 960 | A14-M-B79 | 1.4 | 320.1 |
| 961 | A9-M-B1 | 1.26 | 304.1 |
| 962 | A8-M-B1 | 1.23 | 350.1 |
| 963 | A9-M-B2 | 1.34 | 318.2 |
| 964 | A8-M-B2 | 1.29 | 364.2 |
| 965 | A7-M-B2 | 1.25 | 334.1 |
| 966 | A9-M-B3 | 1.41 | 310.2 |
| 967 | A23-M-B85 | 1.68 | 396.2 |
| 968 | A6-M-B4 | 0.94 | 234.1 |
| 969 | A6-M-B7 | 1.22 | 340.1 |
| 970 | A7-M-B7 | 1.24 | 364.2 |
| 971 | A6-M-B15 | 1.07 | 260.1 |
| 972 | A7-M-B15 | 1.1 | 284.1 |
| 973 | A7-M-B22 | 1.26 | 312.2 |
| 974 | A2-M-B24 | 1.39 | 348.2 |
| 975 | A6-M-B24 | 1.29 | 340.1 |
| 976 | A8-M-B24 | 1.37 | 394.2 |
| 977 | A9-M-B27 | 1.31 | 282.2 |
| 978 | A6-M-B27 | 1.19 | 274.1 |
| 979 | A8-M-B27 | 1.27 | 328.2 |
| 980 | A2-M-B29 | 1.09 | 272.1 |
| 981 | A7-M-B29 | 1.01 | 288.1 |
| 982 | A8-M-B30 | 1.24 | 440.2 |
| 983 | A9-M-B33 | 1.33 | 284.2 |
| 984 | A7-M-B33 | 1.25 | 300.2 |
| 985 | A24-M-B88 | 1.31 | 334.1 |
| 986 | A2-M-B144 | 1.31 | 364.2 |
| 987 | A8-M-B144 | 1.28 | 410.2 |
| 988 | A3-M-B144 | 1.17 | 340.1 |
| 989 | A8-M-B44 | 1.34 | 382.1 |
| 990 | A7-M-B46 | 1.37 | 326.2 |
| 991 | A9-M-B47 | 1.31 | 282.2 |
| 992 | A7-M-B47 | 1.23 | 298.1 |
| 993 | A7-M-B49 | 1.38 | 378.2 |
| 994 | A8-M-B50 | 1.18 | 331.2 |
| 995 | A9-M-B51 | 1.36 | 333.2 |
| 996 | A8-M-B59 | 1.3 | 383.1 |
| 997 | A8-M-B60 | 1.4 | 399.1 |
| 998 | A8-M-B64 | 1.37 | 399.1 |
| 999 | A8-M-B66 | 1.34 | 383.1 |
| 1000 | A8-M-B68 | 1.24 | 407.2 |
| 1001 | A6-M-B72 | 1.28 | 347.1 |
| 1002 | A8-M-B72 | 1.37 | 401.1 |
| 1003 | A17-M-B1 | 1.09 | 340.0 |
| 1004 | A15-M-B2 | 1.39 | 332.2 |
| 1005 | A16-M-B14 | 1.23 | 394.2 |
| 1006 | A14-M-B15 | 1.27 | 282.2 |
| 1007 | A11-M-B23 | 1.36 | 348.2 |
| 1008 | A13-M-B24 | 1.34 | 380.1 |
| 1009 | A17-M-B25 | 1.15 | 373.9 |
| 1010 | A17-M-B42 | 1.17 | 354.0 |
| 1011 | A16-M-B43 | 1.27 | 332.1 |
| 1012 | A19-M-B52 | 1.39 | 427.0 |
| 1013 | A13-M-B122 | 1.26 | 331.2 |
| 1014 | A13-M-B61 | 1.25 | 381.1 |
| 1015 | A14-M-B61 | 1.36 | 363.2 |
| 1016 | A19-M-B66 | 1.3 | 367.1 |
| 1017 | A11-M-B98 | 1.27 | 360.1 |
| 1018 | A17-M-B68 | 1.06 | 397.0 |
| 1019 | A14-M-B68 | 1.34 | 375.2 |
| 1020 | A19-M-B87 | 1.45 | 448.0 |
| 1021 | A14-M-B75 | 1.47 | 373.1 |
| 1022 | A11-M-B99 | 1.61 | 412.2 |
| 1023 | A13-M-B77 | 1.48 | 406.0 |
| 1024 | A11-M-B77 | 1.47 | 390.1 |
| 1025 | A14-M-B77 | 1.58 | 388.1 |
| 1026 | A14-M-B78 | 1.53 | 388.1 |
| 1027 | A10-M-B90 | 1.41 | 470.0 |
| 1028 | A14-M-B101 | 1.71 | 422.0 |
| 1029 | A10-M-B102 | 1.44 | 470.1 |
| 1030 | A17-M-B103 | 1.46 | 443.9 |
| 1031 | A10-M-B104 | 1.42 | 454.0 |
| 1032 | A13-M-B104 | 1.52 | 440.0 |
| 1033 | A21-M-B1 | 1.23 | 308.1 |
| 1034 | A21-M-B108 | 1.52 | 364.2 |
| 1035 | A21-M-B109 | 1.31 | 322.1 |
| 1036 | A21-M-B10 | 1.36 | 302.2 |
| 1037 | A22-M-B1 | 1.19 | 290.1 |
| 1038 | A22-M-B3 | 1.34 | 296.2 |
| 1039 | A22-M-B4 | 0.99 | 228.1 |
| 1040 | A22-M-B7 | 1.27 | 334.1 |
| 1041 | A23-M-B8 | 1.47 | 390.2 |
| 1042 | A21-M-B15 | 1.16 | 272.1 |
| 1043 | A23-M-B110 | 1.77 | 448.1 |
| 1044 | A21-M-B36 | 1.27 | 328.1 |
| 1045 | A22-M-B46 | 1.36 | 296.2 |
| 1046 | A23-M-B121 | 1.49 | 361.2 |
| 1047 | A23-M-B126 | 1.54 | 375.2 |
| 1048 | A22-M-B85 | 1.4 | 340.1 |

1. A method for treating diseases caused by and/or associated with an altered protein kinase activity which comprises administering to a mammal in need thereof an effective amount of a pyrrolo-pyrazole or pyrazolo-azepine derivative represented by formula (I): 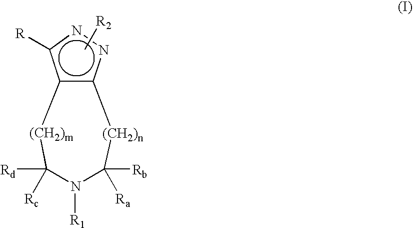
wherein R represents hydrogen or halogen atom, or an optionally substituted group selected from aryl C₂-C₆ alkenyl, (heterocyclyl)C₂-C₆ alkenyl, aryl C₂-C₆ alkynyl, or (heterocyclyl)C₂-C₆ alkynyl group, —R′, —COR′, —COOR′, —CN, —CONR′R″, —OR′, —S(O)_{q}R′, —SO₂NR′R″, —B(OR′″)₂, —SnR″″, wherein R′ and R″, the same or different, independently represent hydrogen atom or an optionally further substituted straight or branched C₁-C₆ alkyl, C₂-C₆ alkenyl, C₂-C₆ alkynyl, saturated or unsaturated C₃-C₆ cycloalkyl, aryl, heterocyclyl, aryl C₁-C₆ alkyl or (heterocyclyl)C₁-C₆ alkyl; R′ represents hydrogen, C₁-C₆ alkyl, or R′″, together with the two oxygen and the boron atoms, forms a saturated or unsaturated C₅-C₈ (hetero)cycloalkyl, optionally benzocondensed or substituted, and R″″ represents C₁-C₆ alkyl;
R₁ represents hydrogen atom or an optionally substituted group selected from —R′, —CH₂R′, —COR′, —COOR′, —CONR′R″, —C(═NH)NHR′, —S(O)_{q}R′, or —SO₂NR′R″, wherein R′ and R″ are as defined above;
R₂represents hydrogen atom, —COR′, —COOR′, —CONR′R″, —S(O)_{q}R′, —SO₂NR′R″, C₁-C₆ alkyl or (heterocyclyl)C₁-C₆ alkyl group, wherein R′ and R″ are as defined above;
Rₐ, R_{b}, R_{c} and R_{d}, being the same or different, independently represent hydrogen atom, an optionally further substituted straight or branched C₁-C₆ alkyl, aryl, heterocyclyl, aryl C₁-C₆ alkyl, (heterocyclyl)C₁-C₆ alkyl or —CH₂OR′ group, wherein R′ is as above defined, or Rₐ and R_{b} and/or R_{c} and R_{d}, taken together with the carbon atom to which they are bonded, form an optionally substituted, saturated or unsaturated, C₃-C₆ cycloalkyl group; q is 0, 1 or 2; m and n, each independently, represents 0, 1 or 2, provided that m+n is 0 or equal to 2; or a pharmaceutically acceptable salt thereof.

2. The method of claim 1 wherein the disease caused by and/or associated with an altered protein kinase activity is selected from the group consisting of cancer, cell proliferative disorders, Alzheimer's disease, viral infections, auto-immune diseases and neurodegenerative disorders.

3. The method of claim 2 wherein the cancer is selected from carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratocanthoma, thyroid follicular cancer and Kaposi's sarcoma.

4. The method of claim 2 wherein the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis glomerulonephritis and post-surgical stenosis and restenosis.

5. The method of claim 1 which provides tumor angiogenesis and metastasis inhibition.

6. The method of claim 1 further comprising subjecting the mammal in need thereof to a radiation therapy or chemotherapy regimen in combination with at least one cytostatic or cytotoxic agent.

7. The method of claim 1 wherein the mammal in need thereof is a human.

8. The method of claim 1 wherein in the compound of formula (I) R is H, I, Br, Cl, F, aryl, C₂-C₆ alkenyl, C₂-C₆ alkynyl, —B(OR′″)₂, —COR′, —CONR′R″, —CN, SO₂R′, OR′, SR′, and R₁ is H, C₁-C₆ alkyl, aryl, —COR′, —CONR′R″, —COOR′, —SO₂R′, or —SO₂NR′R″, and R₂ is H, —COOR′, —COR′, —CONR′R″, C₁-C₆ alkyl, —SO₂R′, or —SO₂NR′R″, (heterocyclyl)C₁-C₆ alkyl group, wherein R′ and R″, the same or different, are selected from hydrogen or optionally substituted straight or branched C₁-C₆ alkyl, aryl or aryl C₁-C₆ alkyl groups;
Rₐ, R_{b}, R_{c} and R_{d}, the same or different, are selected from hydrogen or straight or branched C₁-C₃ alkyl or, taken together with the carbon atom to which they are bonded form a C₃-C₆ cycloalkyl group.

9. The method of claim 1 wherein, in the compound of formula (I), R is selected from aryl, —COR′, —CONR′R″, wherein R′ and R″, the same or different, are selected from hydrogen or optionally substituted straight or branched C₁-C₆ alkyl, aryl or aryl C₁-C₆ alkyl groups.

10. The method of claim 1 wherein, in the compound of formula (I), R₁ is selected from H, C₁-C₆ alkyl, aryl, —COR′, —CONR′R″, COOR′, —SO₂R′ or —SO₂NR′R″, wherein R′ and R″, the same or different, are selected from hydrogen or optionally substituted straight or branched C₁-C₆ alkyl, aryl or aryl C₁-C₆ alkyl groups.

11. The method of claim 1 wherein, in the compound of formula (I), R₂ is H, —COOR′, —CONR′R″, C₁-C₆ alkyl, wherein R′ and R″, the same or different, are selected from hydrogen or optionally substituted straight or branched C₁-C₆ alkyl, aryl or aryl C₁-C₆ alkyl groups.

12. A method for inhibiting protein kinase activity which comprises contacting the said kinase with an effective amount of a compound of formula (I) as defined in claim 1.

13. A pyrrolo-pyrazole or pyrazolo-azepine derivative represented by formula (I): 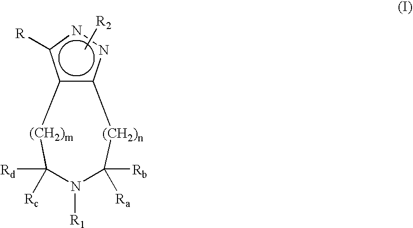
wherein R represents hydrogen or halogen atom, or an optionally substituted group selected from aryl C₂-C₆ alkenyl, (heterocyclyl)C₂-C₆ alkenyl, aryl C₂-C₆ alkynyl, or (heterocyclyl)C₂-C₆ alkynyl group, —R′, —COR′, —COOR′, —CN, —CONR′R″, —OR′, —S(O)_{q}R′, —SO₂NR′R″, —B(OR′″)₂, —SnR″″, wherein R′ and R″, the same or different, independently represent hydrogen atom or an optionally further substituted straight or branched C₁-C₆ alkyl, C₂-C₆ alkenyl, C₂-C₆ alkynyl, saturated or unsaturated C₃-C₆ cycloalkyl, aryl, heterocyclyl, aryl C₁-C₆ alkyl or (heterocyclyl)C₁-C₆ alkyl; R′″ represents hydrogen, C₁-C₆ alkyl, or R′″, together with the two oxygen and the boron atoms, forms a saturated or unsaturated C₅-C₈ (hetero)cycloalkyl, optionally benzocondensed or substituted, and R″″ represents C₁-C₆ alkyl;
R₁ represents hydrogen atom or an optionally substituted group selected from —R′, —CH₂R′, —COR′, —COOR′, —CONR′R″, C(═NH)NHR′, —S(O)_{q}R′, or —SO₂NR′R″, wherein R′ and R″ are as defined above;
R₂ represents hydrogen atom, —COR′, —COOR′, —CONR′R″, —S(O)_{q}R′, —SO₂NR′R″, C₁-C₆ alkyl or (heterocyclyl)C₁-C₆ alkyl group, wherein R′ and R″ are as defined above;
Rₐ, R_{b}, R_{c} and R_{d}, being the same or different, independently represent hydrogen atom, an optionally further substituted straight or branched C₁-C₆ alkyl, aryl, heterocyclyl, aryl C₁-C₆ alkyl, (heterocyclyl)C₁-C₆ alkyl or —CH₂OR′ group, wherein R′ is as above defined, or Rₐ R_{b} and/or R_{c} and R_{d}, taken together with the carbon atom to which they are bonded, form an optionally substituted, saturated or unsaturated, C₃-C₆ cycloalkyl group; q is 0, 1 or 2; m and n, each independently, represents 0, 1 or 2, provided that m +n is 0 or equal to 2 and with the following further provisos:
when m and n are both 1, R is hydrogen atom or hydroxy group and Rₐ, R_{b}, R_{c} and R_{d} are all hydrogen atoms, then R₁ is not hydrogen atom, acetyl, benzyl or ethoxycarbonyl group;
when m is 2 and n is 0, R, Rₐ, R_{b}, R_{c} and R_{d} are all hydrogen atoms, then R₁ is not hydrogen atom or ethoxycarbonyl group;
when m and n are both 0, R, Rₐ, R_{b}, R_{c} and R_{d} are all hydrogen atoms, then R₁ is not hydrogen atom, phenyl-oxazoldinone, quinoline, pyridobenzoxazine or naphtyridine group;
when m and n are both 0, R is propyl, Rₐ, R_{b}, R_{c} and R_{d} are all hydrogen atoms, then R₁ is not phenyl-oxazoldinone group and
when m and n are both 0, R is hydroxy, methyl or ethyl group and Rₐ, R_{b}, R_{c} and R_{d} are all hydrogen atoms, then R₁ is not a methoxycarbonyl group;
or a pharmaceutically acceptable salt thereof

14. A compound of formula (I) according to claim 13 wherein R is H, I, Br, Cl, F, aryl, C₂-C₆ alkenyl, C₂-C₆ alkynyl, —B(OR′″)₂, —COR′, —CONR′R″, —CN, SO₂R′, OR′, SR′, and R₁ is H, C₁-C₆ alkyl, aryl, —COR′, —CONR′R″, —COOR′, —SO₂R′, or —SO₂NR′R″, and R₂ is H, —COOR′, —COR′, —CONR′R″, C₁-C₆ alkyl, —SO₂R′, or —SO₂NR′R″, (heterocyclyl) C₁-C₆ alkyl group, wherein R′ and R″, the same or different, are selected from hydrogen or optionally substituted straight or branched C₁-C₆ alkyl, aryl or aryl C₁-C₆ alkyl groups;
Rₐ, R_{b}, R_{c} and R_{d}, the same or different, are selected from hydrogen or straight or branched C₁-C₃ alkyl or, taken together with the carbon atom to which they are bonded form a C₃-C₆ cycloalkyl group.

15. A compound of formula (I) according to claim 13 wherein R is selected from aryl, —COR′, —CONR′R″, wherein R′ and R″, the same or different, are selected from hydrogen or optionally substituted straight or branched C₁-C₆ alkyl, aryl or aryl C₁-C₆ alkyl groups.

16. A compound of formula (I) according to claim 13 wherein R₁ is selected from H, C₁-C₆ alkyl, aryl, —COR′, —CONR′R″, COOR′, —SO₂R′ or —SO₂NR′R″, wherein R′ and R″, the same or different, are selected from hydrogen or optionally substituted straight or branched C₁-C₆ alkyl, aryl or aryl C₁-C₆ alkyl groups.

17. A compound of formula (I) according to claim 13 wherein R₂ is H, —COOR′, —CONR′R″, C₁-C₆ alkyl, wherein R′ and R″, the same or different, are selected from hydrogen or optionally substituted straight or branched C₁-C₆ alkyl, aryl or aryl C₁-C₆ alkyl groups.

18. A process for preparing the compounds of formula (I) or the pharmaceutically acceptable salts thereof, as defined in claim 13, which process comprises:
a) submitting a compound of formula (II) 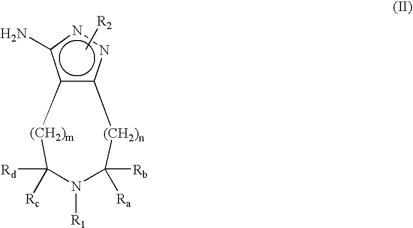
wherein R₁ is as defined in claim 13 but not hydrogen atom, and Rₐ, R_{b}, R_{c}, R_{d}, R₂, m and n are as defined in claim 13, to diazotation and subsequent appropriate quenching, thus obtaining a compound of formula (I) 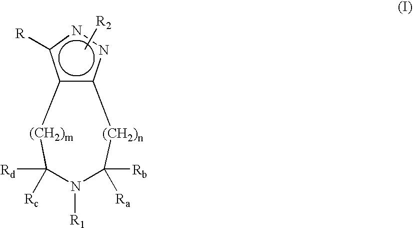
wherein R₁ is as defined above but not hydrogen; Rₐ, R_{b}, R_{c}, R_{d}, R₂, m and n are as defined above, and R is hydrogen, iodine, bromine, chlorine or fluorine atom or a CN group;
b1) converting a thus obtained compound of formula (I) wherein R is I, Br, Cl into another compound of formula (I) wherein R is an optionally substituted aryl, C₂-C₆ alkenyl, C₂-C₆ alkynyl, —SR′, —OR′ or —COR′ wherein R′ is as defined in claim 13;
b2) converting a compound of formula (I) wherein R is hydrogen into another compound of formula (I) wherein R is —B(OR′″)₂, —SnR″″, —COOR′, —COR′, C₁-C₆alkyl or iodine, wherein R′, R′″ and R″″ are as defined in claim 13;
c) converting a compound of formula (I) wherein R is —B(OR′″)₂ or —SnR″″ as above defined into another compound of formula (I) wherein R is an optionally substituted aryl, C₂-C₆ alkenyl, C₂-C₆ alkynyl;
d) optionally converting a compound of formula (I) into another different compound of formula (I), and, if desired, converting a compound of formula (I) into a pharmaceutically acceptable salt thereof or converting a salt into the free compound (I).

19. A process for preparing a compound of formula (I) according to claim 13, which which process comprises:
either b1a) converting a compound of formula (I) into another compound of formula (I) wherein R has the meanings of claim 18 resulting from step b1 and R₁, Rₐ, R_{b}, R_{c}, R_{d}, m and n are as defined in claim 13, analogously to step b1 defined in claim 18 and Pa) reacting the resultant compound of formula (I) wherein R, Rₐ, R_{b}, R_{c}, R_{d}, m and n are as defined above, R₁ is as described above but not hydrogen and R₂ is hydrogen, with a suitable solid support so as to obtain a compound of formula (III) 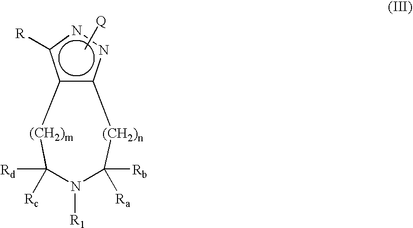
wherein R, Rₐ, R_{b}, R_{c}, R_{d}, m and n are as defined above, R₁ is as defined in claim 13 but not hydrogen, and Q is a solid support, or P) reacting a compound of formula (I) wherein R, Rₐ, R_{b}, R_{c}, R_{d}, m and n are as defined above, R₁ is as defined above but not hydrogen and R₂ is hydrogen, with a suitable solid support so as to obtain a compound of formula (III) as defined above and
B) then, analogously to steps b1, b2, c and d defined in claim 18, optionally converting a thus obtained compound of formula (III) into another compound of formula (III) wherein R has the meanings defined in claim 18 for steps b1 to d and R₁, Rₐ, R_{b}, R_{c}, R_{d}, m and n are as defined above;
C) cleaving a compound of formula (III) so as to eliminate the solid support and to obtain the desired compound of formula (I);
D) optionally converting a compound of formula (I) into another different compound of formula (I),
and, if desired, converting a compound of formula (I) into a pharmaceutically acceptable salt thereof or converting a salt into the free compound (I) as described above.

20. A compound of formula (III) 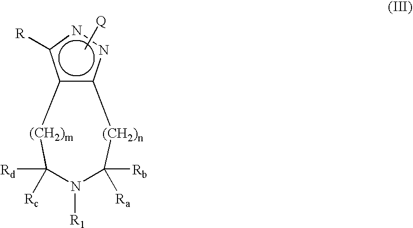
wherein R₁, R, Rₐ, R_{b}, R_{c}, R_{d}, m and n are as defined in claim 13, and Q is a solid support.

21. A compound of formula III according to claim 20 wherein the solid support that Q represents is a residue derived from a resin selected from the group consisting of isocyanate polystyrenic resin, 2-chloro-trityl chloride resin, trityl chloride resin, p-nitrophenyl carbonate Wang resin and the bromo-4-methoxyphenyl)methyl polystyrene.

22. A process for preparing a compound of formula (III) as defined in claim 20, which process comprises:
either b1a) converting a compound of formula (I) into another compound of formula (I) wherein R is as defined in claim 19 resulting from step b1 and R₁, Rₐ, R_{b}, R_{c}, R_{d}, m and n are as defined in claim 13, analogously to step b1 described in claim 18 and Pa) reacting the resultant compound of formula (I) wherein R, Rₐ, R_{b}, R_{c}, R_{d}, m and n are as defined above, R₁ is as defined above but not hydrogen and R₂ is hydrogen, with a suitable solid support so as to obtain a compound of formula (III) 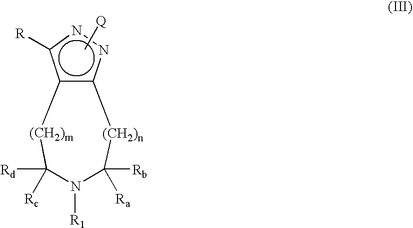
wherein R, Rₐ, R_{b}, R_{c}, R_{d}, m and n are as defined above, R₁ is as defined in claim 13 but not hydrogen, and Q is a solid support, or
A) reacting a compound of formula (I) wherein R, Rₐ, R_{b}, R_{c}, R_{d}, m and n are as defined above, R₁ is as defined above but not hydrogen and R₂ is hydrogen, with a suitable solid support so as to obtain a compound of formula (III) as defined above and
B) then, analogously to steps b1, b2, c and d described in claim 18, optionally converting a thus obtained compound of formula (III) into another compound of formula (III) wherein R has the meanings as defined in claim 18 for steps b1 to d and R₁, Rₐ, R_{b}, R_{c}, R_{d}, m and n are as defined above.

23. A library of two or more compounds of formula (I): 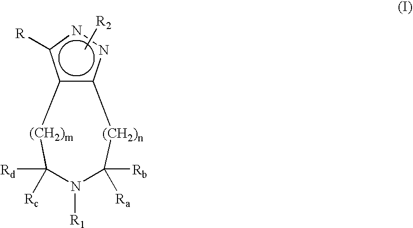
wherein R, R₁, R₂ Rₐ, R_{b}, R_{c}, R_{d} m and n are as defined in claim 13, which can be obtained starting from one or more compound supported onto a solid support of the formula (III) as defined in claim 20.

24. A compound of formula (I) according to claim 13 which is conveniently and unambiguously identified as per the coding system of tables I-III.

25. A pharmaceutical composition comprising a therapeutically effective amount of a compound of formula (I), as defined in claim 13, and at least one pharmaceutically acceptable carrier and/or diluent.

26. A pharmaceutical composition according to claim 24 further comprising one or more chemotherapeutic agents.

27. A product comprising a compound of formula (I) as defined in claim 13 or a pharmaceutical composition thereof as defined in claim 25, and one or more chemotherapeutic agents, as a combined preparation for simultaneous, separate or sequential use in anticancer therapy.

28. A compound of formula (I), as defined in claim 13, for use as a medicament.

29. Use of a compound of formula (I), as defined in claim 1, in the manufacture of a medicament with antitumor activity.
